(19) United States
(12) Patent Application Publication
(10) Pub. No.: US 2003/0009295 A1
(43) Pub. Date: Jan. 9, 2003

(54) System and method for retrieving and using gene expression data from multiple sources

(76) Inventors: Victor Markowitz, Albany, CA (US); Thodoros Topaloglou, Menlo Park, CA (US); I-Min Amy Chen, El Cerrito, CA (US)

Correspondence Address: KILPATRICK STOCKTON LLP, 607 14TH STREET, N.W., SUITE 900, WASHINGTON, DC, 20005, US

(21) Appl. No.: 10/096,645

(22) Filed: Mar. 14, 2002

Related U.S. Application Data
(60) Provisional application No. 60/275,465, filed on Mar. 14, 2001.

(51) Int. Cl. C12Q 1/68; G06F 19/00; G01N 33/48; G01N 33/50

(52) U.S. Cl. 702/20; 435/6

(57) ABSTRACT

A method of analyzing gene expression, gene annotation, and sample information in a relational format supporting efficient exploration and analysis, the method comprising: providing a data warehouse which comprises a gene expression database for storing quantitative gene expression measurements for tissues and cell lines screened using various assays; a clinical database for storing information on bio-samples and donors; and a fragment index for biological properties for DNA fragments; providing a connector which permits loading of more than one source of gene expression, gene annotation, and sample information, receiving a query regarding gene expression of one or more DNA fragments; determining the level of gene expression of the one or more DNA fragments; correlating the level of gene expression with the clinical database and the fragment index; and displaying the results of said correlation is disclosed.

### CROSS-REFERENCE TO RELATED APPLICATIONS

This application claims priority to and incorporates by reference in its entirety, U.S. Patent Provisional Application No. 60/275,465, entitled “A SYSTEM AND METHOD FOR MANAGING GENE EXPRESSION DATA,” filed on Mar. 14, 2001.

### BACKGROUND OF THE INVENTION

1. Field of the Invention

The present invention relates generally to relational databases for storing and retrieving biological information. More particularly the invention relates to systems and methods for retrieving and using gene expression data from multiple sources in systems which provide gene expression, gene annotation, and sample information in a relational format supporting efficient exploration and analysis.

2. Description of the Related Art

DNA microarrays are glass microslides or nylon membranes containing DNA samples (e.g., genomic DNA, cDNA, or oligonucleotides) in an ordered two-dimensional matrix. DNA microarrays can be used to analyze gene expression and genomic clones or to detect single nucleotide polymorphisms (“SNP's”). The DNA used to create a microarray is often from a group of related genes such as those expressed in a particular tissue, during a certain developmental stage, in certain pathways, or after treatment with drugs or other agents. Expression of that group of genes is quantified by measuring the hybridization of fluorescently labeled RNA or DNA to the microarray-linked DNA sequences. By profiling gene expression, transcriptional changes can be monitored through organ and tissue development, microbiological infection, and tumor formation.

Also known as biochips, DNA microarrays can be created by linking monomeric nucleotides on the glass surface to make oligonucleotides. Another methodology, popular for making arrays of polymerase chain reaction (PCR) products and organismal genes, uses robotic instruments to spot thousands of DNA samples onto a surface. This high-throughput approach increases reproducibility and production.

Making the arrays entails transferring 1-2 n1 of DNA sample from 96-1500 well microplates to a 100-200 μm spot on the glass microslide. This is accomplished through single spotting with solid pins or multiple spotting with “split” pins. Output is determined by the number of pins, input microplates, and output microslides. Microarray readers, such as surface fluorometers, are also part of this equation. Since microarrays are used in university research, small and large biopharmaceutical companies, and large-scale clinical trial investigations, there are a variety of instruments and integrated systems to meet these diverse needs.

Affymetrix® of Santa Clara, Calif., provides high-volume production methods that can support the diagnostics or drug development industries. Affymetrix offers GeneChip® technology, which uses glass microarrays manufactured by a proprietary process that combines solid-phase chemistry and photolithography to build probes in situ. The glass wafers are packaged in plastic cartridges in which hybridization is carried out. Several hardware components form the GeneChip suite. The GeneChip Fluidics Station introduces the sample into the probe array cartridge. The Hybridization Oven processes up to 64 cartridges. Agilent Technologies designed its GeneArray® scanner (monochrome; 20 μm resolution) to be used exclusively with Affymetrix microarrays, and the scanner is distributed by Affymetrix for integration into the GeneChip suite. Affymetrix also offers a series of software solutions for data collection, conversion to AADM™ (“Affymetrix Analysis Data Model”) database format, data mining, and a multi-user laboratory information management system (“LIIMS”) system for power-hungry environments.

With today's DNA microarray technology one can easily collect large amounts of data to indicate what genes or SNP's are turned on or turned off during various disease states, following various pharmacological treatments, or following exposure to a variety of toxicological insults. However, while the quantity of data that one can gather with these techniques is very large, it is often out of context. The relevance of genetic data is often determined by its relationship to other pieces of information. For example, knowing that there is an increased expression of a particular gene during the course of a disease is important information. In addition, there is a need to correlate this data with various types of clinical data, for example, a patient's age, sex, weight, stage of clinical development, stage of disease progression, etc.

### BRIEF SUMMARY OF THE INVENTION

It is an object of the present invention to provide a method of analyzing gene expression, gene annotation, and sample information in a relational format supporting efficient exploration and analysis, the method comprising: providing a data warehouse which comprises a gene expression database for storing quantitative gene expression measurements for tissues and cell lines screened using various assays; a clinical database for storing information on bio-samples and donors; and a fragment index for biological properties for DNA fragments; providing a connector which permits loading of more than one source of gene expression, gene annotation, and sample information, receiving a query regarding gene expression of one or more DNA fragments; determining the level of gene expression of the one or more DNA fragments; correlating the level of gene expression with the clinical database and the fragment index; and displaying the results of said correlation.

It is another object of the present invention to provide a computer system comprising a data warehouse which comprises a gene expression database for storing quantitative gene expression measurements for tissues and cell lines screened using various assays; a clinical database for storing information on bio-samples and donors; and a fragment index for biological properties for DNA fragments; a user interface capable of receiving a query regarding gene expression of one or more DNA fragments and displaying the results of a correlation of the level of gene expression with the clinical database and the fragment index; and a connector which permits loading of more than one source of gene expression, gene annotation, and sample information.

### BRIEF DESCRIPTION OF THE DRAWING

FIG. 1 is an illustration of the logical system architecture of the present invention.

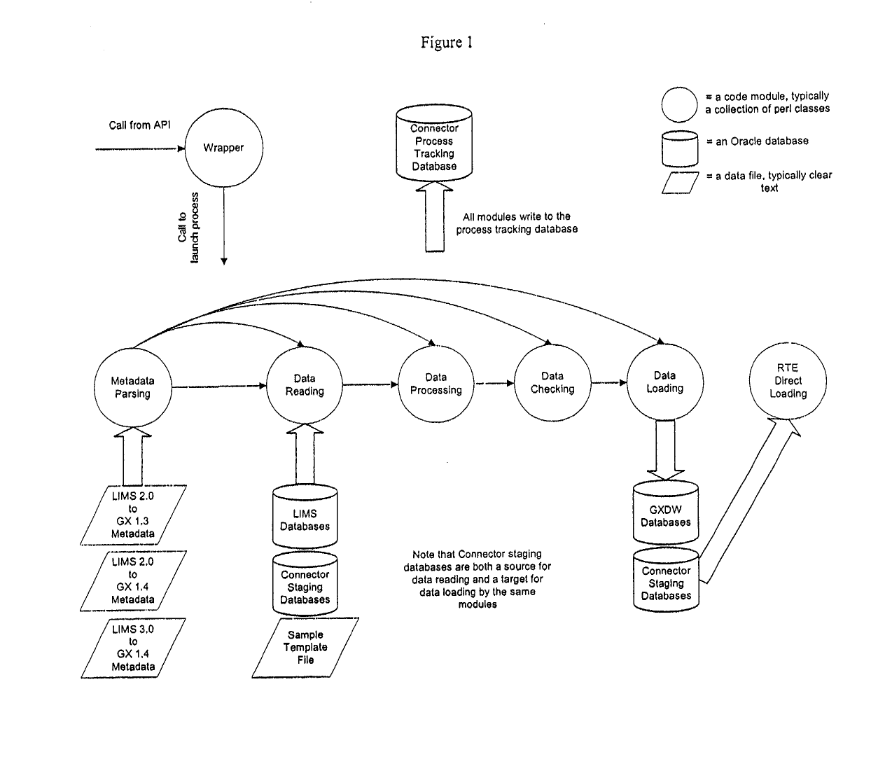

### DETAILED DESCRIPTION OF THE INVENTION

Microarray technologies enable the generation of vast amounts of gene expression data. Effective use of these technologies requires mechanisms to manage and explore large volumes of primary and derived (analyzed) gene expression data. Furthermore, the value of examining the biological meaning of the information is enhanced when set in the context of sample profiles and gene annotation data. The format and interpretation of the data depend strongly on the underlying technology. Hence, exploring gene expression data requires mechanisms for integrating gene expression data across multiple platforms and with sample and gene annotations. The present invention uses data warehousing methodology to manage and explore gene expression and related data.

Generally, the present invention provides a system comprising a data warehouse for storing large amounts of data and having a structure that supports efficient gene expression exploration and analysis. The data warehouse may contain quantitative gene expression information on normal and diseased tissues, experimental animal model and cellular tissues, as well as a variety of treated and untreated conditions. The data warehouse may also contain comprehensive information on samples, clinical profiles, and rich gene annotations.

The connector of the present invention is a tool which permits a user to load of more than one source of gene expression, gene annotation, and sample information, and more particularly, where one of the sources of data is the user's expression data and sample data and a second set of expression and sample data is a standardized set of data, into a data warehouse which comprises a gene expression database for storing quantitative gene expression measurements for tissues and cell lines screened using various assays; a clinical database for storing information on bio-samples and donors; and a fragment index for biological properties for DNA fragments. After the expression and sample data are have been loaded by the connector of the present invention, the user can view, query and analyze his/her own data together with data obtained from another source.

The user's sample data is preferably drawn from a pre-defined sample template in XML format. With the connector of the present invention, a user can also enter or modify the user sample data using an aspect of the present invention, the sample data editor.

There are several preferred features of the connector of the present invention. With regard to gene expression data, these include the ability to register an gene expression data source (LIMS) and extract a list of experiments from this data source; the ability to refresh the experiment list of a registered expression data source; the ability to extract and check a list of “selected” experiments from a registered expression data source, the ability to store the data in a staging database and to record proper status information for the data; the ability to perform gene expression data checking rules; the ability to migrate the expression data from the staging database into the data warehouse; the ability to load expression data into an analysis engine (or Run Time Engine (“RTE”) matrices); the ability to remove the migrated data from the data warehouse and the staging database or corresponding data from the analysis engine (or RTE matrices); and the ability to provide reports for users to check the status of experiments.

With regard to sample data support, preferred features of the connector of the present invention include the ability to provide at least one sample staging database for a user to manage and update his/her sample data; the ability to load and check user sample data from an XML file in a pre-defined sample template data format into the sample staging database; the ability for a user to update his/her sample data using a sample data editor; the ability to load user sample data from the sample staging database into the data warehouse using a “complete refresh” approach; the ability to download sample data in the sample staging database into an output file in XML format (as backup); and the ability to provide reports for users to view user sample data.

With regard to experiment and sample linking for the connector of the present invention, preferred features of the present invention includes the ability for a user to provide association (or links) between experiments and samples; the ability to acquire such linking information from either the XML sample template file or from a connector user interface (“Ul”) expression data migration tool; and the ability to ascertain that the experiment-to-sample links remain fixed for all migrated experiments.

With regard to the user interface for the connector of the present invention, preferred features of the connector include the ability of a user to perform expression and sample data loading; the ability to provide proper status information and messages to a user; the ability to support additional user management functions; the ability to provide a sample data editor; and the ability to provide report viewing functions for a user.

With regard to the application protocol interface (“API”) of the connector, preferred features of the present invention include the ability to provide a set of API calls for the user interface (“UI”) to communicate with the underlying databases.

With regard to the refresh function of the present invention, preferred features of the connector include the ability to preserve user expression and sample data for each data warehouse refresh. In this embodiment, user sample data are loaded into data warehouse using a complete refresh approach. The connector of the present invention preferably tracks the data warehouse sample update schedule (with timestamp and/or flag) to ascertain when a user's complete sample data refresh is required. The gene expression data is preferably partitioned such that the more than one sources of expression data reside in different partitions. The data warehouse refresh will result in the replacement of the expression data partition.

The connector of the present invention allows a user to load and migrate his/her own expression data or sample data into the data warehouse. After the expression and sample data are loaded by the connector, a user is able to view, query and analyze his/her own data together with the standardized data.

Preferably there are two types of connector users (or roles): (1) an administrator and (2) general users. Administrators are the power users who can use the connector to extract experiment data from LIMS and migrate the user data into the data warehouse. Moreover, they can enter user sample data either using the connector sample data editor or through a pre-defined template XML format. Preferably only one administrator can perform data editing and loading. General users preferably cannot enter or load data. They have “read-only” privileges to view expression data or sample data reports provided by the connector of the present invention.

A preferred method for using the connector is through a connector UI or by means of an application launcher. An administrator can prepare user sample data in a pre-defined sample template XML format, and then use the connector to load the sample data. Alternatively, he/she can also use the connector sample data editor (a Java data entry tool) to enter or modify sample data. The user sample data will preferably be staged in at least one connector sample staging database. The user sample data can, thus, be validated by the connector.

Preferably UI operations are translated into API calls to Perl modules to perform the proper system or database operations. Alternatively, an administrator can also invoke these operations using command line calls.

In one preferred embodiment of the present invention, three databases are used by the connector of the present invention. These are:

The expression data staging database which stores all extracted and validated experiments. This database is transient in the sense that experiments or expression data will be truncated after they are loaded into the data warehouse and the analysis engine.

The sample staging database which stores all user sample data. This is also the underlying database for the connector sample data editor. This database is persistent (not transient). However, each sample template data loading from an XML file will preferably wipe out existing sample data in the sample staging database. The sample staging database contents is preferably backed up before each new XML data loading. Therefore, a user can always recover the sample staging database should he/she make a mistake.

The connector process database which stores expression data source (LIMS) information, status and all other information needed by the connector. This is also a persistent (not transient) database. However, deleting LIMS source or unmigrating experiments will cause the corresponding data to be removed from the connector process database.

In other preferred embodiments of the present invention, features include the ability to load user expression data that come in flat file or XML formats; and the ability to load user genomics data using the connector; and the ability for backup and recovery in which RTE matrices can be backed up as files.

Preferably with regard to expression data sources for the connector of the present invention, the connector loads expression data from Affymetrix LIMS Oracle databases. In another preferred embodiment of the present invention, the connector loads expression data from other systems or flat files. If the user's expression data are in other (compatible) types of systems or flat files, then the data is preferably downloaded, processed and uploaded into a LIMS Oracle database.

In a preferred embodiment, the connector of the present invention allows a user to load experiments running on a number of different chip set types, including HG_U95, HG_U95(version 2), Hu42K, HuGeneFL, HG_U133, MG_U74, MG_U74 (version 2), Mu11K, Mu19K, Mu6500, RG_U34, YG_S98, Ye6100, and E. coli.

Preferably with the connector of the present invention, there are 4 major steps in expression data loading:

1. Register and initialize (or refresh) an expression data source: A user first registers an expression data source Oracle database by entering the Oracle database information (TNS name, host name, port number and/or SID) and user logon information (user name and password). All experiments in this Oracle database will be recorded in a master experiment list. When new experiments have been added to a registered expression data source, a user can refresh the master experiment list for this data source.

2. Extract and validate selected experiments into the staging database: A user selects a list of experiments from a registered expression data source. All experiments in the same batch preferably come from the same expression data source. However, a user is preferably allowed to select experiments from different expression data sources in different batches. All expression data sources is preferably registered first. All selected experiments are preferably validated to see whether the data are “complete”. All validated experiments are staged in the expression data staging database for further operations. Proper ID value transformation is performed before data are loaded into the expression data staging database to ascertain that the user expression data and the standardized expression data are using different ID spaces.

3. Link experiments with sample data: Preferably, there are two ways to establish experiment to sample links: specified in the sample XML file, or specified using the connector UI. Each experiment is preferably only associated with one sample. However, multiple experiments can be linked to the same sample.

4. Migrate the data into the data warehouse: Only validated and linked (to sample) experiments can be migrated into the data warehouse. The migrated experiment data will also be loaded to the analysis engine or Run Time Engine (“RTE”).

Preferably there is also an “un-migrate” operation which allows a user to remove migrated experiments from the data warehouse and the analysis engine.

In another preferred embodiment of the present invention, there are 11 expression data checking rules. These rules are applied to expression data to check the data for consistency with a predetermined format, and for completeness. Most of the checking rules apply to individual experiments. Some apply to entire data sources. The rules are listed below with associated actions:

1. Are the “static” tables identical in the staging and target warehouse databases?

Action: re-create expression staging database and re-initialize all expression data sources.

2. Has experiment been published before?

Action: Log (and possibly display in UI) event documenting that experiment being migrated has been published before.

3. Is chip type of experiment consistent with the standardized data?

Action: if chip type is not present in the standardized data, remove experiment from processing, log event documenting that experiment is of an unsupported chip type and failed processing.

4. Does the number of published cells equal the number of expected cells?

Action: if experiment contains an incorrect number of cells, remove from processing, log event documenting that experiment had an incorrect number of cells and failed processing.

5. Is the analysis type of the experiment consistent? Or, was analysis performed on the expected number of biological items?

Action: if analysis was performed on an incorrect number of biological items for the given chip type, remove experiment from processing, log event documenting that experiment had an incorrect number of biological items in analysis and failed processing.

6. Was the experiment's analysis template set to the absolute or relative value?

Action: if analysis template was not set to the absolute or relative value, remove experiment from processing, log event documenting that experiment was not analyzed for absolute results and failed processing.

7. Was the experiment's analysis template set to the relative value?

Action: if analysis template was set to the relative value, warn the user that this is the case and use the absolute expression data for that experiment; ignore the relative expression data.

8. Were the experiments' target (“TGT”) parameters set to standards?

Action: if parameters were not set to standards (i.e., TGT=100), apply translation rule to normalize experiment data to settings, log event documenting that experiment data was normalized, and proceed with processing.

9. Were all biological items analyzed and published?

Action: if all expected biological items were not analyzed and published, remove experiment from processing, log event documenting that analysis/publishing was not complete and experiment failed processing.

10. Do all references to experiments in the analysis tables have corresponding entries in the experiment tables?

Action: if an experiment does not have the proper records in both the analysis and experiment tables, remove experiment from processing, log event documenting that experiment data not complete, and failed processing.

11. Does the experiment have complete analysis records?

Action: if an experiment does not have all of the expected analysis records, remove from processing, log event documenting that experiment's analysis data was not complete, and it failed processing.

Preferably in the connector of the present invention, the selected and validated experiment data are staged in an expression data staging database in the connector. The expression data staging database is preferably an Oracle database with Affymetrix GATC-AADM schema. The expression data staging database is a transient database used internally by the connector. Expression data will be truncated from the expression data staging database after being migrated to the data warehouse and the analysis engine.

Besides the expression data staging database, the connector of the present invention preferably has another internal database called the process staging database. The process staging database keeps track of experiment and batch status. In addition, the process staging database also records information regarding expression data sources, user profiles, experiment-to-sample linking information and sample data loading status. The process staging database is a persistent database. Experiment status information resides in the process staging database even after the experiment data have been migrated to the data warehouse and the analysis engine.

Preferably a user employs a connector expression data migration tool and related UI to link selected and validated experiments to samples. Preferably sample data is loaded or entered into the sample staging database before linking. Experiment-to-sample links can also be defined in the sample template XML file. In a preferred embodiment each experiment can be associated with only one sample. However, many experiments can be associated with the same sample. If a user links a linked experiment to another sample, then the new link will replace the existing link with one exception: migrated experiments (i.e., experiments that have been migrated into the data warehouse cannot be re-linked).

In another preferred embodiment of the present invention, migrated user expression data can be removed from the data warehouse by means of an “un-migrate” function that will remove migrated experiment data from the data warehouse. Gene expression data preferably are never moved from the user's LIMS or expression data sources. A user can preferably always reload those experiments that have been “un-migrated”. All “un-migrated” expression data will be removed (or truncated) from the connector staging databases. The reloading will start from experiment selection and validation (not from warehouse migration).

In a preferred embodiment, an administrator can delete a registered expression data source. Moreover, an expression data source can preferably be removed only when there are no selected and validated or migrated experiments from this data source. A user preferably has to “un-migrate” all experiments from a data source before deleting the data source.

In another preferred embodiment, a user cannot cancel in midstream. However, he/she can always “undo” the operation (e.g., “un-migrate” experiments).

In a preferred embodiment of the connector of the present invention with regard to sample data loading, user sample data are specified in the connector sample template data model. This model contains the following object classes for defining user sample data: sample (defines a user sample object, including sample name, species, organ and disease information), donor (defines donor of a sample, including donor name, age, gender, race and disease information), study (defines a study, including name, description, investigator and comments; a study consists of several study groups), study group (defines a study group, including name, description and comments), and treatment (defines a chemical treatment to a sample, including agent, dosing, regimen, etc.)

Each sample has a single donor. However, many samples can come from the same donor. Each sample can be associated with multiple chemical treatments. A study consists of several study groups. But a study group is limited to a single study. A sample is associated with a single study group and study. User sample data can be entered using XML files that conform to the sample template data model, or using the connector sample data editor (a Java UI data entry tool).

In another embodiment of the present invention, a user can enter sample data that do not fit into the pre-defined sample template data model. A user can enter proprietary data into four object classes (sample, d, study and study group) in the sample template data model. Each piece of proprietary data is a tag-value pair. It can be considered as tag=value or (tag, value) pair. Examples of proprietary data include: “Eye_Color-Black” (eye color is black), “SSN=123456789” (social security number is 123456789). “Tag” shows up as a queryable attribute for the value. It shows up as an independent node called “Proprietary data”.

Preferably the connector supports clinical taxonomies, for example, the SNOMED 3.5 taxonomies for organs (topology) and diseases. Each SNOMED term code (for example, T-01210) is associated with a primary term or name, and may have one or more synonyms. Users can use either primary terms or synonyms in their XML sample data file. The connector will preferably identify the proper SNOMED term code for the terms or synonyms. In the sample data editor, primary terms are preferably provided for a user's selection.

In a preferred embodiment, the user sample data loading is carried as follows.

XML file to sample staging database: This task is done by Perl modules as follows:

1. The XML sample template file is parsed using a Perl XML parser. The parser also performs syntax and reference checking.

2. SQL loader control and data files are generated for loading user sample data into the sample staging database.

3. All sample staging database tables for user sample data are backed up into an XML file and then truncated.

4. Actual data loading is performed using an Oracle SQL loader.

Sample staging database to the sample database in the data warehouse: This task is done by Perl modules as follows:

1. ID mapping between sample staging database objects and the sample database in the data warehouse objects is done for all “new” sample staging database objects.

2. Data are retrieved from the sample staging database based on a metadata control file.

3. SQL loader control and data files are generated for loading sample data into the sample database in the data warehouse.

4. All user sample data in the sample database in the data warehouse are removed using SQL delete statements.

5. User sample data are loaded into the sample database in the data warehouse using Oracle SQL loader.

The “XML file to sample staging database” step and the “sample staging database to the sample database in the data warehouse” step are two individual and separate steps. It is possible to perform 2 or more “XML file to sample staging database” data loading before a final “sample staging database to the sample database in the data warehouse” refresh. It is also preferably possible to perform only “XML file to sample staging database” loading provided that the sample data are entered into the sample staging database using the connector sample data editor.

In a preferred embodiment of the present invention, validation is performed on the sample data. For example, if the user sample data are from an XML template file, then the following rules are checked:

1. All ID values are unique within each class (sample, donor, study, study group and treatment).

2. All ID references refer to existing objects. For example, if a sample X refers to a donor Y, then both X and Y are preferably defined in the same XML file.

3. All attributes take values from “static” controlled vocabulary set must have values from the static set. For example, attribute “type” of the donor class must be from the set {“Human”, “Animal”}.

4. The XML definition preferably conforms to the sample template model. That is, the XML file only contains class and attribute values specified by the sample template model. Each attribute that is specified as “required” will preferably have only non-null values.

5. Experiment-to-sample links are checked to ascertain that all migrated experiments (if any) are still linked to the same sample objects.

If the sample data are entered using the connector sample data editor, then only rule 1 preferably is checked. Rules 2-4 are preferably automatically enforced by the data editor. A user preferably cannot change existing experiment-to-sample links (rule 5) from any UI.

As noted above, in a preferred embodiment, the sample staging database in the connector serves two purposes. It is a place to stage user sample data from an XML file before the data are loaded into sample database in the data warehouse. The sample staging database is also preferably the underlying database for the sample data editor. The sample staging database preferably is an Oracle database designed using OPM. The sample staging database schema preferably consists of 4 major parts:

1. Sample file information: general information (e.g., owner, date) for the XML sample data file.

2. Static controlled vocabulary classes such as donor type, gender, SNOMED disease term and code, SNOMED organ term and code, etc.

3. User sample template data such as sample, donor, study group, study and treatment.

4. ID mapping information between sample staging database Sample and sample database in the data warehouse—for connector internal usage. ID mapping information is kept persistent in the sample staging database to ascertain that the same user sample object will always be assigned with the same ID value in various sample database refreshes.

In an embodiment of the present invention, the user sample data in an XML template format is loaded into the sample staging database. The sample XML data file is parsed by a Perl XML parser. The XML parser also verifies the correctness of the sample data file. If the XML data file passes the syntax checking and validation, then Oracle SQL*Loader control and data files will be generated for bulk loading user sample data into the sample staging database. The existing sample staging database content is preferably backed up in an XML data file. All the tables representing user sample data are truncated. (However, tables for controlled vocabularies and ID mapping information will not be truncated.) The user sample data are preferably then loaded into and staged in the sample staging database waiting to be migrated into the database in the data warehouse.

In an embodiment, user sample data in sample staging database can be downloaded into the sample template XML format. A Perl script is preferably implemented to take a control file to download user sample data in the sample staging database into an output XML file. All user sample data in the sample staging database are preferably preserved in the XML output file. However, the XML output file may not be identical to the original sample template XML file. That is because some attributes with null values can be assigned with default values (e.g., ‘UNKNOWN’) in the sample staging Sample database by the loader.

One can preferably specify experiment to sample data links in the XML sample template file. In the sample template model for the XML file, there is an “Experiment” object class. Experiment class has the following attributes:

id: unique experiment id, which needs to be in format: <LIMS nickname>_<experiment name>; for example: LIMS1_GLGC000123.

name: experiment name; link to EXPERIMENT.NAME in the CHIP_Chip and GX_Chip) databases.

replicate_no: replicate number for the experiment

sample: the user-specified “id” of sample to which the experiment is linked

replacement_flag: whether this experiment replaces a previous experiment

Each Experiment object in the sample XML file actually represents an experiment-to-sample link.

In another embodiment, the sample data entered by the sample data editor can be migrated into the database in the data warehouse. The sample data migration step (moving sample data from the sample staging database to the database in the data warehouse) performs the same regardless sample data in the sample staging database are loaded from XML file or entered using the sample data editor. One major difference between XML sample file and sample data editor is that XML file loading will completely replace the existing sample data in the sample staging database, while the sample data editor performs an “incremental update” to the sample staging database.

In a preferred embodiment, an administrator can update user sample data. There are two preferred ways to update user sample data:

1. Edit a sample XML template file, and then load (i.e., complete re-load) the sample data into the sample staging database.

2. Use the sample data editor to enter new data or modify existing data. The sample data editor will automatically update the sample staging database.

User sample data in the sample staging database is preferably migrated into the sample database in the data warehouse when necessary or by user's request. Experiment-to-sample links for migrated experiments preferably cannot be changed. That is, if a sample is associated with any migrated experiment(s), then the sample cannot be deleted. However, an update is allowed. Also, if the user sample data are loaded from an XML file, and the XML file contains experiment-to-sample links, then experiment-to-sample links must stay the same for migrated experiments. Otherwise, an error message will be reported to the user.

In a preferred aspect of the connector, the connector backs up user sample data by downloading existing user sample data in the sample staging database into an XML sample template file before each new XML sample file loading. Backup sample XML files are named with “timestamp” information. If an administrator loads a new XML data file by mistake (and truncates the existing sample staging database contents), then he/she can always reload user sample data from the most recent XML backup file. The connector will never delete any sample XML backup files. It is preferably the administrator's responsibility to delete old sample XML backup files that are no longer useful to free up disk space. XML sample backup files are generated using the the sample staging database download Perl script. An administrator can perform additional backups.

In another embodiment of the present invention, the database in the data warehouse is refreshed with user sample data. Additionally, upon this refresh further validation is performed. Before sample staging database to database in the data warehouse loading, the connector will preferably check controlled vocabulary tables in the database in the data warehouse to ascertain that they are consistent with corresponding controlled vocabulary tables in the sample staging database. Both databases share the same set of controlled vocabularies. In most cases, the two databases will be consistent.

In a preferred embodiment of the present application, a user starts with a launcher, which is a top-level application that can launch the following independent applications: LIMS (or expression data source) manager, expression data migration, sample data editor, explorer, connector reports, portal, user (login) manager, and sample data manger.

The LIMS (expression data source) manager preferably has 3 major functions: register a new LIMS source and create the master experiment list for this source; refresh the master experiment list for a registered LIMS source; and delete a LIMS source.

The Sample Data Manager preferably provides 3 major functions: upload user sample data from an XML template file to the sample staging database; download existing sample data in the sample staging database into an output XML file (on the server side); and start the database in the data warehouse refresh with user sample data from the sample staging database.

Preferably the connector provides two types of reports to administrators and general users: expression data reports for the user, batch, and individual experiment and sample data reports for the sample, donor, study, and study group.

A user can query and browse expression and sample data using the provided reporting tools.

In a p[referred embodiment of the present invention, the user data source is normalized. The normalized data format is based on qualifier-value pairs submitted by the user or a data entry tool. In the case of user submission, the data values are processed for correcting strings (capitalization, removal of undesirable spaces), mapping to controlled vocabularies, and conversion to standard units.

The normalized data format does not assume any grouping of fields to structured records (objects). In the case of integration projects, there is no requirement of primary and secondary object ids, and null constraint satisfaction. In the case of data entered by a data entry tool, templates preferably supply primary id and null constraint compliance.

If data entry templates are used, the mapping information of data qualifiers to the object model is predefined.

The sample template model is a simplified representation of the sample database that remains unchanged between versions of the sample database. For example, it contains concepts such as sample, donor, study group, study and treatment.

The mapping of the data format to the object model is predefined for standard templates. Properties (attributes) of user sample data can be reflected in the database in the data warehouse preferably only when the data are preserved in the sample template model data.

The sample template data model can be considered as an exemplary OPM schema for user sample data. (That is, it is actually a schema, not a data model.) The key concepts in the object model are: experiment, sample, donor, treatment, study group, and study.

The sample template data model preferably provide an easy way for a user to enter his/her sample data. Most of the attributes in the data model allow null values. User data in the sample template will eventually be inserted into the sample database in the data warehouse. Since the sample database in the data warehouse typically has more restricted constraints on attribute values, many default attribute values or even default pseudo objects will be created when user data are migrated into the data warehouse.

User sample data will be staged in a sample staging database inside the connector. Sample data will be checked for consistencies and controlled vocabularies in certain attributes. Global ID values will be assigned to new objects. Existing sample objects will have the “persistent” ID values based on the use-provided “id” value in sample template and the information in the sample staging database.

User sample data in the sample staging database are then preferably loaded into the sample database in the data warehouse, also using the complete refresh strategy. That is, user sample objects (sample, donor, study, etc.) will use a different ID space than the standardized sample objects. During the complete refresh, all sample objects in the sample database in the data warehouse with ID values in the user ID space will be removed first. Then a complete reloading of user sample data will be performed.

One purpose of the sample staging database is to stage the user sample template data, and to provide a persistent repository of local-global ID value mapping from sample template data to the sample database in the data warehouse. In addition, the sample staging database also stores additional controlled vocabularies (e.g., SNOMED terms and codes) for better sample data mapping.

There are preferably four types of tables in the sample staging database: (1) tables for file header and status metadata, (2) tables to store sample template data, (3) tables to store local-global ID mapping, and (4) tables for complete set of controlled vocabularies (SNOMED and others).

ID Mapping tables store the local-global ID value mapping between user sample data and standardized data. ID mapping information is preferably stored in additional tables (instead of inside the sample template data tables in order to make ID mapping persistent. That is, when a new sample template data file is processed, old data in sample template data tables are truncated. However, data in the ID mapping tables are preferably not truncated. Instead, they will be used as reference tables for persistent local-global ID mapping. An additional “status” attribute is preferably defined for recording data checking result.

In another preferred embodiment of the present invention, user sample data loading process consists of three steps:

1. Uploading (Extraction and Staging): the XML Sample Template data are parsed, and SQL*Load files are generated targeting the sample staging database. Syntax checking is preferably performed. Sample template data tables in the sample staging database are cleaned, and the data into the sample staging database are loaded. Consistency and controlled vocabulary are checked.

2. Transformation: Local (template) and global ID mapping information in the sample staging database are generated.

3. Migration: SQL*Load files are generated targeting the sample database in the data warehouse. The user data in the sample database in the data warehouse (if any) is cleaned, and the data into the sample database in the data warehouse is loaded.

The ID Mapping tables in the sample staging database preferably record persistent local-global ID mapping information. The ID mapping data is re-used for user sample data mapping for existing samples. However, the user sample data file may contain new samples. Therefore, ID Mapping tables need to be updated to append new local-global ID mapping.

The connector architecture preferably is object-oriented so components can be developed and modified individually. Wherever possible, schema-dependent rules and logic are stored outside the code so that schema changes

The connector database and server components preferably run on

1. Hardware: Sun hardware;

2. Operating System: Solaris 5.7;

3. Database: Oracle 8.1.7; AND

4. Other Software: Perl 5.6.

In an embodiment of the present invention, the data warehouse may be modeled as separate sample, gene annotation, and gene expression multi-dimensional data spaces. Basic operations in these data spaces in terms of traditional on-line analytical processing (“OLAP”) dimension reduction and aggregation manipulations may be used for complex gene expression analysis operations. Data warehouse management tools are used for maintaining data consistency, with process specific consistency rules checking the correct execution of data migration and integration processes and with domain specific rules validating sample, expression, and gene annotation data. In accordance with one embodiment of the present invention, an archive may be used to provide a uniform analysis interface for gene expression data from alternate gene expression databases, such as the Genbank public domain database available on the Internet at www.ncbi.nlm.nih.gov/Genbank.

Having briefly described an embodiment of the present invention, basic data warehouse concepts are set forth in order to provide a more thorough understanding of the present invention. The reader should appreciate, however, that the present invention may be practiced without limitation to the specific details presented herein.

Basic Data Warehouse Concepts

A data management infrastructure for gene expression data preferably satisfies two major goals: data acquisition and data analysis. The database technologies needed to address these goals are substantially different. Data acquisition has been a traditional application for operational databases, which are characterized by rapid content substitution as well as the need to support rapid data updates in real time. Generally, operational databases are designed to optimize update performance. In contrast to operational databases, data warehouses are characterized by periodic, rather than real time, content accumulation as well as the need to support rapid exploration of massive amounts of data. Information in data warehouses come from diverse, usually heterogeneous, sources and therefore requires information integration. Generally, data warehouses are designed to optimize query performance for faster data access and for on-line analytical processing.

At the core of a data warehouse is a primary measure attribute associated with a fact object, where the value for the measure attribute is analyzed using the warehouse directly or via an OLAP mechanism. The fact object is modeled in the context of different dimension objects, where each dimension is characterized by one or more category attributes. Category attributes may, in turn, be organized in a specialization hierarchy. A typical example of a data warehouse application involves a product sold in stores on certain dates, where: quantity sold is the measure object, product, store, and date are the associated dimensions, product is characterized by category (e.g., cloth, electronic), store is characterized by location (e.g., city, state), and date is characterized by time (e.g., year, month, day).

OLAP applications view a data warehouse as a multidimensional data space where aggregation functions, such as summarization, can be applied on the measure values. Other OLAP operations include (I) a combination of selection and projection operations, also known as slice and dice operations, which combines a projection on the multidimensional space (slice) with a selection of ranges over the projected dimension (dice); (2) aggregation operations (e.g., summarization) of the measure in a given dimension over one level of the classification hierarchy associated with that dimension, also known as roll-up operations; and (3) disaggregation operations, also known as drill-down operations, which are the reverse of the aggregation operations. For example, a projection operation (slice) can be applied in order to look at the data in a two dimensional space (e.g., location and date); a selection operation (dice) can be used to look at products sold on certain days; and an aggregation operation can be used to summarize quantity sold for a given product category (e.g., electronics).

Unlike traditional data warehouse applications that deal with data representing relatively simple, and precise real-world facts, such as product sales, scientific data in general, and gene expression data in particular, represent complex and often imprecise phenomena. For example, the data may change over time as a reflection of the evolution of the underlying scientific methods used to generate data, and often represent interpretations of experimental results using complex analytical methods.

Accordingly, the complexity of gene expression data entails modeling the data partitioned into three databases: sample, fragment index, and gene expression. Those skilled in the art should appreciate that these databases may require updating, or refreshes, as the underlying scientific methods evolves.

System For Gene Expression Exploration And Analysis

A gene expression data management infrastructure is shown comprising a Data Management System (“DMS”) and a Data Warehouse (“DW”). In accordance with an embodiment of the present invention, DMS comprises operational databases and laboratory information management system (“LIMS”) applications that support data acquisition and management of production data.

In accordance with an embodiment of the present invention, DW comprises summarized and curated gene expression data, integrated with sample and gene annotation data, and provides support for effective data exploration and mining. As previously described, DW may be partitioned into three databases: Sample database, Fragment Index database, and Gene Expression database.

In accordance with an embodiment of the present invention, gene expression data may be generated using the Affymetrix GeneChip platform, marketed by Affymetrix Corporation of Santa Clara, Calif., and may be represented in the Affymetrix Analysis Data Model (“AADM”) relational format extended with specific fields. In the AADM representation, the method dimension for the gene expression data space involves two analysis methods: cell averaging and chip analysis. In one embodiment of the present invention, the results of cell averaging and chip analysis may be stored in two fact tables, the MEASUREMENT_ELEM_RESULT (“MER”) and the ABS_GENE_EXPR_RESULT (“AGER”) tables, respectively. Because of the considerable amount of data contained in DW, the management of both tables may be problematic. For example, one human sample can involve five experiments that result in 1.25 million rows in the MER table and 42,000 rows in the AGER table. Accordingly, in accordance with an embodiment of the present invention, the AGER table may be explored using an OLAP-like multi-dimensional array. Additionally, the MER table may be partitioned and archived. The reader should appreciate that experimental parameters such as protocol version, analysis software build, and analysis method may also be stored in DW.

An archive is provided for storing raw data files generated by microarray experiments. In addition, the archive provides tertiary storage for the probe-pair data of the MER table.

In one embodiment of the present invention, the Archive may be organized as a multi-layered storage system. The first layer involves a relational database and a network file system, where the database maintains indices for fast content-based retrieval for the probe pair data, while the network file system stores the probe pair data and image data, such as the CEL and the DAT files, for the samples in DW. The second layer is based on a near-line optico-magnetic storage system that stores all data files as well as all the ancillary files generated by DMS, such as process tracking data, and intermediate data files. Generation of data files will be further described below with reference to the detailed description of DMS. The third layer of the archive is a second off-line back up storage system that provides enhanced recoverability and fault tolerance.

In accordance with an embodiment of the present invention, the Sample, Fragment Index, and Gene Expression databases of DW can be explored collectively or independently using an Explorer, which provides support for constructing gene and sample sets, for analyzing gene expression data in the context of gene and sample sets, and for managing individual or group analysis workspaces, such as User Workspace.

A Run Time Data Representation may also be provided to implement a multi-dimensional gene expression matrix (“GXM”) and rapidly access the core data stored in the DW. The multi-dimensional GXM may be used for exploring gene expression data and provides a data representation that is independent of the underlying gene expression technology platform. In one embodiment of the present invention, the data may include: absent/present calls for each sample/probe pair, intensities, and chips available for each sample. The run time data representation is part of the Run Time Engine, a system component that is intended to provide high performance gene expression analysis. In one embodiment of the present invention, programming access to Run Time Engine 260 may be through low-level C++ APIs to reflect the underlying implementation and memory model. In addition, high-level C++ APIs may be used to provide support for various high level concepts, such as gene sets and sample sets, which will be further described below. Moreover, an IDL interface based on high-level C++ APIs may be provided to support additional classes and methods necessary for performing high-level analysis functions.

The analysis methods supported by the Explorer and the Run Time Engine, provide an efficient mechanism to manipulate gene expression data. The middle layer of the computing architecture supports a range of APIs for integrating additional analysis tools. The list of the APIs includes a call-level interface to the gene expression archive (GXA), a query translator (middleware for database queries), and the Workspace API for user management.

In accordance with an embodiment of the present invention, the explorer supports a variety of analysis methods and tools. For example, one of the basic gene expression analysis operations provided by the present invention is the Gene Signature tool. The Gene Signature tool identifies consistently present and absent genes from a gene set, G, over a sample set, S. The result of a Gene Signature on G and S consists of the pair {CPG (G, S), CAG (G, S)}, where CPG denotes consistently present genes and CAG denotes consistently absent genes. A threshold, such as (card (5)−k), where card (S) denotes the cardinality of set S and k is 1, 2, . . . , n, is often used in computing Gene Signatures. A Gene Signature Differential analysis tool compares the results of two Gene Signature analyses and computes four new sets of fragments: those that are in both the first present gene set and the second absent gene set; those are in both the first absent gene set and second present gene set; those that are in both present gene sets; and those that are in both absent gene sets.

The accuracy of the Gene Signature depends on the size of the sample set, where a larger sample set ensures that genes that vary in expression between individuals are excluded. A Gene Signature over sample set S is considered accurate if adding any new sample to S reduces CPG (G, S)∪CAG (G, S) by no more than 2.5%.

Where CPG denotes consistently present genes, CAG denotes consistently absent genes, IPG denotes inconsistently present genes, and IAG denotes inconsistently absent genes. Let G be all the gene fragments monitored in DW and S a sample set. Present/Absence calls orders genes in G in four groups CPG, IPG, JAG, CAG. Gene Signatures analysis may be generalized to multiple sample sets, Si, . . . , Sn, as follows: Differentially expressed genes in set Si versus sets S2, . . . , Sn, defined by the pair:

{(CPG (G, S1)∩CAG (G, S2)∩ . . . ∩CAG (G, Sn)) (CAG (G, SI)∩CPG (G, S2)∩ . . . ∩CPG (G, Sn))}.

Unique consistently expressed genes in set S1 versus sets S2, . . . , Sn, defined by the pair:

{(CPG (G, S1)∩IPG (G, S2)∩ . . . ∩ IPG (G, Sn)), (CAG (G, SI)∩IAG (G, S2)∩ . . . ∩ IAG (G, Sn))}.

Common consistently expressed genes in S1, . . . , Sn, defined by the pair:

{(CPG (G, S1)∩ . . . ∩CPG (G, Sn)), (CAG (G, S1)∩ . . . ∩N CAG (G, Sn))}.

Common inconsistently expressed genes in S1 . . . , Sn, defined by the pair:

{(IPG (G, S1)∩ . . . ∩IPG (G, Sn)), (IAG (G, S1)∩ . . . ∩IAG (G, Sn))}.

Additional gene expression analysis operations supported by the explorer include fold change analysis and sample set analysis. Fold change analysis computes for each gene fragment in a get set G, the ratios of the mean log expression values between a sample set S and a control sample set; the first step of this analysis involves gene expression averaging on the sample dimension. Sample set analysis computes the range of expression levels for each gene in a gene set, G, across a sample set, S, in which the gene is consistently expressed. The first step of this analysis involves identifying the samples of a sample set in which all the genes from a gene set are consistently (present or absent) expressed genes.

Gene and sample query supports the definition of sample set and gene sets. Gene sequence query allows a user to determine if a gene sequence matches any of the genes or EST's in the Fragment Index Database.

Clustering allows to identify groups of similar genes or similar samples based on their expression profiles. This well-known technique is useful for learning the structure of a dataset without making any preconceived assumption.

Electronic northern tool analysis determines the ranges of expression values of genes and EST's across all tissue types represented in the DW. More particularly, a user-defined gene set and one or more samples sets are used to report the range of expression levels for each gene fragment in the gene set across each sample set, for all the samples where the fragment is called present. The range is reported using upper and lower percentile levels specified by the user. For example, if the user chooses 100% and 0% as the upper and lower percentile levels, the analysis reports the maximum and minimum range of expression levels for all present calls.

Results of gene expression exploration can be further examined in the context of gene annotations, such as pathway and chromosome maps, where gene expression data are represented in the framework of specific (e.g., metabolic) pathway or chromosome cytogenetic maps. A pathway visualization uses a graph representing the components of a metabolic or signaling pathway, highlighted with colored bands to denote the expression levels of the genes or gene products involved in the pathway. The bands may be divided horizontally into separate rectangles, each corresponding to an expression level for a particular sample. Alternatively, the pathway visualization may be used in conjunction with a fold change analysis, with the band colors corresponding to fold change values.

In a metabolic pathway, the components represent enzymatic activities that may be identified by EC numbers. Strongly and weakly expressed genes encoding enzymes are darkly and lightly shaded, respectively. Multiple genes may code for enzymes with the same activity, such as the many different alcohol dehydrogenases. In addition, multiple fragments may represent the same gene. The underlying pathway diagrams may be obtained from a public source, such as KEGG available at www.genome.ed.jp/kegg. Pathway visualizations may be performed for a particular sample set and gene set. The gene set may be computed indirectly from sample sets using the Gene Signature tool, Gene Signature Differential or Fold Change Analysis tools, or may be selected directly.

The results of gene data exploration can also be examined visually using third-party tools, such as Spotfire, marketed by Spotfire Corporation of Cambridge, Mass., or exported for analysis with statistical tools such as S-plus, marketed by Mathsoft Corporation of Seattle, Wash., GeneSpring from Silicon Genetics of San Carlos, Calif., Partek, etc.

Those skilled in the art should appreciate that the present invention may be implemented over a network environment. The network may be any one of a number of conventional network systems, including a local area network (“LAN”), a wide area network (“WAN”), or the Internet, as is known in the art (e.g., using Ethernet, IBM Token Ring, or the like). In addition, the present invention may also use data security systems, such as firewalls and/or encryption.

Having briefly described a suitable computing architecture in accordance with embodiments of the present invention, a more detailed description of the components of the architecture is set forth.

Data Warehouse

The data warehouse (DW) is provided to maintain very large amounts of data and has a structure that supports efficient gene expression exploration and analysis. In one embodiment of the present invention, DW is the integrated product of three component databases that materialize the sample, gene annotation, and gene expression data spaces discussed in the previous section. DW is loaded with sample, gene annotation, and expression data from a staging area where the data is integrated after passing data consistency and quality validation. The staging area may also have a transient database (not shown) that provides a buffer between the data sources of DW and DW while data undergo various transformations.

Sample database forms an independent data space for analytical processing. The fact object in the sample data space is a bio-sample representing the biological material that is screened in a microarray experiment.

A bio-sample has a type and a species. The type of a bio-sample can be tissue, cell line, processed RNA, etc., and originates from a species-specific (e.g., human, animal) donor. In one embodiment of the present invention, a human bio-sample is associated to one or more QC types of QC records completed by expert review. The pathology QC review documents the correct pathological processes represented on a given tissue. The image QC review documents any defects found on scanned image of a microarray chip. QC reviews are performed on every single fragment of a tissue sample.

A bio-sample may yield more than one genomic samples. A genomic sample is the entity screened in the production laboratory. A genomic sample might be based on

more than one fragment from a given sample so as to provide sufficient quantity to yield adequate RNA. Those skilled in the art should appreciate that in certain instances, such as samples from mouse organs, several bio-samples may be required to generate a genomic sample. If the bio-sample is of type RNA or IVT, then there is a one-to-one correspondence between the bio-sample and genomic sample.

In accordance with an embodiment of the present invention, samples may be associated with attributes that describe properties useful for gene expression analysis, such as sample structural and morphological characteristics (e.g., organ site, diagnosis, disease, stage of disease, etc.), donor data (e.g., demographic and clinical record for human donors, or strain, genetic modification, and treatment information for animal donors). Samples may also be involved in studies and therefore can be grouped into several time/treatment groups. More particularly, samples are related to other samples in ways that depend on the collection process and their respective studies. For example, some known forms of collection process sample relatedness include: explicitly matched samples-a tumor liver sample and a normal liver sample from the same excision; implicitly related samples—samples from the same donor without any connection to a common condition; sample series-ordered set of samples such as samples from early, middle, and late stages of disease progression; and time series-samples from a group of similar donors after being treated with a compound for 1, 6, and 24 hours respectively.

In addition, samples may be related to other samples through studies. One type of study provided by the present invention is a toxicology study, which is concerned with dose-response of samples/subjects overtime. Subjects, such as humans or rodents, are typically divided into multiple dose groups and observed at multiple time points. In rodent studies, bio-samples may be taken at sacrifice time as well as additional time points. Accordingly, a study may consist of many bio-samples grouped in groups of specific time and dose. A group may be seen either as a group of donors or a group of bio-samples.

Samples may be obtained from a variety of sources, with sample information structured and encoded in heterogeneous formats. Format differences range from the type of data being captured to different controlled vocabularies used in order to represent anatomy, diagnoses, and medication. In order to provide support for capturing samples from different sources, the sample data space is modeled as an independent data warehouse, with a star or snowflake schema structure, depending on the complexity of the sample data space. The sample category attributes can be organized in classification hierarchies implemented using controlled vocabularies or existing taxonomies such as the Systematized Nomenclature of Medicine (“SNOMED”) topography and morphology axes, for sample organ and diagnosis, respectively.

In accordance with one embodiment of the present invention, samples may be classified either as public or private samples. In other words, samples may be classified in terms of ownership of samples and their subsequently derived gene expression data. Ownership may be used for restricting access to the data generated by a sample. For example, samples may include alliance, project, and visibility attributes that define access to the information. For example, data from a sample may be visible by all or specific to the alliance that requested the information.

Gene fragment data, like sample data, may be considered as a separate data space shown as Fragment Index database. The fact object in the Fragment Index database is the gene fragment, representing the entity that is examined using a microarray. For example, for Affymetrix chips, the gene fragment represents the DNA sequence employed for synthesizing the oligonucleotide probes that are placed on the chips. Gene fragments are organized across two main dimensions: microarray design and biological annotation.

The microarray design describes the physical characteristics of a chip type design, including the placement of sequence fragments on the array. This information is provided by the microarray manufacturer and is used to interpret the signal in a microarray experiment. The biological annotation for a gene fragment comprises determining its biological context, including its associated primary sequence entry in public sequence databases such as Genbank, membership in a Unigene sequence cluster, association with a known gene in LocusLink, and functional and pathway characterization.

As those skilled in the art should appreciate, GenBank is the National Institutes of Health (“NIH”) genetic sequence database, an annotated collection of all publicly available DNA sequences that is available on the Internet at www.ncbi.nlm.nih.gov/Genbank. In addition, UniGene is a system for automatically partitioning GenBank sequences into a non-redundant set of gene-oriented clusters and is available at www.ncbi.nlm.nih.govfUniGene/. Finally, LocusLink provides a single query interface to curated sequence and descriptive information about genetic loci and is available at www.locuslink.com. LocusLink presents information on official nomenclature, aliases, sequence accessions, phenotypes, EC numbers, MIM numbers, UniGene clusters, homology, map locations, and related web sites.

Gene fragment annotation involves integrating information from a variety of genomic data sources.

An important aspect of the Fragment Index database is the evolution of the science underlying recorded gene annotations. For example, the association of a gene fragment to a known gene may change because of the evolution of Unigene clusters or amendments to the known gene entries recorded in LocusLink. The evolution of gene data may affect the result of gene expression data analysis, and therefore must be tracked. The reader should appreciate, however, that gene data changes are different from historical data changes in traditional data warehouses in that historical data changes typically record changes of known indisputable facts (e.g., prices of products) while the evolving gene data changes record changes in what is known about scientific facts. Accordingly, gene annotation and gene sequence data must not only be extracted, validated, and integrated into DW, but also refreshed to reflect the evolution of science.

OLAP-like operations can be used for navigating the Fragment Index database mainly along the biological annotation dimension. For example, examining gene fragments associated with metabolic pathways may involve a selection of metabolic pathways and a projection on the pathway dimension. More particularly, in a classification of gene annotation data using the following hierarchy: Species to Chromosome to Known Gene, summarization of the gene fragments on known genes would result in the total number of fragments classified by their association with a known gene; further summarization on chromosome would result in the total number of gene fragments classified by chromosome.

Gene expression data, like gene annotation and sample data, may also be considered as a separate data space such as Gene Expression database. Gene expression data may comprise data generated using READS technology, marketed by Gene Logic Corporation of Gaithersburg, Md., and QPCR technology, marketed by Lark Technologies Corporation of Houston, Tex. Those skilled in the art should appreciate that gene expression data originating from different platforms may be managed and structured independently, rather than using a common data format. Gene expression data generated using different platforms may be correlated via common samples (i.e. samples that are run using different technologies) or common genes.

The multi-dimensional GXA used for exploring gene expression data provides a data representation that is independent of the underlying gene expression technology platform. Thus, the GXA can be used for uniformly exploring gene expression data generated using diverse platforms, such as the GeneChip, READS, QPCR, and cDNA Microarray platforms. The GXA provides the framework for implementing the gene expression operations described above, and for integrating advanced data mining algorithms.

The fact object in the gene expression data space is the gene expression value. Gene expression data may be defined at several granularity levels. The data generated by measurement instruments, such as scanners, are at the highest level of granularity. Analysis programs turn the data into quantitative gene expression measurements. For example, the Affymetrix GeneChip involves (a) a cell averaging step that averages pixel intensities and computes cell-level intensities, where each cell corresponds to one probe on the microarray, followed by (b) a chip analysis step that generates gene expression values by “summarizing” the intensities of approximately 20 probe pairs that correspond to each gene or EST fragment on the microarray. The GeneChip expression value consists of a presence/absence (“PA”) call and an absolute gene expression measurement. Alternate platforms, such as QPCR, reports an expression value per gene and per sample, relative to a reference sample. The present invention provides a multi-dimensional structure that supports representing gene expression values generated with different platforms or analysis methods.

The four primary dimensions in the gene expression data space are gene, sample, method and experiment, where gene and sample provide the connection to the gene annotation and sample data spaces.

In one embodiment of the present invention, the experiment dimension links gene expression data to parameters such as the chip lot, experimental protocol, and software version. These parameters refer to the data generation process.

The method dimension models the different gene expression values generated using different analysis methods, such as GeneChip PA values and GeneChip generated absolute gene expression values. Gene expression values can be classified into present, absent, marginal, or unknown calls.

Variants of OLAP operators may be used to define basic operations in the gene expression data space, which can then be used to define more complex data analysis operations.

For example, in a simplified gene expression data space with three dimensions: sample, gene, and expression measure type, a valuation function, v, may be defined that returns the expression value of a gene, g, and sample, s. Where the expression measure type, E, is either E_{PA} or E_{Abs}, E_{PA} measurements are either present, p. absent, a, or marginal/unknown calls, m, and E_{Abs} measurements are absolute gene expression values, then: v (g, s, p) may be defined as “1” if g is associated with a present call for s in E_{PA} and “0” otherwise; v (g, s, a) may be defined as “-1” if g is associated with an absent call for s in E_{PA} and “0” otherwise; v (g, s, x) may be defined as “1” if g is present in s, “-1” if g is absent in s, and “0” otherwise; and v (g, s, abs) may be defined as the absolute gene expression value for g and s in E_{Abs}.

In addition, sample selections may be defined over the sample data space in order to extract sets of samples with a certain profile. For example, a sample set may consist of male colon samples with adenocarcenoma from donors in the age group 40-60 that do not have a smoking history.

Likewise, gene selections may be defined over the gene annotation data space in order to extract sets of genes with certain properties. For example, a gene set may consist of the genes on chromosome 22 whose protein products are involved in the estrogen metabolism pathway. Gene and sample sets may be used in gene expression operations discussed below.

Those skilled in the art should appreciate that analyzing gene expression data over arbitrary sets of genes and samples may not be biologically meaningful. For example, analyzing gene expression across samples from different species may not yield biologically meaningful results. Consequently, gene and sample operations may need to be restricted in order to ensure that the resulting sets are consistent from a gene expression analysis point of view.

Furthermore, those skilled in the art should also appreciate that a gene expression summarization function can be defined over the entire sample and gene set dimensions or a set of genes and a set of samples, where the sample set has been specified using a sample selection and the gene set has been specified using a gene selection.

Gene expression summarization on the sample dimension summarizes for each gene in the gene set, the gene expression measures over the samples in the sample set. For example, given a gene set, G, and sample set, S, the gene expression summarization on S, results in expression summary σ (g, e, S), for each gene g in G, and each e in EPA. Summary σ (g, e, S) consists of the sum of expression measures over all samples of S for each pair g and e, i.e., σ (g, e, S)=Sum [v (g, sᵢ, e)|sᵢ in S].

Gene expression summarization on the gene dimension summarizes for each sample in the sample set, the gene expression values over all genes in the gene set. For example, given a gene set, G, and sample set, S, the gene expression summarization on G, results in expression summary σ (s, e, G), for each sample s in S, and e in EPA. Summary σ (s, e, G) consists of the sum of expression measures over all genes of G for each pair s and e, i.e., σ (s, e, S) =Sum [v (gᵢ, s, e)|gᵢ in G].

Gene expression averaging on the sample dimension averages for each gene in the gene set, the absolute gene expression values over the samples in the sample set. For example, given a gene set, G, and sample set, S, the gene expression value averaging on S, M (G, S), results in the set of mean expression values, μ (gᵢ, S), for each gene gᵢ in G, that is, M (G, S)={μ(gᵢ, S)|μ(gᵢ, S) mean [v (g, sⱼ, abs) sⱼ in S], gᵢ in G}.

Having briefly described some basic operations using variants of OLAP operators, more complex data analysis operations may be defined. More particularly, consistently expressed gene operations may be defined over a set of genes and a set of samples to define the set of consistently present and consistently absent genes in a sample set.

For example, in a given gene set, G, and sample set, S, the sets of consistently present (“CPG”) and consistently absent (“CAG”) genes in S, may be defined as follows:

CPG (G, S)={gi|σ(gᵢ, p, S) card (S) and gᵢ in G}; CAG (G, S)={gᵢ|−σ(gᵢ, a, S)=card (S) and gᵢ in G}.

The set of inconsistently expressed genes (“IEG”) may then be defined as:

IEG (G, S)=G−CPG (G,S)−CAG (G,S).

Those skilled in the art should appreciate that sets CPG (G, S), CAG (G, S), and IEG (G, S) partition the set of genes G with regard to the way genes are expressed in sample set S. In other words, the sets are pair-wise disjoint. Other operations can be defined using the CPG, CAG, and IEG operations, particularly IPG (G, S), defining the genes that are inconsistently present in S, and IAG (G, S), defining the genes that are inconsistently absent in S. For example, IPG (G, S)=IEG (G, S)∪CAG (G, S); IAG (G, S)=IEG (G, S)∪CPG (G, S).

Similar operations may define the subset of samples in which the genes from a given gene set are either all present or all absent in a given sample set. For example, in a given gene set, G, and sample set, S, the subsets of samples of S in which all the G genes are consistently present (“CPS”), consistently absent (“CAS”), or inconsistently expressed (“IES”) may be defined as follows:

CPS (G, S)={sᵢ|(sᵢ, p, G)=card (G) and sᵢ in S};

CAS (G,S)={sᵢ|−σ(sᵢ, a, G)=card (G) and sᵢ in S}; and

IES (G, S)=S−CPS (G, S)−CAS (G, S).

In one embodiment of the present invention, the CPG, CAG, CPS, and CAP operations may be varied using an additional threshold, T, for defining the gene expression consistency in terms of the minimum number of samples out of the total number of samples in 5, for which the genes are present or absent.

In addition, derived operations can be used to contrast expressed genes in a set of samples with expressed genes in another set of samples. For example, in a given gene set, G, and sample sets, S1 and S2:

for differentially expressed genes in set S1 versus set S2:

CPG (G, S1)∩CAG (G, S2)

defines the set of G genes that are consistently present in samples of S1 and consistently absent in samples of S2; and

CAG (G, S1)∩CPG (G, S2)

defines the set of G genes that are consistently absent in samples of S1 and consistently present in samples of S2;

for unique consistently expressed genes in set S1 versus set S2:

CPG (G, S1)∩IPG (G, S2)

defines the set of G genes that are consistently present only in samples of SI (i.e., not consistently present in samples of S2); and

CAG (G, S1)∩IAG (G, S2)

defines the set of G genes that are consistently absent only in samples of S1;

for common inconsistently expressed genes in S1 and S2:

CPG (G, S1)∩CPG (G, S2)

defines the set of G genes that are consistently present both in samples of S1 and in samples of S2; and

CAG (G, S1)∩CAG (G, S2)

defines the set of G genes that are consistently present both in samples of SI and in samples of S2; and

for common inconsistently expressed genes in S1 and S2:

IPG (G, S1)∩IPG (G, S2) defines the set of G genes that are inconsistently present both in samples of S1 and in samples of S2; and

IAG (G, S1)∩IAG (G, S2) defines the set of G genes that are inconsistently present both in samples of S1 and in samples of S2.

Gene and sample correlation operations can be defined over a set of genes and a set of samples after gene expression summarization on gene expression value type has been applied on the gene expression data space 226. Gene correlation can be defined using a similarity, or distance, measure. The similarity of two genes, g1 and g2, over a sample set S, is measured by the sum of |v (s, g1, x)−v (s, g2, x)| over all the samples of S. Accordingly, genes g1 and g2 are similarly expressed in S, if v (s, g1, x)=v (s, g2, x) for each sample s of S.

Those skilled in the art should appreciate that gene and sample correlation can similarly be used in grouping, or clustering genes and samples based on their similarity.

Having briefly described the Data Warehouse in accordance with embodiments of the present invention, a more detailed description of Data Management System is set forth.

Data Management System

In accordance with an embodiment of the present invention, gene expression data may be generated in a high throughput production environment using Affymetrix GeneChip technology and READS proprietary differential expression profiling technology. QPCR may also be used to validate GeneChip and READS results.

Large-scale data processing requires data management facilities for acquiring, organizing, managing, integrating, and exploring massive amounts of data.

In accordance with an embodiment of the present invention, DMS comprises operational databases and LIMS applications that support data acquisition and management of production data.

DMS provides support for various sample acquisition and quality control protocols, via data entry, data migration, and reporting tools. The system uses domain specific vocabularies and taxonomies, such as SNOMED, to ensure consistency during data collection, and records the data in a database with a structure that is compatible with sample data space.

In addition, DMS provides support for high-throughput for Gene Logic's Affymetrix-based gene expression production and seamless integration with the Affymetrix GeneChip LIMS.

DMS manages gene expression experiment, QC/QA, and process data. In one embodiment of the present invention, gene expression experiment data generated by the GeneChip system are provided in files in Affymetrix proprietary formats: (a) a binary image of a scanned microarray is contained in a DAT file; (b) the DAT file is converted to a CEL file using a cell averaging analysis operation that generates average intensities for the probes on the microarray; and (c) the CEL file is converted into a CHP file by a chip analysis operation that generates the expression values of gene fragments probed in the microarray. Finally, the GeneChip LIMS supports a publishing operation that turns the CEL and CHP files and process data into a relational representation based on the AADM schema and stores it in a transient database.

DMS integrates seamlessly the sample data management system with the GeneChip LIMS and a Chip QC module, thus ensuring data consistency across and efficient data flow through component data management systems. The Chip QC component is used for detecting chip image defects using both image software and manual visual analysis and for masking the probes affected by these defects. Furthermore, DMS accelerates the rate of data generation by providing support for parallel publishing via multiple GeneChip LIMS systems.

In accordance with one embodiment of the present invention, DMS directs the data generated by the GeneChip LIMS as follows: the DAT, CEL, CHP files are sent to the archive; the gene expression data, in relational AADM format, and the QC data are transferred to the DW staging area where the necessary data integration, transformation, validation, and correction are performed before loading the data into DW. For example, in accordance with one embodiment of the present invention, consistency checks may comprise: matching filenames to sample names; matching filenames to array types; preventing duplicated data; checking tissue type against a controlled vocabulary, such as SNOMED; checking that the CHP file contains the correct list of genes; checking that the number of cells are correct; and checking that no relative data is included.

Data management for READS and QPCR gene expression data may be provided by Gene Logic proprietary systems. READS and QPCR data are represented in a high-level object model and are stored in relational databases. READS and QPCR files are also archived, while the data in relational format are transferred to the DW staging area where they are handled in the same way as GeneChip data.

Although a few specific embodiments of the present invention have been described in detail, it should be understood that the present invention may be embodied in many other specific forms without departing from the spirit or scope of the invention as recited in the claims.

The present invention pertains to relational databases for storing and retrieving biological information comprising an integration of at least three databases organized to support exploration and mining of gene expression data. The at least three databases include: (1) a gene expression database storing quantitative gene expression measurements for tissues and cell lines (from hereafter both are termed bio-samples) screened using various assays; (2) a clinical database which stores information on bio-samples and donors; and (3) fragment index is a comprehensive database of biological properties (annotations) for all fragments (full length genes and EST's).

In a preferred embodiment of the present invention, the gene expression database for storing quantitative gene expression measurements from tissues and cell lines are screened using Affymetrix human, rat and mouse micro-arrays. It will be appreciated that the information in the gene expression database can preferably organized so as to meet specified quality control criteria and functional specifications.

In a preferred embodiment of the present invention, the bio-sample specific information stored by the clinical database includes pathology, diagnosis, accrual and treatment facts. Donor information includes donor demographics, clinical histories for human donors and laboratory tests for animal models. Clinical data are recorded using standardized vocabularies compliant with established nomenclatures such as SNOMED.

In a preferred embodiment of the present invention, the fragment index is a comprehensive database of biological properties (annotations) for all fragments (full-length genes and EST's) on the Affymetrix gene expression micro-arrays. Fragment annotations preferably include association to genes in the official HUGO nomenclature, links to related entries in public databases, and phenotype, structure, function and pathway information retrieved and digested from the public databases.

The key objective of the relational database for storing and retrieving biological information of the present invention is to provide comprehensive access to gene expression and support for biological analysis. In the architecture of the present invention, these objectives are obtained by the query capabilities that the relational databases of the present invention provide, as well as an application server that supports a biology-meaningful online analytical processor of the database data. This biology-meaningful online analytical processor examines large scale gene expression analysis of the data found in the relational database for storing and retrieving biological information so as to reveal gene expression patterns that characterize certain functional states of the physiology of an organism. Operations supported by the application server include filtering, clustering, summarization, comparison and mapping onto pathways of gene expression data.

The functionality of the relational database for storing and retrieving biological information including its application server, is presented to a user via the relational database user interface. In a preferred embodiment of the present invention, the relational database user interface is provided in two formats, the first as a web application and the second as a Java client application.

The relational database for storing and retrieving biological information, the application server, a client side user interface and a user's workspace database, preferably define a three-tier architecture to gene expression data and analysis. In a preferred embodiment, this system is integrated with an archive, an external file system that stores experimental data files and data for all experiments in the relational database for storing and retrieving biological information.

The relational database for storing and retrieving biological information is the repository of gene expression data produced by a genomics production pipeline. A relational database management system is the backbone data management infrastructure that supports the data flow of the production pipeline. The relational database management system is a complex, distributed heterogeneous system whose main components are interfaced by software modules enforcing well-defined protocols.

The main components, preferably, of the relational database management system are: (1) a relational database management system; (2) a genomics production sample tracking system; (3) an application that documents the processes that generate the experimental files; (4) a software module that turns experimental files into a relational representation; and (5) a defect-inspecting software module.

In a preferred embodiment of the present invention, the tissue repository information management system is an information system that supports the production cycle of a bio-repository, which support includes accessioning and inventory management of bio-samples, inputting pathology assessment and clinical data, and exporting of clinical data to the relational database for storing and retrieving biological information.

In a preferred embodiment of the present invention, the genomics production sample tracking system consists of a collection of spread sheets which track samples as they move along the production pipeline. In another preferred embodiment of the present invention, the application that documents the processes that generate the experimental files relates to the DAT, CEL and CHP files for each experiment. This process documentation is preferably stored in an Affymetrix database. This application minimizes data entry overhead.

In a preferred embodiment of the present invention, the software module that turns experimental files into a relational representation supports several parallel publishing engines and also performs a list of consistency checks to ensure that the production standard operating procedure and publishing processes were executed successfully. This software module also preferably dumps the individual databases into text files (per table) and transfers them to a designated area in a staging UNIX server.

In another preferred embodiment of the present invention, the defect-inspection module is a semi-automatic process in which chip images (DAT files) are inspected for defects that affect the quality of generated expression data. The result of this process are quality control reports, one per experiment, that are also migrated to the staging UNIX server.

The totality of these data streams defines the interface between the relational database management system and the relational database for storing and retrieving biological information. Specifically, all these data streams feed into a staging area where a warehouse building processes take place, i.e., validation, transformation and integration of the data.

The migration of data from the various data sources to staging is controlled by data migration protocols. In a preferred embodiment of the present invention, these data migration protocols include an expression data migration protocol; a tissue repository information management system for clinical data; and a chip-defects migration protocol.

The expression data migration protocol, preferably, includes daily publishing documented by an email report; publishing data (per publishing engine) by dumping into TXT files (one per each gene expression data table) and a LST file; verifying line counts of the TXT files; copying files to pre-staging (an incoming directory on the UNIX server) by an ftp process; notification by the publishing operator to the staging DBA that the ftp process is done upon completion of the ftp process; verification by the staging DBA of the line count of files; loading to staging concluded with a loading report emailed to the relational database for storing and retrieving g biological information; and staging protocol triggers with 1 day (24 hrs) from the loading time.

A preferred embodiment of the present invention utilizes data integration, a process of bringing together experimental data generated by parallel and independent publishing processes. Parallelism in publishing is introduced to satisfy high-throughput requirements and to permit generation of experimental data files in different facilities.

This data integration serves to scan and validate AADM published data and to adjust identifiers generated by parallel publishing processes in a sequential order. this data integration is extensible, in the sense that process specific validation rules can be added and enforced by the system.

In another preferred embodiment of the present invention, gene expression I integration is also provided. Gene expression integration refers to the integration of experimental data with clinical and public gene data (Fragment Index). Gene expression integration is a task performed at the staging database.

The present invention is further characterized by a database schema. This schema itself can preferably be divided into four related sub-schemas: (1) probe array design; (2) experiment setup; (3) analysis results; and (4) protocol parameters.

With regard to probe array design, this part of the schema holds data describing a probe's array physical and biological design. The most important part in this sub-schema, is the association of biological items (gene fragments) to blocks in a particular probe array type. Probe array types are recorded in the PROBE_ARRAY_DESIGN table. A PROBE_ARRAY_DESIGN instance describes the physical layout of an expression chip type. PROBEARRAY_DESIGN is related via the ANALYSIS_SCHEME relationship to a SCHEME_UNIT entity. Although, the general design goal in data integration is to be able to attach several “logical” designs to a physical chip design, in the case with expression probe arrays there is a one-to-one relationship between physical and logical design. This translates to a one-to-one correspondence between SCHEME UNITS and SCHEME BLOCKS. Each block interrogates a single gene fragment. A block unit is divided into atoms. In gene expression probe arrays, an atom consists of two cells. Each cell corresponds to 25-mer oligonucleotide probe. A block representing a gene fragment consists of approximately of 20 probe pairs, each probe pair corresponding to an atom with a perfect match and a mismatch probe cells.

The AADM probe array design sub-schema contains parts that are not used/needed in any gene expression exploration queries. The intention for this sub-schema was to hold a variety of Affymetrix probe array designs and therefore is used the Affymetrix analysis software to relate probe intensities to biological items.

The experiment setup sub-schema holds information on the probe arrays used and the target applied in any gene expression experiment. An EXPERIMENT is the event during which a physical chip and a target are “joined”. As the target is applied on a chip probes of the chip hybridize with gene regions of the target. The chip surface is scanned to generate a DAT file where the hybridization result is permanently printed. Subsequently the DAT file is analyzed in order to extract useful biological data. An experiment is controlled by a protocol. A protocol dictates how the experiment should be conducted and which captures administrative information and data about the environmental conditions during the experiment. The database, by capturing a record (or object) per experiment run, enables the association between experimental results, tissues that are processed into targets, and resulting datasets (via the DAT).

A TARGET is prepared out of a bio-sample and therefore is the connecting entity between experiments and sample specific information. This association in AADM is very limiting since it only supports one parameter to describe the target and this is the TARGET_TYPE.

A PHYSICAL_PROBE_ARRAY (chip) is the physical apparatus used to carry out the hybridization and scan experiment. A physical chip is identified by a serial number, belongs to a particular probe array design and has an expiration date.

The analysis results sub-schema stores results from various analyses, including cell averaging, absolute gene expression and comparative gene expression analysis. It is preferred to use cell averaging and absolute gene expression analyses, only.

The analysis process works as follows. A hybridization/scan experiment generates an image file, call the DAT file. The DAT file is analyzed and the its quantitative representation, the CEL file, is generated. This analysis is called cell analysis. Cell analysis first fits a grid to separate the cell (which correspond to probes) of the image and second calculates the average intensity value for all pixels in a cell. In AADM the results of cell analysis are stored in the MEASUREMENT_ELEMENT_RESULT table (MER for short). A subsequent analysis step, called chip analysis, performs “expression calling” on the CEL file. The result of this process is an assertion of gene expression of all gene fragments on the chip that includes the average intensity and a presence/absence (P/A) call. The results of the chip analysis are stored in the ABSGENE_EXPR_RESULTS table (AGER for short). The ANALYSIS table in the schema stores an analysis record for any analysis performed. An analysis record is identified by an analysis id (key) and is related to: the protocol used for the analysis, an analysis scheme (and transitively a chip type), the algorithm, analyst and the dataset on which the analysis is performed. An analysis record also stores the date and a name for the analysis.

Input data set(s) to analysis are recorded in the ANALYSIS_DATA_SET table. Data sets are grouped in collections of data sets. AADM uses the ANALYSIS_DATA_SET_COLLECTION table to unsuccessfully model a many-to-many relationship between analyses and analysis data sets ANALYSIS_DATA_SET stores a record for each type of analysis, i.e., cell analysis and chip analysis. In cell analysis the input data set is an experiment (DAT file). In chip analysis the input data set is an analysis. With regard to the protocol parameters, this sub-schema contains parameters captured during, the experiment setup, hybridization experiment, and cell and chip analyses. The data in this sub-schema are essential for the production and quality control groups who want to track the data generating processes. The relational database for storing and retrieving biological information also uses values of certain protocol parameters, such as the version of the production standard operating procedure, in order to partition expression data into meaningful and comparable subsets.

In a particularly preferred embodiment, the present invention provides a staging database. This staging database is an area where several warehouse building processes take place. The staging database is, preferably, an Oracle database running on a UNIX server which also functions as the pre-staging area where several ftp processes deposit data produced by the data management tool.

In utilizing such a staging database, it is preferable to run a staging protocol. In such a staging protocol expression data in staging are processed and transformed. The staging protocol is a routine of steps that are performed each time expression data are loaded from pre-staging into the staging database. The staging protocol expects that expression experiments are named according to the nomenclature defined in the publishing SOP version 3.0. Preferably, a valid experiment name is a 13 characters long string, nnnnncccccccsr, where

| | |
|---|---|
| nnnnn | is the 5 digits genomic number (e.g., 00231) used by production |
| | to track a sample |
| cccccc | is a six character string that represents the chip type used in the |
| | experiment, e.g., Hu35KA |
| r | is a single digit number representing the repetition count that the |
| | same genomics sample has been hybridized on a chips |
| | is a single digit number representing the scans count of the same |
| | chip |

The staging database permits extensions to allow the management of other specific practices not identified above. For example, the passage of experiments through staging can be tracked using the GLGC_EXPERIMENT table. The steps that the staging protocol takes depend whether production does a single or double scan per chip. In the case of double scans, the staging protocol classifies the scan into a primary and a secondary, consolidates the expression presence/absence calls of the secondary into the primary and migrates the primary into the warehouse.

Another optional step of the staging protocol depends on the type of probe pair generated during this process. One option is to generate “digested” probe pair data containing the probe-level cell intensities as well as the summarized expression call of all probes per an Affymetrix gene fragment. The second option is to simply store cell intensities of probes per experiment into separate comma delimited text files. The steps of the staging protocol are: (1) export and backup the staging database; (2) check consistency of data files in the incoming directory; (3) load data into the data integration tables; (4) update the GLGC_EXPERIMENT table; (5) compute the rank (primary/secondary) of experiments with multiple scans; (6) consolidate primary and secondary experiments; (7) migrate primary experiment data into the relational database; (8) generate the “digested” probe pair data; (9) delete migrated data; (10) generate statistics about the staging activity; and (11) export and backup the staging database. Steps 1, 2, 3, 4, 7, 9, 10 and 11 are compulsory. Steps 5 and 6 refer to the double scan situation. Step 8 applies only if “digested” probe pair data are calculated, otherwise plain probe pair data are generated in step 2.

The experimental data migrated to the relational database are the summarized expression calls per gene fragment, i.e., the AGER table, and not the probe intensities, the MER table. The probe intensities are stored in text files named by the experiment name and directed to the archive.

Another important function of the staging database is expression data integration, i.e., linking the expression data with the clinical database and the fragment index. Although these data will physically “get together” in the relational database, the staging database adds this capability. Specifically, for clinical data, it decodes the experiment name and extracts the genomics sample number out of it. This number is associated with the bio-repository id and hence the sample and clinical information, through the BIO_{—}2_GEN table exported by the production tracking system. Table GLGC_EXPERIMENT associates the genomics number to the ANALYSIS_ID for both the cell and chip analyses performed to this experiment, then a referential integrity constraint ensures that the corresponding data records exist in the AGER and MER tables. The constraint to the MER table is disabled in GXDB, because MER data are not available.

Fragment index integration is a task directly done in the relational database. The fragment index, by design, maintains a list of gene fragments, a.k.a. items, exactly in the same order as the items in the AADM BIOLOGICAL_ITEM table. The addition of a foreign key constraint from AGER to the fragment index AFFY_ITEM table, provides for integration.

Additional integration tasks include the masking of defective gene fragments on chips out of experimental data and enforcement of the sample completion constraint. The chip quality control identifies defective spots in the scanned images that should not be incorporated in cell and chip analyses. The quality control process reports the gene fragments per experiment that are affected by image defects, in files that are transferred to the pre-staging area. These files are used to mask out expression data points by turning the Present/Absent (P/A) call to Unknown (U). The old P/A called is saved and can be restored anytime the quality control report is reverted.

Working with chips grouped in sets, such as the Human 42K set, requires running the same genomic sample over several chips. In order to complete a vector of 42K expression data points for each sample, data from all 5 chips need to be in the database. The process of getting all chips per sample in order to make a complete expression vector is called sample completion. A preferred embodiment of the present architecture allows enforcement of sample completion at staging, at the relational database, or not at all.

In a preferred embodiment of the present invention, during loading, data are checked for consistency. The consistency rules preferably applied are a subset of the rules checked in publishing before the migration to pre-staging. The following rules are preferably applied per experiment/chip basis.

| Rule name | Description |
|---|---|
| name consistency | verifies whether the experiment name complies |
| | with the naming nomenclature. |
| chip type consistency | verifies whether the chip type name component of |
| | the experiment name is one of the chip type |
| | names in the controlled vocabulary (e.g., |
| | Hu35KA) and the corresponding Affymetrix chip |
| | type name (e.g., Hu35KsubA) exists in the |
| | database. |
| cell cardinality | checks whether the number of rows in the MER |
| | table matches the expected number of rows for |
| | the same chip type. |
| gene cardinality | checks whether the number of rows in the AGER |
| | matches the expected number of rows for the |
| | same chip type. |
| chip correctly analyzed | A chip that is analyzed, for instance, as |
| | Hu35KsubA must be of type Hu35KsubA. |
| correct project name | the project name of the experiment must be part |
| | of the experiment names controlled vocabulary. |
| mask consistency | verifies whether the proper mask library |
| | (specified in the SOP) has been applied during |
| | analysis. |
| chip already loaded | the same chip experiment has already been loaded |
| | in staging |
| correct genomic id | verifies that the genomics sample number |
| | registered in experiment setup matches the |
| | genomics sample number in the experiment name. |
| correct SOP version | verifies that the production standard operating |
| | procedure entered is the standard operating |
| | procedure in effect for the date/time of the |
| | experiment |
| valid target type | verifies that the target type value is a valid tissue |
| | type. |
| valid dates | Dates in EXPERIMENT and ANALYSIS tables |
| | are earlier than current date, but not more than |
| | six months apart. ANALYSIS date is later than |
| | EXPERIMENT |

In another preferred embodiment of the present invention, the staging database is a proper relational database with SQL query capability. The staging database preferably also provides reports to track the staging activity. Such reports include a staging loading eport, issued any time loading to the staging database occurs; a staging weekly report which reports the staging activity per week, i.e., number of experiments loaded in, number of experiments migrated to the relational database, etc.; and a staging weekly exception report which reviews double scan experiments, and reports the experiment names of experiments waiting for the “mate” scan (are on hold) for longer than 5 days.

In another preferred embodiment of the present invention the relational database provides extensions to support the Gene Express process model. List of AADM tables

ABS_GENE_EXPR_ATOM_RESULT
ABS_GENE_EXPR_RESULT
ABS_GENE_EXPR_RESULT_TYPE
ALGORITHM_TYPE
ANALYSIS
ANALYSIS_ALGORITHM
ANALYSIS_DATA_SET
ANALYSIS_DATA_SET_COLLECTION
ANALYSIS_DATA_SET_TYPE
ANALYSIS_SCHEME
BIOLOGICAL ITEM
CHIP_DESIGN
EXPERIMENT
MEASUREMENT_ELEMENT_RESULT
PARAMETER
PARAMETER_TEMPLATE
PARAMETER_TYPE
PARAMETER_UNITS
PHYSICAL_CHIP
PROTOCOL
PROTOCOL_TEMPLATE
REL_GENE_EXPR_RESULT
REL_GENE_EXPR_RESULT_TYPE
SCHEME_ATOM
SCHEME_BLOCK
SCHEME_CELL
SCHEME_UNIT
TARGET
TARGET_TYPE
TEMPLATE_TYPE
UNIT_TYPE

An aspect of the present invention is ensuring the data integrity of the data in the relational database for storing and retrieving biological information. Database referential integrity maintains the relationships of the data modeled in the database schema. Various application-specific rules and general biological rules need to be constructed in the data. This is accomplished by identifying the application-specific rules and general biological, translate the application-specific rules and general biological represent rules into PL/SQL functions, and store the resultant functions in a rule base within the relational database for storing and retrieving biological information. It will be appreciated that these application-specific rules and general biological functions will periodically be run by the relational database rule engine to ascertain the accuracy and integrity of the data stored in the relational database.

It will be appreciated that there are several application-specific rules and general biological rules appropriate for use with the relational database for storing and retrieving biological information. Exemplary rules include chip consistency rules; chip defects report consistency rules; clinical data/gene expression data consistency; Fragment/gene expression data consistency rules; and expression integrity rules.

Chip consistency rules assess the microarray for consistency and are preferably checked at the time of publishing and data staging. Chip defects report consistency rules assess the chip defects report for consistency. For example, the gene fragment names in the chip defects report per experiment should match the gene fragment names of the chip type in the experiment. Clinical data consistency rules assess the internal consistency of the clinical data. Clinical data/gene expression data consistency assess the consistency of the clinical data with the gene expression data. For example, the organ name in the clinical database should match the target type value in the gene expression data for the same sample. Matching is preferably performed at variable granularity, i.e., organ “cerebellum” matches target type “brain”. Fragment/gene expression data consistency assesses the consistency of the fragment index data with the gene expression data. Preferably, this rule verifies that the ID and ITEM_NAME in BIOLOGICAL_ITEM joined with the ANALYSIS_SCHEME.ID, matches the ITEM_ID, AFFY_NAME and ON_CHIP attributes of the fragment index's AFFY_NAME. Expression integrity rules are based on biological knowledge. For example, if a gene is known to be present in a specific tissue type, then it should be present in the relational database. Special classes of this rules handle the housekeeping (or spiking) genes for which there is prior knowledge as of whether they are present or absent. The application-specific rules and general biological rules are organized by modules, and are stored in the Rule Repository. When an application-specific or general biological function is run and an error is detected, then the system generates an error codes and/or corrects the error by means of the error engine. In addition, a log and audit engine creates a log and audit of the run.

Although the relational database for storing and retrieving biological information accepts data by experiment, the user preferably views data by sample. In a preferred embodiment a user has a restricted view of samples, based on ownership and authorization. Data in the relational database for storing and retrieving biological information are preferably organized by partitions, access rights. Furthermore, data partitions may be cloned out of the relational database into separate, smaller access group-specific databases. A sample data vector in the relational database refers to all the data attributed to a sample, e.g., for the Human 42K a sample data vector would contain all the 42K data points that are generated in 5 chip experiments. Because there can be several runs on the same sample, there can be several data vector candidates in the relational database per sample. One such scenario is listed in the table below where genomics 00012 has 3 possible data vectors

| Experiment Name | Data Vector Candidate Group Identification |
|---|---|
| 00012Hu35KA12 | 1 |
| 000i2Hu35KB12 | 1 |
| 00012Hu35KC12 | 1 |
| 00012Hu35KD12 | I |
| 00012Hu35KE12 | 1 |
| 00012Hu35KA22 | 2, 3 |
| 00012Hu35KB22 | 2, 3 |
| 00012Hu35KC22 | 2, 3 |
| 00012Hu35KC32 | 2, 3 |
| 00012Hu35KD32 | 2, 3 |
| 00012Hu35KE32 | 2, 3 |

Partitioning is the process by which sample data vectors are segregated according to partitioning schemes or partitioning types. For example, sample data vectors can be partitioned according to project, tissue normality (diseased or normal), organ, collaboration, etc. Partitioned sample data vectors can restrict access to specific users.

The construction of primary data vectors per sample is done automatically using heuristic rules defined by production, or by manually overriding the automatic grouping. For example, if more than one chip of each type, e.g., two A chips, are available per sample, the one with the higher run number goes into the primary vector. The experiments groups defining sample data vectors are stored in a table EXPERIMENT_GROUP.

| | | | | |
|---|---|---|---|---|
| GROUP_ID | EXPERIMENT_ID | STATUS | MASK | CMASK |

Attributes MASK and CMASK are used for partitioning. Their values are based on the partitioning properties for a given sample. The CMASK attribute is used for filtering the data for requests from a user and the MASK attribute is a numeric value that can be used for physically partitioning (Oracle 8 partitions) the schema. When a sample should not be in a particular partition, these attributes take default values that make the sample data vector a component of the global partition. This is best understood with the help of examples. The following example illustrates how possible partitioning variables with their values and a numeric code are used to form parts of the mask.

| Collaborator | Project | Organ | Normality |
|---|---|---|---|
| JT I | HPR 1 | Heart 1 | Malignant 1 |
| P&G 2 | HRD 2 | Liver 2 | Normal 2 |
| . . . | MPR 3 | Kidney 3 | . . . |
| . . . | MRD4 | . . . | . . . |

Let N be the total count of values for an attribute, let genomics 00120 be accessible only to JT and let the tissue be derived from a malignant kidney. Then it would

have the mask

| | | |
|---|---|---|
| Collaborator | 2 | code for JT |
| Project | 0 | no project specified |
| Organ | 3 | Kidney |
| Normality | 1 | Malignant |

Then, CMASK would take “01000301”. MASK would have the value (01 00 03 01) base N. In another embodiment of the present invention, the clinical database is built on an Oracle 8i database server.

The tissue repository information management system is the information system that manages the bio-repository. In addition, to being an inventory system, this system provides data entry tools for pathology and clinical records of bio-samples. The tissue repository information management system preferably runs on a MicroSoft Access back-end database. A server side script preferably exports the data from the Access database files as ASCII text files. These files are then transferred, preferably by means of ftp, to the pre-staging area and then loaded on the staging database for clinical data. During loading, the integrity of clinical data is checked through a list of rules, such as donor age should be in the range of [1, 99], weight should be expressed in metric system units, etc.

Only a subset of the data from the tissue repository information management system is needed for the clinical database, and the loading protocol preferably selects only those that are appropriate. After all the checks return successfully, new data is migrated to the relational database.

The schema for the tissue repository information management system can be preferably divided into three data units: (1) tissue details; (2) donor attributes; and (3) controlled vocabularies.

Sample detail attributes are organized in the BIOSAMPLE and FRAGMENT tables. BIOSAMPLE holds tissue specific attributes such as SITE (accrual site), SOURCE (accrual source), ORGAN_NAME. HISTOLOGY, PATIENT DIAGNOSIS, and PATHOLOGY DIAGNOSIS. BIOSAMPLE captures information about physical bio-sample entity.

A tissue FRAGMENT is a physical fragment of a bio-sample. These fragments are run through the experiments and are assigned a unique GENOMICS number. The FRAGMENT table also holds other attributes of the fragment such as WEIGHT_ACTUAL (actual weight in metric units i.e., kg), WEIGHT_ESIMATED. Organ name and histology fields relate to a standardized terminology, such as found in SNOMED and take values from a controlled vocabulary (CV). Similarly, the diagnosis field relates to SNOMED and have an associated CV.

A main table is DONOR. It has human donor attributes that that span various domains: general attributes such as HEIGHT, WEIGHT, RACE, DATE_OF_BITH; deceased fields such as DEATH_CAUSE, DEATH_AGE; sparse data fields such as exercise habits, diet profile, sleeping and smoking habits, alcohol and any recreation drug habits.

The DONOR fact table is preferably linked to five other detail tables: HISTORY_FAMILY—donor family diagnosis; HISTORY_MEDICAL—patient medical history; HISTORY_SURGICAL—patient surgical history and anesthesia (in HISTORY_SURGICAL_ANESTHESIA); HISTORY_MEDICATION—patient medications history; and HISTORY_LAB_TEST—patient lab test history.

An attribute that links the clinical database to other components is the genomics identification number. All fragments run through the chip gene expression get a unique genomics identification number. These identifiers are assigned during sample preparation and form a part of the experiment names. The genomics identification number is also stored in the fragment table. The ABS_GENE_EXPR_RESULT, ANALYSIS, EXPERIMENT, GLGC_EXPERIMENT tables in the gene expression data schema have the BIOSAMPLE ID field that contains the sample id in the clinical database for experiments run through the corresponding samples. This process is done as a part of the clinical data loading protocol, a stored procedure updates the above tables on the production database to do the job. The same stored procedure script is also run when new experiments are published to the production warehouse.

The relational database of the present invention preferably utilizes a three-layer archiving system. The three layers are: (1) an on-line network disk file system; (2) near-line storage; and (3) off-line DLT tape backups The on-line network disk file system is based on a network disk system (Network Appliance F720). The network file system is also visible to the NT network. The disk space is organized into two partitions: one for archiving and one for building data distributions. A complete set of information for each sample in a file system accessible from both UNIX and Windows is maintained. The information is organized by genomics identification number and can be further broken down by experiment name. By storing the information in this directory structure, it is easier to build distribution sets based on filtering requirements. The near-line storage is based the HP Superstore magneto-optical jukebox and serves as the backup device of all data files generated by production and is also the backup of the on-line archive.

Off-line DLT tape backups are used to backup the pre-staging directories, the database servers and the on-line archive.

Another aspect of the present invention is modifying the database to utilize new chipsets. It will be appreciated that periodically new gene chips for analyzing gene expression in tissues from various species will be available; these are preferably grouped in chipsets of 3 to 5 chips. Preferred gene sets include the Hu42K set for humans, the Mu11K set for mice, and the RG_U34 set for rats. Another preferred gene set is the Affymetrix HG_U95 chipset, also known as the 60K set (because the five chips in it represent about 60,000 gene fragments).

Although most of the gene fragments represented in the two human gene sets have counterparts, the oligonucleotides used to probe each fragment may differ between the two sets. In such circumstances, cross-chipset analysis is not available; gene sets may not contain a mixture of gene fragments from different chipsets. Further, sample queries are preferably restricted by chipset as well as by species; all samples in the sample set must have experiments from chips of the chipset that was selected when the query was run. The chipset used to qualify the sample query is saved as an attribute of the sample set.

Additionally, analyses are restricted by the chipset associated with the sample sets that are input for the analysis; when multiple sample sets are input, the sample sets must have all the same chipset attributes. The gene sets that are generated by the analysis will be filtered to contain only gene fragments for this chipset. Another aspect of the present invention is normalization of the data. Normalization makes the expression values reported from different gene chip experiments comparable to one another, so that if two different samples yield the same expression value for a gene fragment, there is reasonable confidence that the concentrations of mRNA transcripts for the fragment are the same in the two samples. Because of variations in the manufacturing process for the chips, as well as other factors, the unnormalized intensity values vary widely from one chip experiment to another for fragments with the same RNA concentration.

There are a number of preferred methods for adjusting for this variation. Preferably, the present invention supports three methods: scaling, normalization, and standard curve normalization. In scaling, average differential intensity values (or “AveDiffs”) are generated as a result of this normalization process. The normalized values are computed by multiplying the unnormalized values by a scale factor. The scale factor is the same for all values in an experiment, and is calculated as follows:

1. Take all the unnormalized AveDiff values in the experiment. Throw away the largest 2% and the smallest 2% of the values. That is, if the experiment yields 10,000 expression values, order the values and throw away the smallest 200 and the largest 200.

2. Compute the “trimmed mean,” equal to the mean of the remaining values.

3. Compute the scale factor SF =100/(trimmed mean).

Another normalization method is based on the observation that the expression intensity values from a single chip experiment have different distributions, depending on whether small or large expression values are considered. Small values, which are assumed to be mostly noise, are approximately normally distributed with mean zero, while larger values roughly obey a log-normal distribution; that is, their logarithms are normally distributed with some nonzero mean. While scaling applies the same scale factor to all expression values in an experiment, normalization computes separate scale factors for “non-expressors” (small values) and “expressors” (large ones). The inputs to the algorithm are the scaling AveDiff values, which are already scaled to set the trimmed mean equal to 100. The algorithm computes the standard deviation SD noise of the negative values, which are assumed to come from non-expressors. It then multiplies all negative values, as well as all positive values less than 2.0* SD noise, by a scale factor proportional to 1/SD noise. Values greater than 2.0* SD noise are assumed to come from expressors. For these values, the standard deviation SD log(signal) of the logarithms is calculated. The logarithms are then multiplied by a scale factor proportional to 1/SD log(signal) and exponentiated. The resulting values are then multiplied by another scale factor, chosen so there will be no discontinuity in the normalized values from unscaled values on either side of 2.0* SD noise.

A third normalization method is termed “standard curve norrnalization” or sometimes “spike-in normalization.” This normalization method relates the original expression intensity values from the chip experiments to actual mRNA concentrations for each gene expressed in the sample. In order to do this, known concentrations of particular gene fragments must be “spiked in” to the sample RNA mixture before hybridizing it to the chips. (Bacterial genes are used for the spike-ins, so there will not be any additional RNA contribution from the sample donor.) The chip experiment yields intensity measurements for the spike-in gene fragments. Ideally, the intensities will increase linearly with concentration; therefore, if intensity is plotted vs. concentration, it should be possible to draw a straight line through the origin connecting the data points, and use its slope to infer the mRNA concentrations for the other gene fragments on the chip. In reality there are noise and non-linear effects which distort this relationship; but one can still draw a straight line through the origin that is the best fit to the data points. The straight line is known as the “standard curve.” To perform standard curve normalization, the runtime engine (RTE) loader fits a standard curve for each chip experiment for which spike-in data is available, and divides the intensity measurement for each gene fragment by the slope of the standard curve to obtain a concentration value. (Negative values and values below a certain sensitivity cutoff are mapped differently; this mapping is described in a separate document.) The concentration value (in picomoles) is reported as the expression value, rather than the intensity.

Because only a portion of the samples may have spike-ins, the RTE will not generate concentration values for samples that do not have spike-ins. Therefore, when running an analysis tool such as Fold Change, if the standard curve normalization is selected, the present invention checks to see if all the samples in the input sample sets have sufficient spike-ins. If not, the database will issue a warning that certain samples cannot be used in the analysis and will terminate the computation. Additionally, concentration values fall in a different range (typically smaller) than intensity values, thus, it is necessary to use a smaller threshold when filtering the standard curve normalized data.

Another preferred embodiment of the present invention is a configuration of the database in combination with gene expression data obtained from restriction enzyme analysis of differentially expressed sequences (“READS”). Certain samples from toxicology experiments are processed using both platforms. The chip data are stored in the gene expression database. The READS data are stored in a separate database, known as ToxREADS. In a preferred embodiment of the present invention, links are created from certain data values in the database of the present invention to related ToxREADS data.

Most toxicology experiments are performed within the context of studies, in which groups of experimental animals or cell cultures are subjected to various treatments, and samples are collected from them at different time points post-treatment. For example, a study may examine the effect of two different doses of a toxin on rat livers at three different time points, compared to livers from saline-injected rats at the same time points. In order to improve the quality of the data, replicate experiments are performed; that is, several animals are treated with the same dose and sampled at the same time point. Each group of samples from replicate experiments is known as a study group. The Sample Set query tool allows you to search for samples belonging to a study and group them by study group.

READS data are derived from electrophoresis gels in which processed mRNA fragments from samples in different study groups are run on different lanes of the gel and separated by fragment length. Differentially expressed fragments, represented by bands that are darker in some lanes of the gel than others, are cored, sequenced, and matched to known genes if possible. As discussed above, data for these fragments, such as a measure of the intensity of the band, are stored in the ToxREADS database. Some of these gene fragments found in READS gels (known as READS fragments) may also be represented on one or more gene chips. In this case, expression data may be available from both platforms. Preferably, a link is created from the gene expression database data display to a ToxExpress report, so that the READS data and chip data may be viewed side by side.

It is important to note that expression data for READS fragments are only meaningful within the context of a particular study; thus, a user must choose the study he or she is interested in. When the user selects to add a ToxREADS link, the tool preferably displays a dialog box listing the available studies. The user then selects one or more studies from this list and clicks the Add button in the dialog; the results table will then display an additional ToxREADS link column for each study selected. The ToxREADS link column displays an arrow icon for each gene fragment in the query results that is associated with a READS fragment in the study for that column. When the user clicks on this icon, the gene expression database directs the user's Web browser to navigate to the report page for the corresponding READS fragment in the associated study. Each lane of a READS gel (and therefore, each band corresponding to a READS fragment) may be derived from several individual samples that are pooled together. Typically, the samples in each study group are pooled together, so that there is one READS sample per study group; further, the control samples for different time points (which are stored in the gene expression sample database in separate study groups) are pooled together into one READS control sample.

To make it easier for a user to relate individual samples to pooled READS samples, ToxExpress users are preferably provided with a collection of predefined sample sets. These are organized under subfolders for each ToxExpress study; each sample set contains the samples corresponding to a pooled READS sample. When the user clicks on a ToxREADS link in gene expression database, a report is preferably displayed showing information about the READS fragment associated with a selected gene fragment within a particular study. The rows of the table may correspond to different pooled READS samples in the study; the rightmost columns may show the expression intensity value from each READS experiment, and the mean expression values (with both scaling and normalization) from the corresponding chip experiments. Some of the fields in the table (e.g., READS Fragment) may have arrow icons associated with them. These can act as links to detail reports. For example, when the user clicks on the icon next to a READS Fragment name, the user's Web browser navigates to the detail report for that READS fragment.

Each READS Fragment detail report preferably contains a link to a chromatogram trace file. In order to view this file, the Web browser must be configured to launch a program capable of reading and displaying the file. Another aspect of the present invention is a gene signature analysis. A gene signature analysis of a sample set extracts two sets of gene fragments from all of the gene fragments represented in the sample set's chipset: those that are consistently expressed within the sample set, and those that are consistently not expressed. In order to perform the gene signature analysis, it is necessary to quantify the “consistency” of expression as two threshold percentages, one for the “present” set, the other for the “absent” set. Consistency of expression is a measure of how much a gene (fragment) is expressed, or not expressed, in a sample set. For example, if there are 5 samples in the sample set, and the user sets the present and absent threshold percentages to 80% and 80%, respectively, then the gene signature analysis computes one set of genes that are present in at least 4 out of 5 samples, and another set which are absent in at least 4 of 5 samples. There are a variety of ways in which the result of the gene signature analysis can be displayed. After the analysis is complete, the results are preferably displayed in the summary tab of the gene signature analysis window. This window preferably presents a panel displaying the number of gene fragments in the present gene set; a panel displaying the number of gene fragments in the absent gene set; and the name of the sample set and the number of samples it contains. Default summary columns preferably include GenomicslD, Experiment(s), Total Present Calls, Total Absent Calls, Total Unknown Calls, Present Calls (Present Gene Set), Unknown Calls (Present Gene Set), Absent Calls (Absent Gene Set), and Unknown Calls (Absent Gene Set). At the bottom of the window, the Gene Signature History is preferably displayed. This presents information about the thresholds used to compute the analysis, the date and time the analysis was performed, and the version of the Runtime Engine (RTE) used for the analysis.

In another embodiment of the present invention, the display of the gene signature analysis permits display of details regarding the gene signature analysis. The options preferably include Sample Detail, Attributes, Experiments, Sample, Donor, and Display Options. In another preferred embodiment, it is possible to export the summary into an Excel worksheet, export the summary into a Web browser, or print the summary.

In viewing the gene signature curves, there are preferably two display options: Number of Fragments vs. Number of Samples and the Number of Fragments vs. Threshold Percentage. The Number of Fragments vs. Number of Samples option displays a pair of gene signature curves, one for the present gene set and one for the absent gene set. This display is designed to give the user a visual sense of whether the sample set is large enough to generate a valid gene signature. The Number of Fragments vs. Threshold Percentage option displays the counts of the present and absent genes as a function of the threshold percentage. For example, if both thresholds were set to 90%, which means that qualified fragments should be present or absent in 31 out of 34 samples, the number of fragments in the present and absent set would be approximately 4,000 and 17,000 respectively. If the thresholds were set at 75% (less stringent) the sets grow to 7,944 and 24,155 respectively. Detailed information about the gene fragments results are preferably displayed in the Gene Set Results. Fr example, to view a list of gene fragments in the present or absent gene set, a Gene Set Results window preferably presents a drop-down box to select either a vertical or horizontal split view of the results, a tab that displays the Present Gene Set results, a tab that displays the Absent Gene Set results, the number of genes in the Present or Absent Gene set, depending on which tab is selected, a statement about the type of normalization used, and a table of gene results in both the Present or Absent Gene Set view.

In another preferred embodiment of the present invention, detailed information about selected gene fragments is displayed. The options preferably include Fragment Details, Attributes, Known Gene, Sample Details, Attributes, Experiments, Sample, Donor, and Sequence Cluster. Another aspect of the present invention is the ability to view gene fragments in a sequence cluster. The sequence cluster option presents a view of a gene fragment in the context of the Unigene cluster it is classified under. It is also possible to view a table with the expression values of all gene fragments in the same Unigene cluster over the corresponding sample or sample set.

The present invention also permits the display of data regarding specific fragments in combination with user-selected gene attributes. These attributes preferably include gene signature stats (present frequency, mean, median, standard deviation, expression and call values (one row per gene, where the present/absent calls and quantitative expression values for the fragment across all samples in the sample set is displayed), and expression and call values (one row per gene per sample, where one row per fragment per sample including the actual present/absent call and the quantitative expression value for the fragment). Another aspect of the present invention is a Pathway Viewer which presents a pathway display where expression values are overlaid on known pathways. The proteins or enzymes that are encoded by genes are highlighted with colored bands. Colors can represent the expression levels of the gene fragments, with more intense colors for extreme expression values (negative and positive). Clicking on a colored band can open a detail window that displays additional information about the expression levels of the gene fragments encoding the enzyme or protein. When a detail window is open and a different gene fragment in the table is selected, a new set of proteins or enzymes is preferably highlighted (unless the fragment maps to the same set of nodes). If the fragment maps to more than one protein or enzyme, the application preferably selects one at random, scrolls it into view if necessary, and updates the detail window display. It is also possible to obtain a full view of the pathway or to zoom into a particular area of a pathway. When a gene fragment is selected in the pathway table, all the nodes in the pathway that the fragment maps to are preferably “highlighted.”

The display of the pathway is provided in several formats, preferably including median values for the sample set (the median expression values are displayed for each fragment in the selected gene set that overlaps the pathway, over all samples in the input sample set), mean values for the sample set (the mean expression levels are displayed for each fragment in the selected gene set that overlaps the pathway, over all samples in the input sample set), and raw expression values (the raw expression levels will be displayed for each fragment in the selected gene set that overlaps the pathway, over all samples in the input sample set).

Another aspect of the present invention is a chromosome viewer which presents a display that renders expression values over a chromosome map. The chromosome diagram preferably displays a statement about the number of markers, and the number of matches displayed; that is, the total number of fragments on the chromosome, and the number from the current gene set; a statement about the display option; a table containing results data; a panel displaying the chromosome image, along with a vertical axis that displays the expression values. In a preferred embodiment, to determine where a gene fragment maps on the chromosome, the gene fragment is selected from the table and in the chromosome diagram, the corresponding gene fragments will be indicated. There are preferred display options for the chromosome viewer. These include median values for sample set; mean values for sample set; raw expression values for samples; and present/absent call values for the samples.

Another aspect of the invention is a gene mask option which provides a means of filtering the gene set, allowing for either intersecting gene sets to reveal shared genes, or to display differences between gene sets. For computing the gene signature analysis, fragments that have “marginal” calls for a particular sample are treated the same as “absent” fragments. Fragments that have “unknown” calls are ignored in the gene signature computation. If, for a particular fragment, p, m, and a are the numbers of samples for which the fragment was present, marginal, and absent, respectively, then the fractions p/(p+m+a) and (m+a)/(p+m+a) are computed; these fractions are compared against the present and absent threshold percentages to determine if the fragment belongs to either of the gene signature gene sets. For example, suppose the gene expression data warehouse contained the present/absent/marginal/unknown call values shown in the table below, for the sample set S={s1, s2, s3, s4} and the genes {g1, g2, g3, g4, g5, g6, g7, g8, g9}. (In reality there would be data for thousands of genes, but only nine genes are shown for illustration.) At the bottom of the column for each gene are shown the percentages computed from the numbers of present, absent, and marginal calls for each gene across sample set S.

| | gi | g2 | g3 | g4 | g5 | g6 | G7 | g8 | g9 |
|---|---|---|---|---|---|---|---|---|---|
| s1 | P | P | P | P | A | A | A | A | A |
| s2 | P | P | P | P | A | A | M | P | A |
| s3 | P | P | P | P | M | U | M | U | A |
| s4 | P | P | M | U | M | U | P | U | A |
| P % | 100 | 100 | 75 | 100 | 0 | 0 | 25 | 50 | 0 |
| A % | 0 | 0 | 25 | 0 | 100 | 100 | 75 | 50 | 100 |

Suppose that the present and absent threshold percentages were both set to 75%. Then for this sample set, the gene signature operation returns a “present Gene Set” containing genes {g1, g2, g3, g4}, and an “absent Gene Set” containing {g5, g6, g7, g9}. The gene signature analysis also computes the mean, median, and standard deviation for each gene in the present and absent sets. The user can select any or all of these values to be displayed in the gene signature results.

The curves for the gene signature are computed by computing the present gene counts for each sample in the sample set; ordering the samples by present gene count in ascending order; initializing P to the set of present genes in the first sample (the height of the first point in the curve is the number of genes in P); intersecting P with the set of present genes in the second sample, and repeating for each sample in the sample set. The heights of the successive points in the curve are the number of genes in P after each intersection step. The X axis component of each point is the index of the corresponding sample in the sorted sample set. This analysis is also performed for the absent genes, and the intersection set counts are plotted on separate graphs. The method used to produce the gene signature present and absent gene sets is not the same as the algorithm used to compute the gene signature curve. The gene signature computation utilizes a threshold percentage to obtain the Present/Absent Gene Sets, while the curve computation does not.

Furthermore, U (unknown) and N (no expression data—that is, samples with missing

chips) calls play a crucial role in producing discrepancies between the gene signature and

the Gene Signature Curve. For example, consider the call value matrix below where the

Si are samples and Gi are genes.

| | G1 | G2 | G3 | G4 |
|---|---|---|---|---|
| S1 | P | P | P | U |
| S2 | P | P | U | P |
| S3 | P | U | P | P |
| S4 | U | P | P | P |

A gene signature computation to get the Present Gene Set with 100% threshold would yield the following Gene Set {G1, G2, G3, G4}, with a count of four genes. The calculation algorithm does correct for partial chip sets and missing data by including only the samples for which there are expression data. Thus, all four genes are included in the Present Gene Set, even though each of them is only called present in three out of the four samples. A gene signature curve, however, would yield the following data for the Present Gene Set.

$$( Number of Samples \cdot Number of Genes ) = \{ ( 1 , 3 ) , ( 2.2 ) , ( 3 , 1 ) , ( 4 , 0 ) \}$$

$$\begin{matrix}Number of Genes & \begin{matrix}\begin{matrix}\begin{matrix}4 \text{-} \\ 3 \text{-}\end{matrix} \\ 2 \text{-}\end{matrix} \\ 1 \text{-}\end{matrix} \vert \\ & \overline{1 \; 2 \; 3 Number of Samples}\end{matrix}$$

In the present invention, the “Number of Genes” values equal to zero are not plotted. Thus, the maximum number of samples shown on the x-axis may differ from the number of samples in the sample set, and may even differ between the present and absent gene signature curves. The algorithm first orders the samples by the present count in ascending order, then initializes P to the set of present genes in the first sample. The height of the first bar in the curve is the number of genes in P. P is then intersected with the set of present genes in the second sample, and the number of genes remaining in P is shown as the height of the second bar in the curve. This process is repeated for each sample in the sample set. The U (unknown) and N (no data for sample) calls play a crucial role in producing these “disparities.” This example shows how the seeming disparities are produced by these two algorithms on the same data. Hence, one can obtain values where the last element in the histogram chart is not the same as the size of the gene set, as well as having the x-axis not equal to the size of the sample set.

Another aspect of the present invention is a gene signature differential analysis which compares the results of two gene signatures created using the gene expression database of the present invention. Using these two gene signatures, the analysis computes four new sets of gene fragments. A gene signature differential analysis compares two gene signatures (which must have been previously computed and saved). The analysis derives four new sets of gene fragments: those that are in both the first gene signature's present gene set and the second's absent gene set; those that are in both the first gene signature's absent gene set and the second's present gene set; those that are in both present gene sets; and those that are in both absent gene sets.

After obtaining the gene signature differential analysis, the results can be presented in a number of preferred formats, including a summary view, a gene set results view, a pathways view, and a chromosome map view. Preferably the summary view contains the following information: the names of the two input gene signatures, when they were last modified, the size of the sample sets used, the thresholds used to compute the gene signatures, the sizes of their present and absent gene sets, a table summarizing the number of gene fragments in the four intersection sets: Present only in <1st Gene Signature>, Present only in <2nd Gene Signature>, Present in Both (gene signatures), and Absent in Both (gene signatures), a history panel that records the date and time of the analysis and the version of the runtime engine used. The gene signature differential computes four new sets of fragments using the present and absent gene sets for two gene signatures. This is accomplished with the following sets: a set containing the fragments that are in the first gene signature's present set and the second's absent set; a set containing the fragments that are in the first gene signature's absent set and the second's present set; a set containing the fragments that are in both present sets; and a set containing the fragments that are in both absent sets.

Another aspect of the present invention is a Fold Change Analysis which compares the mean expression levels of each gene fragment in a chipset between a control sample set and an experimental sample set to compute a fold change ratio. The Fold Change Analysis quantifies the change in expression for differentially expressed genes between pairs of sample sets. After computing the fold changes for each fragment, the fragments are classified by fold change value.

The results of the fold change analysis are preferably displayed as a summary of the number of genes in each fold change bracket and the direction of the fold changes between the control and experimental set(s). preferably, such a summary displays a list of all of the control sample sets and the number of samples in each; a list of all of the experimental samples and the number of samples they contain; a check box which the user may select to include in the gene counts fragments that were absent in both the experimental and control sample sets; a table listing the number of gene fragments with fold changes in the following ranges: ●greater than 100●, between 10 and 100●, between 5 and 10●, between 4 and 5 ●, between 3 and 4●, between 2 and 3●, between 1 and 2●, and with no change.

The numbers are preferably broken down in the following manner: the number of fold changes “up” in the experimental versus the control set; the number of fold changes “down” in the experimental versus the control set; and the total of all changes in the experimental versus control set.

To obtain more specific data about the Fold Change Analysis results, the present invention preferably provides four different views of the results: filtering gene fragments, viewing gene fragments, viewing pathways, and viewing chromosome maps.

The Filter Gene Fragments view allows for filtering the reported genes using a previously saved gene set. The user selects the gene set to use as a filter; only genes contained in the filter will be displayed.

The Gene Fragments view preferably presents a drop-down box in which to select either the vertical or horizontal split view; a statement of the number of gene fragments displayed; and a table of gene results.

The Pathway View presents a pathway display where expression values are overlaid on known pathways.

The Chromosome View presents a display that renders expression values over a chromosome map.

A fold change analysis operates on quantitative expression values. It computes, for each of a set of selected gene fragments, the ratio of the geometric means of the expression intensities in a control sample set and an experimental sample set. The fold change is equal to this ratio. If the ratio is less than one, and the user has elected to display fold changes with magnitudes and directions, then the fold change magnitude is the reciprocal of the ratio, with a “down” direction. Multiple fold change comparisons may be run in parallel, between different experimental sample sets and matched control sample sets. The analysis categorizes gene fragments by the fold change of their mean expression values between each pair of sample sets, and reports detailed expression information for those fragments whose fold changes fall within a user-specified range, or for fragments in a user-specified gene set. Confidence limits and p-values are also calculated when possible. The algorithm is based on a two-sided Welch modified two-sample t-test. It assumes that the logarithms of the expression intensities for each sample set are normally distributed (which is a fairly good match to our data), and that the variance of each control sample set may differ from the variance of the experimental set it is being compared to. Note that the p-values are not corrected for multiple comparisons. The null hypothesis used for the t-test is that the population means for the logs of the expression values are the same in the two sample sets. The alternative hypothesis is that the means are different. The p-value reported is an estimate of the probability that a difference of means (and thus a fold change) as extreme as that observed could be obtained under the null hypothesis. Confidence limits on the fold change value are calculated according to the same set of assumptions. By default, 95% confidence limits are computed; a different confidence level can be specified by the user. The upper and lower 95% confidence limits reported are the estimated bounds of the interval for which, under the above assumptions, there is a 95% probability that the actual ratio of population means falls within the interval. Both sample sets must have more than one sample. If one or both of the sample sets has only one member, then confidence limits and p-values cannot be calculated, though a fold change is still reportable using the algorithm described below. Fold change is calculated on a per fragment basis: that is, the fold change algorithm is applied to each fragment separately. Users have the option to choose Gene Logic normalized, standard curve normalized, or Affymetrix normalized expression values for the analysis, but the same normalization must be used across all samples and genes. A floor is applied to the expression values with normalization or scaling; the floor value used is based on a noise parameter Q, which depends on the type of normalization chosen. For Gene Logic normalized expression values (“GL expression”), each chip has a standardized noise level Q equal to 10. More precisely, the distribution of the noise on each chip can be estimated as part of the normalization, and the expression values recalculated so that the standard deviation of GL expression values near 0 is equal to 10.

For scaling expression values, the analysis uses the actual noise value Q RawQ*SF calculated for each chip experiment by the Affymetrix software and stored in the GXDB database. The user also has the option to compute the fold change using only samples for each gene for which the gene is called present. When this option is selected, the numbers of samples nₓ and n_{y} for each sample set will vary for different genes, and it may not be possible to compute p-values and confidence limits for every gene. The inputs to the algorithm are two sample sets, X and Y, and one gene set; along with the user-specified confidence level CL (between 0 and 100%, defaulting to 95%).

The fold change algorithm is as follows. For sample set X and a gene fragment f in the gene set, do the following:

1. First apply a floor value to the expression data. Let e_{fi} be the normalized expression value for fragment f in sample i. If normalization is used, set e_{fi} to max(e_{fi}, 20). If scaling is used, set e_{fi} to max(e_{fi}, 2*SF_{fi}*RawQ_{fi}) where RawQ_{fi} and SF_{fi} are the RawQ and scale factor parameters from the chip experiment on the chip containing fragment f, for sample i. If the resulting e_{fi}<20, set e_{fi} to 20. If standard curve normalization is used, e_{fi}, is left alone and no floor value is applied.

2. Given expression levels {e_{fi}·i=1, 2, . . . , nₓ} across nₓ samples in sample set X, calculate the logs: xᵢ=1n(e_{fi}).

3. Calculate the mean(x), i.e., mean(x)=(sum over i of xᵢ)/nₓ.

4. Calculate the variance(x), i.e., var(x)=(sum over i of(xᵢ−mean(x))2)/(nₓ−1).

5. Repeat steps 1-4 for sample set Y.

6. Calculate a t statistic: t=(mean(x)−mean(y))/s

where s=sqrt(var(x)/nₓ+var(y)/n_{y})

7. The computation of the p-value and confidence limits requires the cumulative T probability distribution function Pt(t, DF) and the inverse function tInverse(p,DF).

Compute the (non-integral) degrees of freedom parameter:

DF=1/(c²/(nₓ−1)+((1−c)²)/(n_{y-}1))

where c=var(x)/(nₓ*s²)

8. Calculate the p-value by: Pval=Prob(|T|>t)=2*(1−Pt(t,DF))

where Pt(t, DF) is the cumulative T distribution with DF degrees of freedom and t is the statistic specified above.

9. Compute the fold change ratio FC and upper and lower confidence limits.

Given the user specified confidence level CL, compute: TI=s* tInverse((100+CL)/200, DF). The fold change and confidence limits are then calculated using:

m=mean(x)−mean(y) FC=exp(m)

Lower confidence limit=exp(m−TI)

Upper confidence limit=exp(m+TI)

The fold change direction is reported as “up” if FC>1 and “down” if FC <1; the fold change magnitude is FC if FC>1 and 1/FC if FC <1. After computing the fold

changes for each fragment between the control and experiment sample sets, the fragments are classified by fold change value, and a summary report is produced showing the counts of fragments with fold changes within certain ranges. Typically the user is interested in all gene fragments that have fold change magnitudes greater than a certain value.

Fragments for which all samples in both sample sets return an absent call may be included in or excluded from the counts. Absent Gene Filtering Given control and experiment sample sets and a gene G, the fold change for G is computed as the ratio of the geometric means of the intensities for gene G over the two sample sets.

If the user selects to use only samples where gene is present, then the intensities for the samples where G is called absent are excluded from the geometric mean calculation; otherwise all intensities are included. In both cases, a floor value is applied to the intensities, depending on the normalization selected. If normalization is used, the floor value is 20 (that is, all intensities less than 20 are replaced with 20 before calculating the geometric means). If scaling is selected, the floor value applied to the intensities from a particular chip experiment is twice the Q value computed for that experiment (that is, a different floor value is used for each sample/chip pair).

Confidence Level Confidence limits are calculated using a two-sided Welch modified t-test on the difference of the means of the logs of the intensities. The Welch form of the t-test is used because variances are generally unequal between the two groups of samples being compared. The logs of the intensities are assumed to come from a normal distribution, which matches our observations for the nonnegative values. The confidence bounds are no longer symmetric about the fold change estimate on an additive scale; however, they are symmetric about the fold change estimate on a multiplicative scale, which is the appropriate type of scale for ratios (such as fold changes).

Another aspect of the present invention is an Electronic Northern Analysis (E Northern) which takes a user-defined gene set and one or more sample sets as input and reports the range of expression levels for each gene fragment in the gene set across each sample set, for all of the samples with user-specified present/absent calls.

The range of expression values for a gene in an E Northern analysis is preferably reported as a pair of user-selected percentiles over the values for the samples in each sample set. By default, the values at the 25th and 75th percentiles over each sample set are shown. The user may select different percentiles. For example, the user may choose to view the 0th percentile (the minimum expression value) and the 100th percentile (the maximum) for each sample set. In addition to the user-specified percentiles, the median expression value (the 50th percentile) is preferably reported.

The electronic northern analysis is computed using one or more sample sets and a gene set. The gene set can be either a gene set that was created and saved previously or the resulting gene set of a gene signature differential.

The electronic northern analysis preferred display of the results includes a drop-down list in which to choose either a vertical or horizontal split view; the number of Affymetrix fragments; the number of rows; the upper and lower percentiles used; the normalization used; and the call types (present, absent or marginal) used to compute the percentiles.

In another preferred embodiment of the present invention, the electronic northern analysis will preferably display detailed information about selected gene fragment, including fragment; attributes; known gene; sample details; experiments; sample; donor; sequence cluster; and E Northern plot.

The E Northern Plot displays a visual representation of Electronic Northern results and expression values for the selected Affymetrix fragment. The top part of the E Northern plot view displays selected attributes of the Affymetrix fragment. The plot shows tick marks or circles corresponding to the expression values for individual samples, overlaid with a translucent box plot in which the ends of the box represent the user-specified percentile values. The plot also displays multiple rows for a gene, one per input sample set; these are paired with bar graphs showing the percentage of samples in each sample set in which the gene is called present. Vertical bars are displayed at the median and at the median plus or minus 1.5 times the interquartile range. The X axis of the plot shows graduated markers.

An Electronic Northern Analysis (or E Northern) takes as input a user-defined gene set and one or more sample sets, and reports the range of expression levels for each Affymetrix gene fragment in the gene set across each sample set, over all the samples with user specified present/absent call values. The range is reported using percentile values, with the upper and lower percentile levels U and L specified by the user. If the user chooses U to be 100 and L to be 0, the analysis reports the maximum and minimum expression values over the selected samples. If the user chooses U 75 and L 25, the upper and lower quartile values are reported. The median value is reported as well.

The E Northern is computed as follows for each sample set:

1. The user's selection in the E Northern Options dialog is used to determine how samples with Absent and Marginal calls will be used in the computations. If “Include Present calls only in computation” is selected, only samples with Present calls are used in the percentile and present score computations; Marginal calls are treated the same as Absent calls and are included in the absent score. If “Include Present and Marginal calls in computation” is selected, samples with either Present or Marginal calls are included in the percentile and present score computations. If “Include Present, Marginal, and Absent calls in computation” is selected, samples with Present, Marginal or Absent calls are used to compute the percentiles, and Marginal calls are included in the present score.

2. For each gene fragment in the user-specified gene set, present and absent scores are computed by counting the numbers of Present and Absent calls for the samples in the given sample set, and dividing each count by the total number of samples that have expression data for the gene fragment. Samples with Unknown and Null calls are omitted and are not included in the total count of samples. The result is reported as a fraction in the tabular display (e.g., 17/22) and as a percentage in the E Northern plot.

3. For each gene fragment, the percentile and median values are computed over

the samples with user-selected call values. The expression values for these samples are first sorted in ascending order. This generates a rank order R for each expression value, R=1 . . . N; where N is the number of selected samples. Define X_{R} as the expression value with rank order R.

4. Three percentile values are computed: the 50th percentile (i.e., the median), and

the two user specified percentiles L and U. The Pth percentile of a set of values is the value X such that P percent of the values in the set are less than X.

5. Let M=1 +((P/100)*(N−1)).

6. If M is an integer, the Pth percentile is X M, the expression value with rank order M.

7. If M is not an integer, the Pth percentile is obtained by interpolating between the values X M and X_{M+1}. Let F be the fractional part of M. Then the Pth percentile is computed as X M+F*(X_{M+1}−Xₘ)

8. The above calculation is performed for P=L, P=50, and P=U.

The present invention provides a system and method of analyzing gene expression, gene annotation, and sample information in a relational format supporting efficient exploration and analysis, comprising: providing a data warehouse which comprises a gene expression database for storing quantitative gene expression measurements for tissues and cell lines screened using various assays; a clinical database for storing information on bio-samples and donors; and a fragment index for biological properties for DNA fragments; receiving a query regarding gene expression of one or more DNA fragments; determining the level of gene expression of the one or more DNA fragments; correlating the level of gene expression with the clinical database and the fragment index; and displaying the results of said correlation.

An aspect of the present invention is a series of databases that contain gene expression data for tens of thousands of genes, measured over thousands of samples. The present invention provides tools for a user to extract subsets of clinical and genetic data, perform analyses, and display the results.

It will be appreciated that an aspect of the invention is the installation of the application. There are several aspects to installing the application, including system requirements, installation of the application; installation of the Java Runtime Environment; and downloading the installer.

With regard to system requirements, preferably the present invention requires a 500 MHz Pentium III processor running Windows NT 4.0 or later with at least 256 MB of RAM and virtual memory set to 256 MB; a color monitor with at least 1024×864 pixels and 256 colors (1152×864 pixels and 65536 colors are recommended); Netscape Navigator (version 4.7) or Internet Explorer (version 5.0 or later); a URL provided by the user for the invention's installation Web page; a workspace account; and a Java Runtime Environment (JRE), which may be downloaded from the invention's installation page.

In addition, other commercially software packages are preferably available to augment the present invention, including Spotfire Pro (version 4.0 or later); Spotfire Array Explorer; Microsoft Excel 2000; Eisen Cluster Tool; and GeneSpring; Partek Pro 2000.

To install the application of the present invention, a user preferably point his/her Web browser to the URL providing the home page of the present invention. The user can then select the download option, which opens the download and installation page of the present invention. Among other things, this page provides instructions for completing the two steps for installing the application of the present invention: installing the Java Runtime Environment and downloading the installer of the present invention.

In a preferred embodiment of the present invention, the application utilizes user profile information including full name, email, facsimile number, telephone number, and other contact information.

Over time, a user of the application of the present invention will develop a large number of sample sets, gene sets, and analysis results. The application of the present invention preferably incorporates a workspace which serves as a centralized repository for these data objects, organized into user-defined project folders. Access to the workspace is preferably controlled through user names, user group affiliations, and passwords. User-defined data objects are by default private to the user; however, during the save process, the user preferably has the option of making data objects accessible to other users.

The workspace window of the application of the present invention preferably contains the following components: a menu bar; quick access icons; a main window; and a status bar.

The menu bar preferably contains the following menu items: a File tab; an edit tab; a Queries tab; an analyses tab; a view tab; a Window tab; and a Help tab.

Under the File tab are preferably found several tabs, including an Open tab which opens a selected data object; a New Folder tab which creates a new project folder; a Properties tab which opens the Properties window; and an Exit tab which exits the application.

Under the Edit tab are preferably found several tabs, including a Cut tab which cuts the selected object; a Copy tab which copies the selected object; a Paste tab which pastes the last cut or copied object; a Delete tab which deletes the selected object; a Rename tab which enables the renaming of the selected object; and a Set Permissions tab which opens the Permissions window where access permissions can be set for the selected object.

Under the Queries tab are preferably found several tabs, including a Sample Set tab which displays a Sample Set window and a Gene Set tab which displays a Gene Query window.

Under the Analyses tab are preferably found several tabs, including a Gene Signature tab which displays a Gene Signature Analysis window; a Gene Signature Differential tab which displays a Gene Signature Differential Analysis window; a Fold Change Analysis tab which displays a Fold Change Analysis window; an ENorthern tab which displays an Electronic Northern window; an Expression Data Tool tab which displays an Expression Data Tool window; and a Contrast Analysis tab which displays a Contrast Analysis window.

Under the View tab are preferably found several tabs, including a Toolbar tab which toggles the toolbar on and off; a Status Bar tab which toggles the status bar on and off; and a Workspace tab which enables a user to select various options for viewing including View All Folders which shows accessible folders and data objects for all users; My Folder which shows only the user's folder and data objects; Sample Sets which shows only folders and Sample Sets; Gene Sets which shows only folders and Gene Sets. The View tab preferably includes a Sort Table by Name tab which sorts the data objects by name, a Sort Table by Class which sorts the data objects by object type, and a Sort Table by Date which sorts the data objects by the date they were last modified. Under the View tab is also preferably found a My Profile tab which opens the User Profile window where password and contact information can be updated. A ToolTip Customizer tab which opens the ToolTip Customizer window where settings for tooltip displays can be applied is also preferably found under the View tab. Under the View tab is also preferably found a Refresh Selected tab which refreshes the display of a selected folder's contents and a Refresh All tab which refreshes all of the folders.

Under the Windows tab are preferably found several tabs, including a Workspace tab which brings the workspace window to the foreground; an Arrange All tab which makes all open windows visible and arranges them on the desktop; a Minimize All tab which minimizes all but the workspace window; a Maximize All tab which maximizes all windows; and an <open windows>tab which lists the windows of the application that are currently open and allows one to select one of the items to bring that window to the foreground.

Under Help tab are preferably found several tabs, including a Help tab which accesses the Help system; a Home Page tab which launches a new browser window, if one is not already open, and points to the application's Home Page; an Error Log tab which displays the error log; and an About tab which displays information about the version of the application of the present invention.

In another preferred embodiment of the present invention, quick access icons are preferably provided including a Sample Set icon which displays a new Sample Set query window and is used to select criteria and query the clinical database for a set of tissue, cell culture, or cell line samples; a Gene Set icon which displays a new Gene Query window and is used to select criteria and query the Fragment Index database for a set of gene fragments; a Gene Signature icon which displays a new Gene Signature Analysis window and is used to identify which genes are present and which are absent in a given sample set; a Gene Signature Differential icon which displays a new Gene Signature Differential Analysis window and is used to compare the gene signature analyses of two given sample sets; a Fold Change icon which displays a new Fold Change Analysis window and is used to compute ratios of mean expression levels of genes between pairs of sample sets; an Electronic Northern icon which displays a new Electronic Northern Analysis window and is used to report and display graphically the range of expression levels for each gene fragment in a gene set(s) across one or more sample sets; an Expression Data Tool icon which displays a new Expression Data Tool window and is used to visualize expression data for the gene fragments in a gene set(s) across one or more sample sets; and a Contrast Analysis icon which displays a new Contrast Analysis window and is used to find genes that fit a pattern of expression.

Preferably the application of the present invention includes a Main Window consisting of two areas: a tree display showing the folders and objects in the workspace, with the user's folders on top, followed by the public folder, followed by the folders of other users, and a panel that shows detailed information about the objects in the currently selected folder, including their names, their class names (that is, the type of query or analysis), the chipsets used to create them, their owners, the date they were last modified, access permissions indicating which users can read (view) the object, and access permission indicating which users can write to (modify) the object.

Preferably the public folders of the application of the present invention include pre-defined gene and sample sets, including under Gene Sets By Chip—sets of all gene fragments for each chip type; Gene Sets By Chip Set—sets of all gene fragments for each chipset; Controls—all control gene fragments, grouped by chipset; Pathways—gene fragments for metabolic and signaling pathways, organized by chipset; and QC Controls—gene fragments used for RNA quality control, grouped by chipset. Under Sample Sets is preferably found Normal Mice—each sample set contains a particular strain of normal (that is, untreated) mice; Normal Rats—each sample set contains a particular strain of normal (that is, untreated) rats; and ToxExpress—contains sample sets for toxicology study groups and pooled READS samples.

In a preferred embodiment of the application of the present invention it is possible to view the properties of a data object: for example, the name of the object, the class of the object, the object path, the chipset used to create the object, a description of the object, and the access permissions for the object.

Tooltip information is preferably displayed throughout the application by holding the mouse cursor over certain features. If there is a tooltip associated with a feature, additional information about it is displayed in a textbox. Tooltips are especially helpful when viewing chromosome information. Preferably it is possible to customize the timing of the tooltip displays, or, in other words, to set the length of time the tooltip is displayed on the desktop.

In a preferred embodiment of the present invention, the user can create a sample set. A sample set is a group of biological samples within the application containing gene expression data. A user can define sample sets by specifying a combination of query criteria that are applied to the clinical data in the database. Upon completion of the query, the application of the present invention displays a list of samples satisfying the criteria.

The application of the present invention contains data from gene chip experiments on a large variety of tissue, cell culture, and cell line samples, from humans, mice and rats. Hundreds of attributes are maintained for the samples, including donor characteristics, medical history, laboratory tests, and so on. Some attributes are stored for all samples; certain other sets of attributes are only maintained for specific species and sample types. For example, alcohol usage attributes are not stored for animal tissue, cell culture, and cell line samples.

Gene chips are preferably grouped into sets of three to five chip types, each chipset containing probes for genes of a single species. Sample sets are constrained to only contain samples of a single species. In some cases, the expression database of the present invention contains data from more than one chipset for the same species. For this reason, sample sets are preferably subject to a further constraint: all samples in a sample set must have experiments in the database from a single chipset. The user must specify the chipset to be used to constrain the sample set by selecting it from the Chipset menu prior to running the query.

Preferably there are several types of samples, including tissue, primary cell culture, and cell line. It is possible for samples of different types to be mixed in a single sample set. However, in order to query against attributes that only apply to a specific sample type, the user must specify the type by selecting it from the Type menu before selecting any attributes.

For example, Affymetrix periodically releases new gene chips for analyzing gene expression in tissues from various species; these are grouped in chipsets of 3 to 5 chips. It is possible that the database of the present invention contains a mixture of data derived from multiple chipsets per species. Although most of the gene fragments represented in a set may have counterparts in other sets, the oligos used to probe each fragment differ between the two sets. This means that gene sets may not contain a mixture of gene fragments from different chipsets; that sample queries are restricted by chipset as well as by species; all samples in the sample set must have experiments from chips of the chipset that was selected when the query was run; that the chipset used to qualify the sample query will be saved as an attribute of the sample set; that analyses are restricted by the chipset associated with the sample sets that are input for the analysis; when multiple sample sets are input, sample sets must have all the same chipset attributes; and that the gene sets that are generated by the analysis will be filtered to contain only gene fragments for this chipset.

To access the Sample Set query window, from the Queries menu select Sample Set, or click on the Sample Set icon in the workspace window. A Sample Set query window opens on the desktop:

In a preferred embodiment of the present invention, the application provides for a sample set query. In general, the sample set query allows the user to select sets of samples with specific characteristics. For example, a sample set of tissues can be selected that indicate fibrosis of the liver. A series of steps are involved in specifying the search parameters. These include: selecting the appropriate subset of the database to search. In this case, the chipset will be specified as “H.sapiens (HG_U95),” and the sample type will be specified as “tissue;” selecting the first attribute on which the query will be based. In this case, the organ is “liver;” selecting the second attribute on which the query will be based. In this case, the sample pathology/morphology will be “fibrosis;” selecting laboratory test attributes; selecting search options; selecting “sort by” options; and performing the search.

It will be appreciated that the results can be viewed in a number of different formats. In one preferred format of the present invention, the results of the sample set query will automatically be displayed in a Results panel of the Sample Set window. This window presents the following information: a statement above the results indicating the parameters used in the search; a statement indicating the total number of samples found in the query, and the number currently selected; and a table of samples returned from the query.

Additionally, in a preferred embodiment, if the Sample Details option is selected in the View menu, a details panel will be displayed at the right of the window. This panel contains tabbed views that display detailed information about selected samples, including attributes, experiments, sample, and donor.

In a preferred embodiment of the present invention, the user can store and view information about when and how the sample set was created. This window contains the following: the date the sample set was created, the chipset used for the sample query the parameters that were used for the query, and any other relevant search criteria (for example, sort order). Preferably, this history is saved with the sample set.

In another preferred embodiment, as an alternate to an attribute-based sample query, a Genomics ID query mechanism is provided for creating a sample set from a list of known Genomics IDs.

Another embodiment of the invention provides for importing by attribute. The Import by Attribute option allows for importing samples based on a list of values for a specific attribute. These attributes must have been previously saved in a user-created text file. The result of the import will be a list of all samples whose values for the specified attribute match any of the values in the file.

Preferably the sample set can be saved to be reviewed at a later date or for use with the analyses. During the save process, the sample set is given a name and permissions can be set to limit who has access to the file.

In another preferred embodiment it is possible to save the search parameters of a query without saving any data along with them. In this way, the query can be accessed for later use. Unlike sample sets and genes which are saved to the workspace, the query templates are saved on the local disk. Saved sample sets can be re-opened for further analysis. Once saved, the contents of the results do not change, even when more samples that satisfy the query are added to the database. In order to make the sample set current, it is necessary to re-run the query.

The Sample Set preferably offers a number of menu options. These include the following: a File, New Sample Set Window tab which opens a new Sample Set window; File, Open Sample Set tab which opens the Select Sample Set window from which to open a saved sample set; a File, Open Query Template tab which opens the Open Query Template window in which to open a saved query template; a File, Save Sample Set As tab which opens the Save Sample Set As window where the sample can be saved; a File, Save Query Template As tab which opens the Save Query Template As window where the query template can be saved; a File, Save Selected Samples tab which opens the Save Sample Set As window where selected samples can be saved as a unique set; a File, Import Sample Ids tab which opens the Open window to import a list of genomics IDs from a previously saved text file; a File, Import by Attribute tab which opens the Import by Attribute window; a File, Export Sample Ids tab which opens the Save As window where a file in which to save the genomics IDs can be created; a File, Export tab which provides options for exporting the query results; a File, Invoke tab which provides options for accessing third-party applications in which to view the results; a File, Print tab which opens the Page Setup window for setting up the page layout and printing the results; a File, Union with Sample Set tab which opens the Select Sample Set window where a previously saved sample set can be selected, any samples in the selected sample set that are not already in the current sample set will be appended to it; a File, Exclude Sample Set tab which opens the Select Sample Set window where a previously saved sample set can be selected, any of this new set's members that are in the current sample set will be removed, the result is the set difference between the two sample sets; a File, Intersect Sample Set tab which opens the Select Sample et window where a previously saved sample set can be selected, only the members that are common to both gene sets will be displayed; and a File, Close tab which closes the sample set window.

Also preferably included are a Edit, Select All tab which selects all of the samples in the query results; an Edit, Remove Selected Samples tab which deletes selected samples; an Edit, Copy Selected Samples tab which copies selected sample(s) to the clipboard; an Edit, Paste Samples tab which pastes copied sample(s) from the clipboard; a View, Sample Details tab which, if checked displays details in the Results panel; a View, Select Display Attributes tab which opens the Select Display Attributes window where the user can select columns to display in the results; a View, Automatically Include Condition Attributes in Results tab which, if checked, includes the parameters that defined the search in the default display columns; a View, Add Normalization Support Column tab which includes Affy Normalization which adds a column indicating whether or not Affymetrix normalization is supported, a Gene Logic Normalization which adds a column indicating whether or not Gene Logic normalization is supported, and a Standard Curve Normalization which adds a column indicating whether or not standard curve normalization is supported.

The purpose of normalization is to allow for the comparison of the expression values reported from different gene chip experiments; therefore, if two different samples yield the same expression value for a gene fragment, there is reasonable confidence that the concentrations of mRNA transcripts for the fragment are the same in the two samples. Because of variations in the manufacturing process for the chips, as well as other factors, the unnormalized intensity values vary widely from one chip experiment to another for fragments with the same RNA concentration. There are many methods available to researchers to adjust for this variation. The application of the present invention preferably supports three of these methods; known as Affymetrix normalization, Gene Logic normalization, and standard curve normalization.

Affymetrix normalization is the method supplied within the Affymetrix gene chip analysis software. The average differential intensity values (or “AveDiffs”) produced by this software are the result of this normalization process. The normalized values are computed by multiplying the unnormalized values by a scale factor. The scale factor is the same for all values in an experiment, and is calculated as follows:

1. From all the unnormalized AveDiff values in the experiment, delete the largest 2% and the smallest 2% of the values. That is, if the experiment yields 10,000 expression values, order the values and delete the smallest 200 and the largest 200.

2. Compute the “trimmed mean,” equal to the mean of the remaining values.

3. Compute the scale factor SF=00/(trimmed mean).

Gene Logic normalization algorithm is based on the observation that the expression intensity values from a single chip experiment have different distributions, depending on whether small or large expression values are considered. Small values, which are assumed to be mostly noise, are approximately normally distributed with mean zero, while larger values roughly obey a log-normal distribution; that is, their logarithms are normally distributed with some nonzero mean. While Affymetrix normalization applies the same scale factor to all expression values in an experiment, Gene Logic normalization computes separate scale factors for “non-expressors” (small values) and “expressors” (large ones). The inputs to the algorithm are the Affymetrix-normalized AveDiff values, which are already scaled to set the trimmed mean equal to 100. The algorithm computes the standard deviation SD noise of the negative values, which are assumed to come from non-expressors. It then multiplies all negative values, as well as all positive values less than 2.0* SD noise, by a scale factor proportional to 1/SD noise. Values greater than 2.0* SD noise are assumed to come from expressors. For these values, the standard deviation SD log(signal) of the logarithms is calculated. The logarithms are then multiplied by a scale factor proportional to 1/SD log(signal) and exponentiated. The resulting values are then multiplied by another scale factor, chosen so there will be no discontinuity in the normalized values from unscaled values on either side of 2.0* SD noise. Standard curve normalization attempts to relate the original expression intensity values from the chip experiments to actual mRNA concentrations for each gene expressed in the sample. In order to do this, known concentrations of particular gene fragments must be “spiked in” to the sample RNA mixture before hybridizing it to the chips. (Bacterial genes are used for the spike-ins, so there will not be any additional RNA contribution from the sample donor.) The chip experiment yields intensity measurements for the spike-in gene fragments. Ideally, the intensities will increase linearly with concentration; therefore, if intensity is plotted vs. concentration, it should be possible to draw a straight line through the origin connecting the data points, and use its slope to infer the mRNA concentrations for the other gene fragments on the chip. In reality there are noise and non-linear effects which distort this relationship; but one can still draw a straight line through the origin that is the best fit to the data points. The straight line is known as the “standard curve.”

This normalization procedure is as follows:

1. Using identity link and gamma error, a generalized linear model is fit to the intensity versus concentration curve. A slope is determined, and applied to the raw intensity values by dividing by the slope to get a concentration. Only data which are called present are used in the fit.

2. These new concentration values for the spike-ins are entered into a logistic regression (with “A,” “U” or “N” called not present or 0, and “P” called present or 1) to determine a minimum sensitivity. The concentration corresponding to a logistic prediction of 0.7 is used as the sensitivity cutoff. If the logistic regression fails, the sensitivity value is estimated via interpolation at 0.7 times the difference between the highest concentration called absent and the lowest concentration called present, added to the highest concentration called absent.

3. The concentration values below 0 are reported as one half of the sensitivity cutoff.

4. Concentration values between 0 and the sensitivity value are reported as the average of the sensitivity cutoff and the raw value.

The concentration value (in picomoles) is reported as the expression value, rather than the intensity.

Standard curve normalization has the following implications for this version of the product: the Chipset options that are available for use will vary depending on the contents of the database the application has access to, including H.sapiens (Hu 42K), H.sapiens (HG_U95), M. musculus (Mu11K), M. musculus (Mu19K), M. musculus MG_U74), and R. norvegicus (RG_U34).

Another preferred aspect of the application of the present invention is the creation of a gene set. A gene set is a list of DNA fragments for which probe sets are provided on one or more gene chips. Users define gene sets by specifying a combination of query criteria that are applied to the gene database. Upon completion of the query, the present invention displays a list of genes satisfying the criteria; the user can then select specific genes from this list or save the gene set for use with the analyses.

Affymetrix fragments are the basic units for which the application of the present invention provides gene expression information. The present invention preferably does not provide access to the raw data for individual probes. Gene sets are created by performing a search of the gene index, the results of which can be saved for later use. The gene index is database of gene fragment annotations. Gene fragment annotations are obtained by linking the Affymetrix probe sets to UniGene clusters and, when possible, to known genes (found in NCBI's LocusLinks database), and then to protein, enzyme, pathway, functional, and other databases.

Affymetrix probe sets are tiled on gene chips that are species-specific (with the exception of the control probe sets). For example, the Human 42K chip set contains 42,000 probe sets based on 6,800 Human full-length mRNAs and 35K Human ESTs.

A preferred aspect of the present invention is the ability to query the gene sets. For example, the database can be searched for gene fragments related to the fatty acid metabolic pathway.

The first step in querying the gene set is to choose the appropriate subset of the gene index. The gene query enables a user to query the database for gene fragments of a particular species (that is, human, rat, or mouse). The next step is selecting the pathway. For this example, the metabolic pathway for fatty acids is used as the search parameter. The present invention preferably also allows for selecting search options, including: all of the following—when this option is selected, the search will be performed for only those conditions that satisfy all conditions; for example, the pathway “fatty acid metabolism” and the fragment type “_g (common groups);” any of the following—when this option is selected, the search will be performed for any of the search attributes selected, and results returned for any that are found. For example, results from both the pathway “fatty acid metabolism” and another parameter, such as fragment type “_g (common groups)” would be returned; and case sensitive—this option applies to attributes where a text value is typed in. In such cases, the capitalization of the results will exactly match what is entered, that is either lower or upper case.

In this preferred embodiment of the present invention, the user can specify the sort order of the results.

The results of the gene set query are preferably automatically displayed in the Results panel of the Gene Query window. This window preferably presents the following information: a statement above the results indicating the type of search performed, a statement indicating the total number of genes found in the query, and the number currently selected, and a table of genes returned from the query.

Preferably, if the Gene Details option is selected in the View menu, a details panel will be displayed. This panel contains tabbed views that display detailed information about selected results, including attributes and known gene.

Preferably the application of the present invention contains data for certain samples that have been run both on gene chips and on gels that provide restriction enzyme analysis of differentially expressed sequences (READS). The data from READS gels is preferably stored in a separate database.

Preferably an alternate way to create a gene set is to start with a nucleotide or protein sequence and search for Affymetrix fragments that match the sequence using BLAST. To distinguish the matching gene fragments in the results table for multiple BLASTs, an additional column, “Query Sequence,” is preferably displayed, showing the tag for the sequence that matched the fragment. If more than one query sequence matches the exemplar sequence of the same Affymetrix fragment, the one with the smallest p-value will be displayed. Once a gene set is created from BLAST, it can be manipulated and saved just like any other result.

Another preferred aspect of the application of the present invention is the ability to import by attribute. Import by Attribute allows for importing Affymetrix fragments based on a list of values for a specific attribute. These attributes must have been previously saved in a user-created text file. The result of the import will be a list of all Affymetrix fragments whose values for the specified attribute match one of the values in the file. The GenBankID import is a special case where Affymetrix fragments can be imported according to the values of the Exemplar Seq: Accession attribute.

The gene set preferably can be saved for later use or for use with the analyses. Saved gene sets can be re-opened for further analysis. Once saved, the contents of the results do not change, even when more genes that satisfy the query are added to the database. In order to make the gene set current, it is necessary to re-run the query. If the user wishes to retain the original results, save the new results under another name.

It will be appreciated that there are a variety of menu options that are available for use with the gene set query, including: a File, New Gene Set Window tab which opens a new Gene Query window; a File, Open Gene Set tab which opens the Select Gene Set window from which a previously saved gene set can be opened; a File, Open Query Template tab which opens the Open Query Template window from which a saved query template can be opened; a File, Save Gene Set As tab which opens the Save Gene Set As window in which the gene set can be saved; a File, Save Query Template As tab which opens the Save Query Template As window in which the query template can be saved; a File, Save Selected Genes tab which opens the Save Gene Set As window in which selected genes can be saved as a unique set; a File, Import Gene Ids tab which opens the Open window where it is possible to browse to find previously saved Affymetrix fragment name IDs to import; a File, Import by Attribute tab which opens the Import by Attribute window; a File, Export Gene Ids tab which opens the Save As window where a file can be created in which to save the gene Ids and which can then be used with other, third-party applications; a File, Export tab which provides options for exporting the results; a File, Invoke tab which provides options for accessing third-party applications in which to view the results; a File, Print tab which opens the Page Setup window for setting up the page layout and printing the results; a File, Union with Gene Set tab which opens the Select Gene Set window in which a previously saved gene set can be selected, any genes in the selected set that are not already in the current set will be appended to it; a File, Exclude Gene Set tab which opens the Select Gene Set window in which a previously saved gene set can be selected, any of this new set's members that are in the current gene set will be removed, the result is the set difference between the two gene sets; a File, Intersect Gene Set tab which opens the Select Gene Set window where a previously saved gene set can be selected, only the members that are common to both gene sets will display; and a File, Close tab which closes the gene set window.

The gene set query preferably also includes an Edit, Select All tab which selects all of the results in the gene set; an Edit, Remove Selected Genes tab which removes selected genes from the gene set; an Edit, Copy Selected Genes tab which copies selected gene(s) to the clipboard; an Edit, Paste Genes tab which pastes copied gene(s) from the clipboard.

The gene set query preferably also includes a View Gene Details tab which, if checked, displays details in the results panel; a View, Select Display Attributes tab which opens the Select Display Attributes window in which columns for displaying the results can be selected; a View, Automatically Include Condition Attributes in Results tab which, if checked, includes the parameter(s) that defined the search in the default columns that are displayed; a View, Blast Output tab which exports the BLAST results to the default Web browser, where additional BLAST information (sequence alignment) can be viewed; and a View, Add READS Link Column tab.

The gene set query preferably also includes the ability to select gene chips. The Chipset options that are available for use will vary depending on the contents of the database the application has access to, including H.sapiens (Hu 42K), H.sapiens (HG_U95), M. musculus (Mul 1K), M. musculus (Mu19K), M. musculus (MG_U74), and R. norvegicus (RG_U34).

Another preferred embodiment of the application of the present invention is a gene signature analysis of a sample set which extracts two sets of gene fragments from all of the gene fragments represented in the sample set's chipset: those that are consistently expressed within the sample set, and those that are consistently not expressed.

In order to perform the gene signature analysis, it is necessary to quantify the “consistency” of expression as two threshold percentages, one for the “present” set, the other for the “absent” set. Consistency of expression is a measure of how frequently a gene (Affymetrix fragment) is expressed, or not expressed, in a sample set. For example, if there are 5 samples in the sample set, and the user sets the present and absent threshold percentages to 80% and 80%, respectively, then the gene signature analysis computes one set of genes that are present in at least 4 out of 5 samples, and another set which are absent in at least 4 of 5 samples.

For computing the Gene Signature Analysis, Affymetrix fragments that have “marginal” calls for a particular sample are treated the same as “absent” fragments. Fragments that have “unknown” calls are ignored in the gene signature computation. If, for a particular Affymetrix fragment, p, m, and a are the numbers of samples for which the fragment was present, marginal, and absent, respectively, then the fractions p/(p+m+a) and (m+a)/(p+m+a) are computed; these fractions are compared against the present and absent threshold percentages to determine if the fragment belongs to either of the gene signature gene sets.

For example, suppose the data warehouse of the present invention contained the present/absent/marginal/unknown call values shown in the table below, for the sample set S={s1, s2, s3, s4} and the genes {g1, g2, g3, g4, g5, g6, g7, g8, g9}. (In reality there would be data for thousands of genes, but only nine genes are shown for illustration.) At the bottom of the column for each gene the percentages computed from the numbers of present, absent, and marginal calls for each gene across sample set S are shown. Suppose that the present and absent threshold percentages were both set to 75%. Then for this sample set, the gene signature operation returns a “present Gene Set” containing genes {g1, g2, g3, g4}, and an “absent Gene Set” containing {g5, g6, g7, g9}.

The gene signature analysis also computes the mean, median, and standard deviation for each gene in the present and absent sets. The user can select any or all of these values to be displayed in the gene signature results.

The curves for the gene signature are computed as follows:

1. Compute the present gene counts for each sample in the sample set.

2. Order the samples by present gene count in ascending order.

3. Initialize P to the set of present genes in the first sample. The height of the first point in the curve is the number of genes in P.

4. Intersect P with the set of present genes in the second sample, and repeat for each sample in the sample set. The heights of the successive points in the curve are the number of genes in P after each intersection step. The X axis component of each point is the index of the corresponding sample in the sorted sample set.

5. Repeat steps 1 through 4 for the absent genes, and plot intersection set counts on separate graphs.

In a preferred aspect of the present invention, the gene signature curve does not take into account the percentage thresholds specified. The gene signature curve works as a robustness test for the gene signature. The purpose of the gene signature curve is to show that the Gene Signature operation had enough samples to reach stability, that is, the count after intersecting does not change significantly. The method used to produce the gene signature present and absent gene sets is not the same as the algorithm used to compute the gene signature curve. The gene signature computation utilizes a threshold percentage to obtain the Present/Absent Gene Sets, while the curve computation does not. Furthermore, U (unknown) and N (no expression data—that is, samples with missing chips) calls play a crucial role in producing discrepancies between the gene signature and the gene signature curve.

Note that the calculation algorithm does correct for partial chip sets and missing data by including only the samples for which there are expression data. Thus, all genes are included in the Present Gene Set, even though each of them is only called present in a portion of the samples.

In the present invention, the “Number of Genes” values equal to zero are NOT plotted. This is the reason that the maximum number of samples shown on the x-axis may differ from the number of samples in the sample set, and may even differ between the present and absent gene signature curves. The algorithm first orders the samples by the present count in ascending order, then initializes P to the set of present genes in the first sample. The height of the first bar in the curve is the number of genes in P. P is then intersected with the set of present genes in the second sample, and the number of genes remaining in P is shown as the height of the second bar in the curve. This process is repeated for each sample in the sample set. The U (unknown) and N (no data for sample) calls play a crucial role in producing these “irregularities.” This example shows how the seeming irregularities are produced by these two algorithms on the same data. Hence, values can be obtained where the last element in the histogram chart is not the same as the size of the gene set, as well as having the x-axis not equal to the size of the sample set.

As an example of computing a gene signature, using a “Breast Cancer” sample set created previously, a gene signature can be computed where both the present and absent thresholds are set to 75%. The Breast Cancer sample set was derived using the H.sapiens (HG_{—}95U) chipset, the Organ:Breast, and the Morphology:Infiltrating Duct Carcinoma search parameters.

There are a variety of ways in which the result of the gene signature analysis can be displayed. After the analysis is complete, the results are preferably displayed in the Summary tab of the Gene Signature Analysis window. This window presents the following information: a panel displaying the number of gene fragments in the Present Gene Set, a panel displaying the number of gene fragments in the Absent Gene Set, and the name of the sample set and the number of samples it contains.

Preferred default summary columns which include the following: GenomicsID, Experiment(s), Total Present Calls, Total Absent Calls, Total Unknown Calls, Present Calls (Present Gene Set), Unknown Calls (Present Gene Set), Absent Calls (Absent Gene Set), and Unknown Calls (Absent Gene Set).

Preferably, the Gene Signature History is displayed. This presents information about the thresholds used to compute the analysis, the date and time the analysis was performed, and the version of the Runtime Engine (RTE) used for the analysis.

Preferably, if the Show Details Panel option is selected in the View menu, a details panel will be displayed. This panel contains views that display detailed information about selected samples, including Sample Detail, Attributes, Experiments, Sample, and Donor.

In a preferred aspect of the present invention, the gene signature curve tab provides several options, including: Number of Fragments vs. Number of Samples and Number of Fragments vs. Threshold Percentage.

The Number of Fragments vs. Number of Samples option displays a pair of gene signature curves, one for the present gene set and one for the absent gene set. This display is designed to give the user a visual sense of whether the sample set is large enough to generate a valid gene signature. The number of samples in the gene signature curve may differ from the number of samples in the sample set.

The Number of Fragments vs. Threshold Percentage option displays the counts of the present and absent genes as a function of the threshold percentage. For example, if both thresholds were set to 90%, which means that qualified fragments should be present or absent in 76 out of 84 samples, the number of fragments in the present and absent set would be approximately 10,000 and 30,000 respectively. If the thresholds were set at 75% (less stringent) the sets grow to approximately 13,000 and 39,000 respectively.

Detailed information about the gene fragment results are preferably displayed in the Gene Set Results tab. These include the Present Gene Set results, the Absent Gene Set results, the number of genes in the Present or Absent Gene set, depending on which tab is selected, a statement about the type of normalization used, and a table of gene results in both the Present Gene Set or Absent Gene Set view.

Preferably, the present invention includes a Show Details option which, if selected, will display detailed information about selected gene fragments, including Affy Fragment Details, including Attributes and Known Gene; Sample Details, including Attributes, Experiments, Sample, and Donor; Sequence Cluster; and Plot.

The Sequence Cluster tab preferably presents a view of a gene fragment in the context of the UniGene cluster it is classified under. By selecting a row in the main results window and then selecting this tab, it is possible to view a table with the expression values of all gene fragments in the same UniGene cluster over the corresponding sample or sample set.

The Plot aspect of the present invention preferably displays a visual representation of expression values for the selected Affymetrix fragment. The plot shows lines or circles (depending on the user's preference) corresponding to the expression values for individual samples, overlaid with a translucent box plot in which the ends of the box represent the user-specified percentile values.

The plot also displays multiple rows for a gene, one per input sample set; these are paired with bar graphs showing the percentage of samples in each sample set in which the gene is called present. Vertical bars are displayed at the median, the lower quartile minus 1.5 times the interquartile range, and the upper quartile range plus 1.5 times the interquartile range. Assuming a normal distribution, the extreme bars are located approximately 3 standard deviations away from the median. Their locations are independent of the user-specified percentile values. The X axis of the plot shows graduated markers indicating expression intensity.

A preferred aspect of the present invention is the ability to view pathways. The Pathway Viewer tab presents a pathway display where expression values are overlaid on known metabolic or enzymatic pathways.

Another preferred aspect of the present invention is the ability to viewing chromosome maps. The Chromosome Viewer tab presents a display that renders expression values over a chromosome map. The chromosome diagram preferably provides a statement about the number of markers, and the number of matches displayed; that is, the total number of Affymetrix fragments on the chromosome, and the number from the current gene set; a statement about the display option: “Mean” values were selected in the example; a table containing results data, which table can be manipulated just like other result tables; a panel displaying the chromosome image, along with a vertical axis that displays the expression values.

In this preferred embodiment, the Median Values option displays Median Expression values for the sample set, mapped to Minus or Plus strand; the Mean Values option: displays Mean Expression values for the sample set, mapped to Minus or Plus strand; the Raw Expression Values option displays Expression Values for all Samples; and the Call Values option displays the Call Values for all Samples.

Preferably it is possible to save any or all of the results as a unique gene set. This gene set can then be used with other analyses.

In another preferred embodiment of the application of the present invention, a Set Gene Mask option permits filtering of the gene set. The gene mask allows for either intersecting gene sets to reveal shared genes, or for displaying the differences between gene sets.

The results produced from the analyses preferably can be exported to a variety of third-party applications, including the Eisen Cluster Tool, GeneSpring, and Partek Pro 2000.

Preferably there are a variety of menu options that are available for use with the gene signature analysis, including: a File, New Opens option which opens a new gene signature analysis window; a File, Open option which opens the Select Gene Signature window from which a saved gene signature can be opened; a File, Save Gene Signature option which opens the Save Gene Signature As window in which the gene signature can be saved; a File, Save Gene Set option which allows for saving the results as a gene set; a File, Save Selected Genes option which opens the Save GeneSet As window in which selected gene fragments can be saved as a unique gene set; a File, Export option which provides options for exporting the results; a File, Invoke option which provides options for accessing third-party applications in which to view the results; a File, Print option which opens the Page Setup window for setting up the page layout and printing the results; and a File, Close option which closes the Gene Signature Analysis window.

Preferably the gene signature analysis also includes: a View, Compute Form option which accesses the Compute tab; a View Summary option which accesses the Summary tab; a View, GS Curve option which accesses the gene signature curve tab; a View, Gene Set Results option which accesses the Gene Set Results tab; a View, Pathway Viewer option which accesses the Pathway Viewer tab; a View, Chromosome Viewer option which accesses the Chromosome Viewer tab; a View, Show Details Panel option which, if checked, displays details in the Summary or Results panel; a View, Select Display Attributes option which opens the Select Display Attributes window; a View, Gene Set Mask Add/Remove Mask option which opens the Add/Remove Gene Set Mask window in which to add or remove masks to gene sets; a View, Remove Selected Genes option which removes the selected genes from the currently displayed results; a View, Remove Unselected Genes option which removes the unselected genes from the results; a View, Reset to Original Gene Set(s) option which resets the results to their original state; a View, Sort By option which sorts the results; a View, Options option which opens the gene signature view options window for selecting viewing options; and a View, Plot Options option which opens the Plot Option window where display options for the plot can be selected.

In another preferred embodiment of the present invention, the application can perform a gene signature differential analysis. A gene signature differential analysis compares the results of two sample sets. Using these two sample sets, the analysis computes two new sets of gene fragments.

A gene signature differential analysis compares two sample sets (which must have been previously computed and saved). The analysis derives two new sets of gene fragments: those that are in both the first samples set's present gene set and the second's absent gene set and those that are in both the first sample set's absent gene set and the second's present gene set.

There are preferably several components of presentation of the results of the signature differential analysis, including the names of the two input sample sets, the size of the sample sets used, and the thresholds used to compute the gene signatures; a table summarizing the number of gene fragments in the two present sets: Present only in <Gene Set 1>, Present only in <Gene Set 2>; and a History panel that records the date and time of the analysis and the version of the runtime engine used.

Detailed information about the gene fragment sets for the data the user has have selected are preferably displayed in the Gene Set Results tab. The information presented in this view preferably includes: a tab that displays gene sets that are Present only in <1 st Gene Set>; a tab that displays gene sets that are Present only in <2nd Gene Set>; a tab that displays gene sets that are Present in both (gene sets); a tab that displays gene sets that are Absent in both (gene sets); a statement of the number of rows in the results and the type of normalization used; and a table of genes in the selected tab view.

Preferably, if the Show Details Panel option is selected in the View menu, a details panel will be displayed. This panel contains views that display detailed information about selected samples, including Sample Detail, Attributes, Experiments, Sample, and Donor; Sequence Cluster, and Plot.

Preferably one can further refine the data content of the Gene Set Results tab by selecting viewing options. These options include Show Affy Fragments only which, ff selected, user-specified attributes of qualified Affymetrix fragments will be displayed; Aggregate (per Sample Set) Values which, if selected, expression value statistics for each Affymetrix fragment will also be displayed; Expression and Call values (One Row per Gene) which, if selected, the results table displays one row per gene which contains the present/absent call and quantitative expression value for the fragment across all samples in the sample set; and Expression and Call values (One Row per Gene per Sample) which, if selected, the result table displays one row per fragment per sample including the actual present/absent call and the quantitative expression value for the fragment.

The application of the present invention also preferably includes the ability to viewing pathways. The Pathway Viewer tab presents a pathway display where expression values are overlaid on known pathways.

One can further preferably refine the content that the Pathway Viewer tab displays by selecting viewing options, which include Median Values for Sample Sets which, if selected, the median expression levels will be displayed for each Affymetrix fragment in the selected gene set that overlaps the pathway, over all samples in the input sample sets; Mean Values for Sample Sets which, if selected, the mean expression levels will be displayed for each Affymetrix fragment in the selected gene set that overlaps the pathway, over all samples in the input sample sets; Raw Expression Values (Selected Affy Fragments Only) which, if selected, the raw expression levels will be displayed for each Affymetrix fragment in the selected gene set that overlaps the pathway, over all samples in the input sample sets; and Raw Expression Values (All Affy Fragments in Pathway) which, if selected, the raw expression levels will be displayed for all Affymetrix fragments that map to the pathway, regardless of the gene set selected, over all samples in the input sample sets.

The application of the present invention also preferably includes the ability to viewing chromosome maps. The Chromosome Viewer tab presents a display that renders expression values over a chromosome map.

One can further preferably refine the content that the Chromosome Viewer tab displays by selecting viewing options, which include Median Values for Sample Sets which, if selected, median expression values for each gene fragment across all samples in the gene signature sample sets will be displayed for the chromosome; Mean Values for Sample Sets which, if this option is selected, mean expression values for each gene fragment across all samples in the gene signature sample sets will be displayed for the chromosome; Raw Expression Values for Samples which, if this option is selected, raw expression values for each gene for each sample in the selected sample sets will be displayed; and Call Values for Samples which, if this option is selected, call values will be displayed.

The gene signature differential can preferably be saved for later use. It is also preferably possible to save any or all of the resulting set as a unique gene set. This gene set can then be used with other analyses. Various options are preferably included in saving a gene set, including Present Only in <“1st Gene Set”>, Present Only in <“2nd Gene Set”>, Present in both, and Absent in both.

The gene signature differential menu options include a variety of menu options, including: a File, New tab which opens a new gene signature differential analysis window; a File, Open tab which opens the Select GeneSigDiff window from which a previously saved gene signature differential can be opened; a File, Save GS Differential tab which opens the Save GeneSigDiff As window where the gene signature differential can be saved; a File, Save Gene Sets tab which opens the Save Gene Set As window; a File, Save Selected Genes tab which opens the Save Gene Set As window in which gene fragments selected in the table can be saved as a unique gene set; a File, Export tab which provides options for exporting the results; a File, Invoke tab which provides options for accessing third-party applications in which to view the results; a File, Print tab which opens the Page Setup window for setting up the page layout and printing the results; and a File, Close tab which closes the Gene Signature Differential Analysis window.

The gene signature differential menu options preferably also include: a View, Compute Form tab which accesses the Compute tab; a View, Summary tab which accesses the Summary tab; a View, Gene Set Results tab which accesses the Gene Set Results tab; a Pathway Viewer tab which accesses the Pathway Viewer tab; a Chromosome Viewer tab which accesses the Chromosome Viewer tab; a Show Details Panel tab which, if checked, displays details in the Results panel; a View, Select Display Attributes tab which opens the Select Display Attributes window; a View, Gene Set Mask Add/Remove Mask tab which opens the Add/Remove Gene Set Mask window in which to add or remove masks to gene sets; View, a Remove Selected Genes tab which removes the selected genes from the currently displayed results; a View, Remove Unselected Genes tab which removes the unselected genes from the results; a View, Reset to Original Gene Set(s) tab which resets the results to their original state; a View, Sort By Sorts tab which sorts the results; a View, Options tab which opens the Gene Signature Differential Options window for selecting viewing options; and a View, Plot Options tab which opens the Plot Option window where display options for the plot can be selected.

The application of the present invention also preferably includes the ability to perform a fold change analysis. A Fold Change Analysis compares the mean expression levels of each gene fragment in a chipset between a control sample set and an experimental sample set to compute a fold change ratio. The Fold Change Analysis quantifies the change in expression for differentially expressed genes between pairs of sample sets. After computing the fold changes for each fragment, the fragments are classified by fold change value.

A Fold Change Analysis operates on quantitative expression values. It computes, for each of a set of selected gene fragments, the ratio of the geometric means of the expression intensities in a control sample set and an experimental sample set. The fold change is equal to this ratio. If the ratio is less than one, and the user has elected to display fold changes with magnitudes and directions, then the fold change magnitude is the reciprocal of the ratio, with a “down” direction. Multiple fold change comparisons may be run in parallel between different experimental sample sets and matched control sample sets. The analysis categorizes gene fragments by the fold change of their mean expression values between each pair of sample sets, and reports detailed expression information for those fragments whose fold changes fall within a user-specified range, or for fragments in a user-specified gene set. Confidence limits and p-values are also calculated when possible. The algorithm is based on a two-sided Welch modified two-sample t-test. It assumes that the logarithms of the expression intensities for each sample set are normally distributed, and that the variance of each control sample set may differ from the variance of the experimental set it is being compared to.

Note that the p-values are not corrected for multiple comparisons. The null hypothesis used for the t-test is that the population means for the logs of the expression values are the same in the two sample sets. The alternative hypothesis is that the means are different. The p-value reported is an estimate of the probability that a difference of means (and thus a fold change) as extreme as that observed could be obtained under the null hypothesis.

Confidence limits on the fold change value are calculated according to the same set of assumptions. By default, 95% confidence limits are computed; a different confidence level can be specified by the user. The upper and lower 95% confidence limits reported are the estimated bounds of the interval for which, under the above assumptions, there is a 95% probability that the actual ratio of population means falls within the interval. Both sample sets must have more than one sample. If one or both of the sample sets has only one member, then confidence limits and p-values cannot be calculated, though a fold change is still reportable using the algorithm described below.

Fold change is calculated on a per fragment basis: that is, the fold change algorithm is applied to each fragment separately. Users preferably have the option to choose Gene Logic normalized, standard curve normalized, or Affymetrix normalized expression values for the analysis, but the same normalization must be used across all samples and genes. A floor is applied to the expression values with Gene Logic or Affymetrix normalization; the floor value used is based on a noise parameter Q, which depends on the type of normalization chosen.

For Gene Logic normalized expression values (“GL expression”), each chip has a standardized noise level Q equal to 10. More precisely, it estimates the distribution of the noise on each chip as part of the Gene Logic normalization, and recalculate the expression values so that the standard deviation of GL expression values near 0 is equal to 10.

For Affymetrix normalized expression values, the analysis uses the actual noise value Q=RawQ*SF calculated for each chip experiment by the Affymetrix software and stored in the database.

The user preferably also has the option to compute the fold change using only samples for each gene for which the gene is called present. When this option is selected, the numbers of samples nx and ny for each sample set will vary for different genes, and it may not be possible to compute p-values and confidence limits for every gene. The inputs to the algorithm are two sample sets (X and Y) and one gene set, along with the user-specified confidence level CL (between 0 and 100%, defaulting to 95%).

Fold Change Algorithm

For sample set X and a gene fragment f in the gene set, do the following:

1. First apply a floor value to the expression data. Let e_{fi} be the normalized expression value for fragment f in sample i.

If Gene Logic normalization is used, set e_{fi} to max(e_{fi}, 20).

If Affymetrix normalization is used, set e_{fi} to max(e_{fi}, 2*SF_{fi}*RawQ_{fi}), where RawQ_{fi} and SF_{fi} are the RawQ and scale factor parameters from the chip experiment on the chip containing fragment f, for sample i. If the resulting e_{fi}<20, set e_{fi} to 20.

If standard curve normalization is used, leave e_{fi} alone; do not apply a floor value.

2. Given expression levels {e_{fi}: i=1, 2, . . . , nₓ} across nₓ samples in sample set X, calculate the logs: xᵢ=1n(e_{fi}).

3. Calculate the mean(x), i.e., mean(x)=(sum over i of xᵢ)/nₓ.

4. Calculate the variance(x), i.e., var(x)=(sum over i of (xᵢ−mean(x))²)/(nₓ−1).

5. Repeat steps 1-4 for sample set Y.

6. Calculate a t statistic:

t=(mean(x)−mean(y))/s

where s=sqrt(var(x)/nₓ+var(y)/n_{y})

7. The computation of the p-value and confidence limits requires the cumulative T probability distribution function Pt(t, DF) and the inverse function tInverse(p, DF). Compute the (non-integral) degrees of freedom parameter:

DF=1/(ci²/(nₓ−1)+((1−c)²)/(n_{y}−1))

Where c=var(x)/(nₓ*s²)

8. Calculate the p-value by:

Pval=Prob(|T|>t)=2*(1−Pt(t,DF))

where Pt(t, DF) is the cumulative T distribution with DF degrees of freedom and t is the statistic specified above.

9. Compute the fold change ratio FC and upper and lower confidence limits. Given the user specified confidence level CL, compute:

TI=s*tInverse((100+CL)/200, DF)

Now the fold change and confidence limits are calculated using:

m mean(x)−mean(y)

FC=exp(m)

Lower confidence limit=exp(m−TI)

Upper confidence limit=exp(m+TI)

The fold change direction is reported as “up” if FC>1 and “down” if FC<1; the fold change magnitude is FC if FC>1 and 1/FC if FC<1.

After computing the fold changes for each fragment between the control and experiment sample sets, the fragments are classified by fold change value, and a summary report is produced showing the counts of fragments with fold changes within certain ranges. Typically the user is interested in all gene fragments that have fold change magnitudes greater than a certain value. Fragments for which all samples in both sample sets return an absent call may be included in or excluded from the counts.

Given control and experiment sample sets and a gene G, the fold change for G is computed as the ratio of the geometric means of the intensities for gene G over the two sample sets. If the user selects the toggle “Use only samples where gene is present,” then the intensities for the samples where G is called absent are excluded from the geometric mean calculation; otherwise all intensities are included. In both cases, a floor value is applied to the intensities, depending on the normalization selected. If “Gene Logic” normalization is used, the floor value is 20 (that is, all intensities less than 20 are replaced with 20 before calculating the geometric means). If “Affy” normalization is selected, the floor value applied to the intensities from a particular chip experiment is twice the Q value computed for that experiment (that is, a different floor value is used for each sample/chip pair).

Confidence limits are calculated using a two-sided Welch modified t-test on the difference of the means of the logs of the intensities. The Welch form of the t-test is used because variances are generally unequal between the two groups of samples being compared. The logs of the intensities are assumed to come from a normal distribution. The confidence bounds are no longer symmetric about the fold change estimate on an additive scale; however, they are symmetric about the fold change estimate on a multiplicative scale, which is the appropriate type of scale for ratios (such as fold changes).

Preferably, the results of the fold change analysis can be displayed in a summary which presents a summary of the number of genes in each fold change bracket and the direction of the fold changes between the control and experimental set(s). It preferably displays the following information: a list of all of the control sample sets and the number of samples in each; a list of all of the experimental samples and the number of samples they contain; a check box which the user may select to include in the gene counts fragments that were absent in both the experimental and control sample sets; a table listing the number of gene fragments with fold changes in the following ranges: greater than 100; between 10 and 100; between 5 and 10; between 4 and 5; between 3 and 4; between 2 and 3; between 1 and 2; and with no change.

The numbers are preferably broken down in the following manner: the number of fold changes “up” in the experimental versus the control set; the number of fold changes “down” in the experimental versus the control set; and the total of all changes in the experimental versus control set.

Preferably the user can obtain more specific data about the fold change analysis results, including filtering gene fragments, viewing the results, viewing pathways, and viewing chromosome maps.

The Filtering Gene Fragments option allows for filtering the reported genes using a previously saved gene set.

The data content of the Gene Fragments (or, in other words, the Gene Set Results) can preferably further be refined by selecting viewing options, including magnitude and direction which displays the fold changes and the confidence, with values <1 changed to their reciprocals, along with extra columns showing the direction of the change (up or down); ratio (<1.0 if downward) which displays all fold changes and confidence limits as ratios; Show Raw Expression and Call Values which, if selected, quantitative expression values and present/absent calls are displayed, for each gene fragment and sample; and Show Mean, SD for Each Sample Set which, if selected, means, medians, and standard deviations for each sample set will be displayed.

The application of the present invention also preferably includes the ability to view pathways with regard to selected gene fragments. The Pathway View tab presents a pathway display where expression values are overlaid on known pathways. The content that the Pathway View tab displays can be refined further by selecting viewing options, including Fold Changes for Sample Sets which, if selected, the fold change values for each Affymetrix fragment in the selected gene set that overlaps the pathway will be displayed; Mean Values for Sample Sets which, if selected, the mean expression levels will be displayed for each Affymetrix fragment over all samples in each input sample set; Median Values for Sample Sets which, if selected, the median expression levels will be displayed for each Affymetrix fragment over all samples in each input sample set; Raw Expression Values for Samples which, if selected, the raw expression levels will be displayed for each selected Affymetrix fragment; All Affy Fragments in Pathway which, if selected, all gene fragments which overlap the pathway will be displayed; and Selected Affy Fragments Only which, if selected, only gene fragments selected in the Filter Gene Fragments panel will be displayed.

The application of the present invention also preferably includes the ability to view chromosome maps which present a display that renders expression values over a chromosome map. The content that the Chromosome View tab displays can be further refined by selecting viewing options, including Fold Changes which, if is selected, fold change values will be displayed; Median Values which, if selected, median values will be displayed; Mean Values which, if selected, mean values will be displayed; Raw Expression Values for Samples which, if this option is selected, raw expression values will be displayed; and Call Values for Samples which, if selected, call values will be displayed.

The fold change analysis preferably can be saved for future use.

Preferably there are a variety of menu options that are available for use with the fold change analysis, including a File, New tab which opens a new Fold Change Analysis window; a File, Open tab which opens the Select Fold Change MultiSet window from which a previously saved fold change can be opened; a File, Save Fold Change tab which opens the Save Fold Change MultiSet As window in which to save the fold change; File, Save Gene Set tab which opens the Save Gene Set As window where the result gene set can be saved; a File, Save Selected Genes tab which opens the Save Gene Set As window where selected gene fragments can be saved as a unique gene set; a File, Export tab which provides options for exporting the results; a File, Invoke tab which provides options for accessing third-party applications in which to view the results; a File, Print tab which opens the Page Setup window for setting up the page layout and printing the results; and a File, Close tab which closes the Fold Change Analysis window.

Preferably the fold change analysis menu also includes a View, Gene or Sample Details tab which, if selected, displays the details of a selected gene fragment or sample; a View, Select Display Attributes tab which opens the Select Display Attributes window; a View, Add READS Link Column tab which opens the Select Study window; a View, Gene Set Mask Add/Remove Mask tab which opens the Add/Remove Gene Set Mask window in which to add or remove a gene set mask to the results; a View, Remove Selected Genes tab which removes the selected genes from the currently selected results; a View, Remove Unselected Genes tab which removes the unselected genes from the results; a View, Reset to Original Gene Set(s) tab which resets the results to their original state; a View, Sort By tab which sorts the results; a View, Options tab which opens the Fold Change View Options window for selecting viewing options; and a View, Plot Options tab which opens the Plot Option window where display options for the plot can be selected.

In another preferred embodiment of the present invention, the application can perform an Electronic Northern Analysis. An Electronic Northern Analysis (ENorthern) takes a user-defined gene set and one or more sample sets as input. The range of expression levels is reported for each gene fragment in the gene set across each sample set, for all of the samples with user-specified present/absent calls. The range of expression values for a gene in an ENorthern analysis is reported as a pair of user-selected percentiles over the values for the samples in each sample set. By default, the values at the 25th and 75th percentiles over each sample set are shown. The user may select different percentiles. For example, the user may choose to view the 0th percentile (the minimum expression value) and the 100th percentile (the maximum) for each sample set. In addition to the user-specified percentiles, the median expression value (the 50th percentile) is always reported.

An Electronic Northern Analysis (or E Northern) takes as input a user-defined gene set and one or more sample sets, and reports the range of expression levels for each Affymetrix gene fragment in the gene set across each sample set, over all the samples with user specified present/absent call values. The range is reported using percentile values, with the upper and lower percentile levels U and L specified by the user. If the user chooses U to be 100 and L to be 0, the analysis reports the maximum and minimum expression values over the selected samples. If the user chooses U =75 and L =25, the upper and lower quartile values are reported. The median value is reported as well.

The E Northern is computed as follows for each sample set:

1. The user's selection in the E Northern Options dialog is used to determine how samples with absent and marginal calls will be used in the computations. If “Include Present calls only in computation” is selected, only samples with present calls are used in the percentile and present score computations; marginal calls are treated the same as absent calls and are included in the absent score. If “Include Present and Marginal calls in computation” is selected, samples with either present or marginal calls are included in the percentile and present score computations. If “Include Present, Marginal, and Absent calls in computation” is selected, samples with present, marginal or absent calls are used to compute the percentiles, and marginal calls are included in the present score.

2. For each gene fragment in the user-specified gene set, present and absent scores are computed by counting the numbers of Present and Absent calls for the samples in the given sample set, and dividing each count by the total number of samples that have expression data for the gene fragment. Samples with Unknown and Null calls are omitted and are not included in the total count of samples. The result is reported as a fraction in the tabular display (e.g., 17/22) and as a percentage in the E Northern plot.

3. For each gene fragment, the percentile and median values are computed over the samples with user-selected call values. The expression values for these samples are first sorted in ascending order. This generates a rank order R for each expression value, R=1 . . . N; where N is the number of selected samples. Define X R as the expression value with rank order R.

4. Three percentile values are computed: the 50th percentile (i.e., the median), and the two user specified percentiles L and U. Recall that the Pth percentile of a set of values is the value X such that P percent of the values in the set are less than X.

5. Let M=1 +((P/100)*(N−1)).

6. If M is an integer, the Pth percentile is X M, the expression value with rank order M.

In this case, the plot will return the expression values which are one rank higher than what the table returns for the upper and lower percentiles. The data in the table is more accurate than the plot.

7. If M is not an integer, the Pth percentile is obtained by interpolating between the values X M and X M+1. Let F be the fractional part of M. Then the Pth percentile is computed as

XM+F*(XM+1−XM)

8. The above calculation is performed for P=L, P=50, and P=U.

The ENorthern analysis is preferably computed using one or more sample sets and one or more gene sets. The gene set(s) can be either an existing gene for a gene set defined by using a gene signature differential.

Detailed information about the gene fragments in the E Northern results is preferably displayed in the Results tab. Preferably, this information includes a statement of the following: the number of rows, the upper and lower percentiles used, the normalization used, and the call types (present, absent or marginal) used to compute the percentiles; and a table of genes.

Preferably the ENorthern provides a Show Details Panel which, if selected, displays detailed information about selected gene fragment, including Affy Fragment, which includes Attributes and Known Gene data; Sample Details, which include Attributes, Experiments, Sample, and Donor data; Sequence Cluster; and Plot.

Preferably, the data content of the Results can be further refined by selecting viewing options, including Include Present calls only in computation, which, if selected, the percentiles are computed using expression values that are associated only with Present calls; Include Present and Marginal calls in computation, which, if selected, the percentiles are computed using expression values that are associated with Present and Marginal calls; and Include Present, Marginal, and Absent calls in computation, which, if selected, the percentiles are computed using expression values that are associated with Present, Marginal, and Absent calls.

The E Northern Analysis can preferably be saved for later use.

Preferably there are a variety of menu options that are available for use with the E Northern analysis, including a File, New tab which opens a new Electronic Northern Analysis window; a File, Open tab which opens the Select ENorthern window from which a previously saved E Northern analysis can be opened; a File, Save ENorthern tab which opens the Save ENorthern As window where the E Northern analysis can be saved; a File, Save Gene Set tab which opens the Save Gene Set As window in which the gene set used for the E Northern can be saved; a File, Save Selected Genes tab which opens the Save Gene Set As window in which selected gene fragments can be saved as a unique gene set; a File, Export tab which provides options for exporting the results; a File, Invoke tab which provides options for accessing third-party applications in which to view the results; a File, Print tab which opens the Page Setup window for setting up the page layout and printing the results; and a File, Close tab which closes the Electronic Northern window.

The menu options that are available for use with the E Northern analysis preferably also includes a View, Compute Form tab which accesses the Compute tab; a View, Results tab which accesses the Results tab; a View, Show Details Panel tab which, if checked, displays details in the Results view; a View, Select Display Attributes tab which opens the Select Display Attributes window where columns to display in the results can be selected; a View, Sort By tab which sorts the results; a View, Options tab which opens the Electronic Northern Options window for selecting viewing options; and a View, Plot Options tab which opens the Plot Option window where display options for the plot can be selected.

In another preferred embodiment of the present invention, the application further comprises an Expression Data Tool, which allows the user to retrieve and display expression data values (individual or aggregate) for one or more sample sets and one or more gene sets. The expression values preferably can be displayed in a table or overlaying a pathway or chromosome map.

The Expression Data Tool identifies gene expression data for genes and sample sets of interest, and extracts the individual (raw), mean, or median expression values for them (including the quantitative expression intensity and present/absent calls). The resulting data can either be displayed within the application of the present invention or exported to be used with analyses outside of the application.

The results for the selected samples are preferably displayed in the Expression Data tab, which preferably presents a statement of the number of rows in the results, a statement about the type of normalization used, and a table of result genes.

Preferably the Expression Data Tool provides a Show Details Panel which, if selected, displays detailed information about selected gene fragment, including Affy Fragment, which includes Attributes and Known Gene data; Sample Details, which include Attributes, Experiments, Sample, and Donor data; Sequence Cluster; and Plot.

The data content of the Expression Data can preferably be further refined by selecting additional options, including Aggregate Values (Sample Set) and Individual Sample(s).

The application of the present invention also preferably includes the ability to view pathways with regard to Expression Data Tool. The Pathway Viewer tab presents a pathway display where expression values are overlaid on known pathways. The content that the Pathway Viewer tab displays can be further refined by selecting viewing options, including Raw Expression Values (Selected Affy Fragments Only) which, if selected, the raw expression levels will be displayed for each Affymetrix fragment in the selected gene set that overlaps the pathway, over all samples in the input sample set(s), and Raw Expression Values (All Affy Fragments in Pathway) which, if selected, the raw expression levels will be displayed for all Affymetrix fragments that map to the pathway, regardless of the gene set selected, over all samples in the input sample set(s).

The application of the present invention also preferably includes the ability to view chromosome maps with regard to Expression Data Tool. The Chromosome Viewer tab presents a display that renders expression values over a chromosome map. The content that the Chromosome Viewer tab displays can be further refined by selecting viewing options, including Raw Expression Values for Samples which, if selected, raw expression values for all the samples will be displayed, and Call Values for Samples which, if selected, call values for all the samples will be displayed.

A gene set or selected genes can preferably be saved to use with other analyses.

Preferably there are a variety of menu options that are available for use with the Expression Data Tool, including a File, New tab which opens a new Expression Data Tool window; a File, Save Gene Sets tab which opens the Save Gene Set As window in which a gene set of the results can be saved; a File, Save Selected Genes tab which opens the Save Gene Set As window in which selected gene fragments can be saved as a unique gene set; a File, Export tab which provides options for exporting the results; a File, Invoke tab which provides options for accessing third-party applications in which to view the results; a File, Print tab which opens the Page Setup window for setting up the page layout and printing the results; and a File, Close tab which closes the Expression Data Tool window.

Preferably the Expression Data Tool menu further includes a View, Parameters tab which accesses the Parameters tab; a View, Expression Data tab which accesses the Expression Data tab; a View, Pathway Viewer tab which accesses the Pathway Viewer tab; a View, Chromosome Viewer tab which accesses the Chromosome Viewer tab; a Show Details Panel tab which, if selected, displays the details in the Expression Data panel; a View, Select Display Attributes tab which opens the Select Display Attributes window where columns to display in the results can be selected; a View, Gene Set Mask Add/Remove Mask tab which opens the Add/Remove Gene Set Mask window in which to add or remove a gene set mask to the results; a View, Remove Selected Genes tab which removes the selected genes from the currently selected results; a View, Remove Unselected Genes tab which removes the unselected genes from the results; a View, Reset to Original Gene Set(s) tab which resets the results to their original state; a View, Sort By tab which sorts the results; a View, Options tab which opens the Expression Data Tool Options window for selecting viewing options; and a Plot Options tab which opens the Plot Option window where display options for the plot can be selected.

In another preferred embodiment of the present invention, the application further provides the ability to perform a Contrast Analysis, which is a “pattern matching” tool used to find genes that fit a pattern of expression across sample sets.

Contrast analysis generalizes the significance testing performed in the fold change analysis tool to test for patterns of expression involving two or more sample sets. The specific statistical method is an ANOVA model with expression values used as response variable and sample sets used to define group effects. Contrasts are used to specify patterns among group effects. If the sample sets are labeled A, B, and C, for example, the contrast weight vector {1, −2, 1 } specifies a null hypothesis of the form:

H(0): 1×mean SΣA(log Es)−2×mean SΣB(log ES)+1×mean SΣC(log ES)0

where ES is the expression level of the gene being tested for samples.

(As is the case with the fold change analysis, the test is performed on the logarithm of the expression values, not on the expression values directly. This is done to increase the statistical power of the method. Negative expression values are mapped to negative log values by taking the log of the absolute value and multiplying by −1. Expression values whose absolute values are less than 1 are replaced by 0.)

The null hypothesis is used to calculate a t-statistic for each pattern in a method similar to the familiar two-sample t-test. The value of the t-statistic increases according to the adherence to the pattern of the expression values of the gene over the samples in the sample sets. Large positive t-scores mean that the pattern of variation of expression values between sample sets, relative to the amount of variation within sample sets, closely follows the pattern represented by the contrast. Large negative t-scores mean that the pattern of variation is the inverse of the pattern represented by the contrast. This would happen, for instance, for the contrast {−1, 1 } (representing an increase of expression in Sample Set 2 relative to Sample Set 1), for genes whose expression was decreased in Sample Set 2. Finally, t-scores close to zero mean that the gene's expression pattern matches neither the contrast pattern nor its inverse, or that the amount of variation between sample sets is comparable to or smaller than the variation within sample sets.

Multiple contrasts can be tested in parallel, in order to rank genes according to how well they fit any of several patterns. The user has the option of ranking the genes by either the maximum t-score (corresponding to selecting genes by the best fit to a single pattern) or the minimum t-score (corresponding to selecting genes by their ability to fit all of the patterns).

The contrasts can be specified either by using a graphical tool or by directly entering the contrast weights expert users familiar with the method). Due to the mathematical constraints of the model, some patterns specified by the graphical tool may lead to unexpected results.

As described below, in these cases a warning will be issued at the time the pattern is specified, and the user is encouraged to examine the output of the analysis carefully to ascertain that the result generated corresponds to what he/she is looking for.

If requested, a p-value is estimated by a randomization trial over sample assignments to sample sets to assess the significance of the maximum t-score over all the genes and patterns requested.

The “Leave One Out Plot” is a tool for detecting outlier samples. It allows the user to identify samples that behave so differently from the other members of their sample sets that they have a disproportionate effect on the results of the contrast analysis. These samples can be analyzed further with other tools to determine if there are problems with the sample data quality.

The contrast analysis is a generalization of the fold change analysis, and operates on multiple groups of sample sets, performing a similar series of fits for each group and comparing their levels using a set of contrasts specified by the user. Once these group effects are calculated, the results are multiplied by the contrasts, and a new statistic is calculated, which is similar in form and meaning to the two-sample t statistic.

Contrast Analysis can be seen as an extension of fold change analysis. The fold change tool used to compare expression levels between two experimental conditions, or groups. This tool computes t-scores (not exposed to the user) that can be used to rank the strength of the difference between conditions for an individual gene. These t-scores are the basis of a t test comparing the difference of the group means against the null hypothesis that the means of the populations sampled by the experiments are equal, taking into account the group variance, and are the input into the algorithm that determines the p-values reported.

Since the logarithms of the data points are taken before the analysis is performed, the fold change is determined based on the ratio of the geometric means of the data in the two groups compared. For two groups {A} and {B}, the t-score is simply the difference between the mean of {log A} minus the mean of {log B}, divided by the root mean square of the variations of the two logged groups, weighted by the number of points in each group. Take:

M(A)=mean {logA}

V(A)=variance {log A}=standard deviation {log A} squared

N(A) number of points in A

and define similar values for group B. The null hypothesis is given for this test as:

H(0):M(A)−M(B)=0

The t-score is given by

t(A,B)=[M(A)−M(B)]/sqrt[V(A)/N(A)+V(B)/N(B)]

The fold change reported is exp(M(A)−M(B)).

To summarize the t-score calculation, the larger the difference of the log means relative to the log variances, the larger the absolute value of the t-score, and the more likely that the groups actually are different. The null hypothesis for the t test is that M(A)=M(B), or, equivalently, that t(A,B)=0. The higher the t-score, the lower the p-value. The p-value reported by the fold change tool is based on assuming that the 2 groups {log A} and {log B} are normally distributed, and the weighting factor takes into account a possible difference in the group sizes. Summarizing an experimental group's characteristics with its estimated mean and variance is a powerful technique for reducing the complexity of analyzing such comparisons.

This idea can be extended to more than two conditions (or groups, or sample sets) using the statistical method of contrast analysis, which uses the results of a one-way analysis of variance (ANOVA) on the individual groups. Whereas the simple t test compares two group means, a contrast analysis compares the relative levels of a large number of group means to a model specified by the user. Many situations that arise in the analysis of expression data are amenable to such analysis, if the method is understood properly. There are limitations to this method, and care must be taken to understand them to ensure that the results are interpretable. This method is particularly useful when comparing the fits of two or more models to the data. As in the case with the two group t test, a ranking score (called a t-score, or t-like statistic) is generated that allows a comparison of how well a pattern matches the data. These patterns are parameterized by a contrast (a series of coefficients for the group means).

Since the test relies on the null hypothesis that all group means are the same (that is, there is no difference in expression among the groups), the only valid contrasts are those in which the means are weighted with coefficients that sum to zero. Ranking the genes for the comparisons in decreasing order of t-score should give the same order as ranking the genes in increasing order of p-value.

The contrast analysis tool uses a more sophisticated algorithm to calculate p-values, one not based on the assumption that the measurements are normally distributed within groups. Instead, the p-values are calculated by computing a distribution of the maximum t-score over all genes and all patterns. First, the expression values for the different genes are randomly reassigned many times, and the entire set of t-scores is recomputed. The maximum is found for each iteration, and this distribution of t-values is used to estimate the p-value for the maximum t-score reported. The number of mathematically independent contrasts that can be tested against is simply the number of groups (G) minus 1. In the case of the simple t test, G=2 and only one contrast exists. As G increases, so does the number of independent contrasts.

However, any contrast that is a linear combination of these independent contrasts is valid within the theory. Included within the sets of valid contrasts are those which include coefficients equaling 0. These cases require special attention, since a weighting of 0 removes that value from the contrast calculation in the numerator of the t-score, while including that group's variance in the denominator.

Contrast Analysis Algorithm

1. Perform a logarithmic transformation of the data points Eraw(n,g), the raw expression values for gene g in sample n. The transformed values are given by:

E(n,g)=log(Eraw(n,g)) for Eraw(n,g)>1=0 for |Eraw(n,g)<=1 =−log(−Eraw(n,g)) for Eraw(n,g)<1

2. Generate the X matrix of group assignments. This consists of N rows by K columns, where N is the total number of individual samples, and K is the total number of groups. In the kth column, the nth row contains 1 if the nth sample is in group k, and 0 if not.

3. This matrix is the basis of a family of models (one for each gene g):

E(g)=Xm(g)+ε(g)

where E(g) is a (N×1) row vector of transformed expression observations for gene g, m(g) is a (1×K) column vector of the group means for gene g, and ε(g) is the residual error, assumed to be normally distributed about 0 with variance σ²(g). If a value is missing in the row vector E(g) (indicated by a “N” or “U” call in the presence call matrix), the calculation will remove it from the matrix and proceed as though it were not in the original list.

4. These models are used to generate the group means estimates e(m(g)). These are solutions to the least squares normal equations:

X′Xe(m(g))=X′E(g)

Here X′is the transpose of X. Note that the numerical method of solution for this equation is not specified here; there are many methods of solving this equation. The current implementation of the algorithm uses QR decomposition.

5. An estimate of the variance from the fit is obtained by calculating the mean residual sum of squares:

e(σ²(g))(E(g)−e(m(g))X)(E(g)−e(m(g))X)′/(N−(g)−K)

6. The comparative t-scores are calculated by using the contrast matrix C, a(K×C) matrix of the C desired contrasts. For each contrast, the cth column consists of a coefficient for the kth group in the kth row. The numerators of the c t-scores are given by the rows of the (1×C) vector N(g):

N(g)=Ce(m(g))

The denominators are given by the square root of the rows of the (1×C) vector V(g):

V(g)=|eσ²(g)) diag(CInverse(X′X)C′|.

Here diag(X) extracts the diagonal elements of a matrix X. It generates a vector of t's whose cth component is given by:

T(g,c)=N(g,c)/sqrt(V(g,c)).

Note that unlike the case of the fold change t-scores, the assumption made here is of equal variances across groups.

7. If C>1, the maximum or minimum t-score is selected out of the tc's for each gene, depending on the user input for which comparison is desired. The contrast index c is noted for the contrast that satisfies the minimum or maximum criterion.

8. These maximum or minimum t-scores are then combined across all genes to generate a list Tmax(g) of length G indicating which patterns are most/least strongly matched.

9. If the user has requested p-values, these are generated by a procedure whereby the individual measurements are assigned with replacement to different samples for 1000 trials. For each randomization trial j, calculate the maximum t-score for each g: Tmax(g, j). Take the maximum of all these to generate a top ranking t-score Tmax(j). These are pooled together across all the randomization trials and genes to generate a distribution of maximal t-scores Tmaxpooled. The original t-scores generated in Step 8 are compared to their rank in this pooled distribution. Divide the number of points in the pooled distribution with a greater T-value by the total number of points in the pooled distribution to estimate the p-value, that is:

p(g)=(number Tmaxpooled>t)/G*1000

The Leave One Out Plot consists of repeating the contrast computation N times. For each of these N cases, one of the N samples is left out of the calculation and a ranked list

r(g)=rank of g in Tmax(g)

of maximum t-scores is generated. If each gene g has a rank r(g,0) with no samples left out and rank r(g,n) with sample n left out, then compute for each gene the value:

d(g,n)=|r(g,n)−r(g,0)|

One calculates the median value of d over all genes:

d(n)=median(d(g,n))

This value is used as a summary statistic to estimate the effect that leaving one sample out has on the results of the analysis (namely, the ranking of the genes according to the contrasts specified).

In performing a Contrast Analysis, one first selects sample and gene sets for the analysis. Then, one defines the contrast pattern(s). A preferred method for accomplishing this is to select either highest or lowest for the “T-score among contrasts.” Using the maximum T-score to rank genes (that is, highest) functions as a logical OR pattern search; that is, genes are ranked high if a large T-score is obtained for any of the input patterns. Alternatively, genes can be ranked by the minimum T-score. This functions as a logical AND on the input patterns, and is useful when the user wants to select for a set of genes that match one or more patterns equally well.

Preferably there are two ways of defining the contrast patterns: specifying a graphical pattern and entering contrast weights. Specifying a graphical pattern option presents a graphical representation of the contrast pattern which makes it easier to visualize the contrast pattern(s) being used for the analysis. Preferably, the relative direction of the pattern is low, high, or neutral for each of the selected sample sets. The pattern represents the change in mean expression value over each checked sample set. Only the relative vertical order of the values is significant in the pattern. The pattern is converted to a “contrast,” which is a list of integer weights, one for each input sample set.

The contrast weights are positive or negative numbers, one for each input sample set, whose values follow the same relative order as the heights of the boxes. The values are scaled and adjusted so that the sum of the weights is zero. Zero weights are assigned for sample sets that are not used in the pattern. All of the sample sets displayed of the contrast analysis window will be included in the analysis. For each sample set a mean and residual will be calculated. The residuals from all sample sets will be pooled for use in the t-score calculation, regardless of the pattern and whether or not the sample set was selected. This includes samples whose contrast weight is 0. Only the rank order of mean log expression levels between the sample sets is considered when converting the pattern to a contrast. For example, the following two patterns are considered equivalent; they correspond to the same vector of contrast weights, {−1, 2, −1 }. Simply put, both patterns will select for genes whose mean log expression over Sample Sets 1 and 3 is the same, and is lower than the mean log expression for Sample Set 2.

The correspondence between patterns and contrast vectors is not always so intuitive. A confusing example is the pattern which pattern corresponds to the contrast weight vector {−1, 0, 1 }. It will select for genes whose mean log expression level in Sample Set 1 is lower than that in Sample Set 3. The zero weight for Sample Set 2 means that the mean log expression value over this set is not taken into account. The t-score which results will be independent of the mean log value for the second sample set, contrary to the appearance of the pattern. For this reason, a warning is preferably issued:

In the entering contrast weights option, an advanced interface is provided to allow for entering the weights directly. One enters one contrast weight for each sample set. Normalization can also be used in the analysis, and the p-value can also be computed.

When the contrast analysis computation is complete, the results will be displayed in the Results tab. The Result tab displays the results of the contrast analysis. Preferably the genes from the input gene set(s) are sorted in decreasing order of either the maximum or minimum t-score, as specified. in Step 2 of the analysis. This view presents the following information: a table of result genes, including: the total number of rows displayed in the results, the gene attributes selected by the user, a t-score column for each contrast pattern, the maximum and minimum t-score from the t-score columns, an index of the maximum t-score.

Preferably, the contrast analysis aspect of the application of the present o9nvention also provides a Leave One Out Plot. The Leave One Out Plot is a tool for detecting outlier samples. It allows the user to identify samples that behave so differently from the other members of their sample sets that they have a disproportionate effect on the results of the contrast analysis. These samples can be analyzed further with other tools to determine if there are problems with the sample data quality or if these samples are unique in some way.

Samples that behave very differently from the other members of their sample sets will be associated with bars that are taller than most of the other bars in the plot. These samples can be selected and “removed.” This causes the tool to recompute all the T-scores and ranks based on modified input sample sets, from which the selected samples have been removed, without actually changing the underlying sample sets in the workspace.

In performing the analysis, the application iterates over the samples in the input sample sets. For each sample, the application removes the sample from its sample set, recomputes the t-scores for all contrasts for the N genes, re-ranks the genes by maximum or minimum t-score, subtracts each gene's original ranking from its new rank, and computes the absolute value of the difference. The median of these absolute rank differences for the N genes is then computed. Finally the median is reported for each sample in the Leave One Out plot.

Preferably there are a variety of menu options that are available for use with the Contrast Analysis, including: a File, New tab which opens a new Contrast Analysis window; a File, Open tab which opens the Select Contrast Analysis window from which a previously saved contrast analysis can be opened; a File, Save Contrast Analysis tab which opens the Save Contrast Analysis As window where the contrast can be named and saved; a File, Save Gene Set tab which opens the Save Gene Set As window in which the resulting gene set from the Contrast Analysis can be saved; a File, Save Selected Genes tab which opens the Save Gene Set As window in which selected gene fragments can be saved as a unique gene set; a File, Export tab which provides options for exporting the results; a File, Invoke tab which provides options for accessing third-party applications in which to view the results; a File, Print tab which opens the Page Setup window for setting up the page layout and printing the results; and a File, Close tab which closes the Contrast Analysis window.

Preferably the Contrast Analysis menu further includes a View, Compute Form tab which opens the Compute tab; a View, Results tab which opens the Results tab; a View, Show Details Panel tab which toggles to display the details panel in the Results tab; a View, Select Display Attributes tab which opens the Select Display Attributes window where columns to display gene attributes and data values can be selected; a View, Gene Set Mask Add/Remove Mask tab which opens the Add/Remove Gene Set Mask window in which a masking gene set can be applied to or removed from the input gene set; a View, Remove Selected Genes tab which removes the selected genes from the currently displayed results; a View, Remove Unselected Genes tab which removes the unselected genes from the results; a View, Reset to Original Gene Set(s) tab which resets the results to their original state; a View, Sort By tab which sorts the results; and a Plot Options tab which opens the Plot Option window.

Additional preferred aspects of the present invention is the fragment index and the gene query attribute tree. Aspects of these components of the present invention include cross-species homology in the gene index; co-clustered sequences and searching by GenBank Accession; BLAST Hits and Warnings; gene ontologies; and gene query attribute tree.

Cross-species homology is represented in two principal ways in the gene index: a relationship between Known Genes that uses curated lists of homologous genes from the Mouse Genome Database (MGD) and a relationship between Sequence Clusters that uses shared similarity to protein sequences.

The lists from MGD are of homologous pairs of mouse and human genes, and of mouse and rat genes. In the Gene Index, “human−>rat” homologies are also included by transitive extension of the “rat−>mouse” and “mouse−>human” relationships. Gene fragments (i.e., probe sets) corresponding to cross-species homologies are accessible through the Cross Sp. Homologous Fragments query option, which is under Homologies. There can be extended to other species by exporting the data and then re-importing the list as a gene set in the context of the other species.

These gene-level homologies are accessible both for query and display through the Known Gene query option, and are also displayed in the Attributes details panel for a given individual fragment.

If two sequence clusters share homology to the same protein sequence, as determined by the PROTSIM data from UniGene, each points to the other as a Homologous Cluster. Homologous clusters may be of the same species or of different species.

Frequently, users of the gene index have a GenBank accession of a sequence, and would like to find fragments (probe sets) on the chips that correspond to this sequence. An appropriate way to do this is by searching co-clustered sequences, under AFFX Gene Fragment. For a given Affymetrix gene fragment, Co-clustered Sequences contain all sequences in UniGene which are in the same sequence cluster (or clusters) as the fragment. This provides very good coverage of ESTs. If an exact accession is known (or a list of accessions is available using the Import by Attribute method, using “matches” is considerably faster.

Many Affymetrix Gene Fragments may correspond to the same Sequence Cluster. To find Affymetrix Gene Fragments that are in the same Sequence Cluster as a given fragment, search using Co-clustered AFFX fragments (under Related Other AFFX Fragments).

Co-clustered AFFX fragments may include fragments in other chipsets in addition to the chipset one is starting with. For example, the co-clustered fragments of a given Affymetrix Gene Fragment in the Hu42K chip set may include fragments in both the Hu42K chip set and the HG_U95 chip set.

The data in BLAST Hits and Warnings comes from two sources. One is a list of problematic fragments provided by Affymetrix. The other is a BLAST of the sif sequence (“Tiled Region Sequence” in the fragment detail view) against NCBI's Refseq database of full-length transcripts. The oligomer probes on the chip are derived from a subset of the sif sequences. BLAST hits which are above a sensitivity threshold (97% identity over greater than 80% of the sif sequence length) fall into three categories: if the match of the sif sequence is to the antisense strand, the Warning Message is set to “Matches wrong strand;” if the match is to the sense strand, the minimum, maximum, and mean distances of the match to the 3-prime end of the transcript are calculated and entered in the Min. Distance, Mean Distance, and Max. Distance fields; if the mean distance to the 3-prime end is greater than 1000 nucleotides, the Warning Message is set to “Probes far from 3prime end.”

In all cases, the GenBank accession of the Refseq sequence is entered in the Ref Seq ID field, and the symbol of the corresponding gene appears in the Gene field. The Fragment Warning attribute of a Affymetrix Gene Fragment is derived from the data in BLAST Hits and Warnings. The default value of Fragment Warning is “No.” It is set to “Yes” if: the fragment is on Affymetrix' list of problematic fragments OR there are BLAST hits with warnings but none without warnings

The Gene Ontology Consortium (http://genome-www.stanford.edu/GO/) is a public project dedicated to providing a dynamic controlled vocabulary that can be applied to all eukaryotes even as knowledge of gene and protein roles in cells is accumulating and changing. An ontology of biological terminology provides a model of biological concepts that can be used to form a semantic framework for many data storage, retrieval, and analysis tasks. Such a semantic framework could be used to facilitate seamless integration of various heterogeneous bioinformatics data, and allows uniform querying across them.

Gene Ontology (GO) terms are defined by three different principles: molecular function: describes the tasks performed by individual gene products; examples are transcription factor and DNA helicase; biological process: describes broad biological goals and the process is accomplished by ordered assemblies of molecular functions; example is purine metabolism process; and molecular component: encompasses sub-cellular structures, locations, and macromolecular complexes; examples include nucleus, telomere, and origin recognition complex.

Various preferred embodiments of the invention have been described in fulfillment of the various objects of the invention. It should be recognized that these embodiments are merely illustrative of the principles of the invention. Numerous modifications and adaptations thereof will be readily apparent to those skilled in the art without departing from the spirit and scope of the present invention.

What is claimed is:

1. A method of analyzing gene expression, gene annotation, and sample information in a relational format supporting efficient exploration and analysis, the method comprising:
providing a data warehouse which comprises a gene expression database for storing quantitative gene expression measurements for tissues and cell lines screened using various assays; a clinical database for storing information on bio-samples and donors; and a fragment index for biological properties for DNA fragments;
providing a connector which permits loading of more than one source of gene expression, gene annotation, and sample information, receiving a query regarding gene expression of one or more DNA fragments;
determining the level of gene expression of the one or more DNA fragments;
correlating the level of gene expression with the clinical database and the fragment index; and
displaying the results of said correlation.

2. The method of claim 1, wherein the connector permits registering of more than one source of gene expression, gene annotation, and sample information and extraction of a list of experiments from the more than one source of gene expression, gene annotation, and sample information.

3. The method of claim 2, wherein the connector permits the refreshing of the list of experiments from the more than one source of gene expression, gene annotation, and sample information.

4. The method of claim 3, wherein the connector permits extraction and checking of a list of selected experiments from the more than one source of gene expression, gene annotation, and sample information.

5. The method of claim 4, wherein the method further comprises providing a staging database and wherein the connector permits migration of the more than one source of gene expression, gene annotation, and sample information from the staging database into the data warehouse.

6. The method of claim 5, wherein the connector permits loading of the more than one source of gene expression, gene annotation, and sample information into an analysis engine.

7. The method of claim 6, wherein the connector permits loading of the more than one source of gene expression, gene annotation, and sample information from XML files.

8. The method of claim 7, wherein the connector permits checking for consistency the more than one source of gene expression, gene annotation, and sample information.

9. The method of claim 8, wherein the method further comprises
providing a sample data editor and
wherein the connector permits updating of sample data with the sample data editor.

10. The method of claim 9, wherein the connector permits the providing of association between experiments and samples.

11. The method of claim 10, wherein the connector permits acquisition of such association from an XML sample file.

12. The method of claim 10, wherein the method further comprises
providing an expression data migration tool and
wherein the connector permits acquisition of such association from the an expression data migration tool.

13. A computer system comprising
a data warehouse which comprises a gene expression database for storing quantitative gene expression measurements for tissues and cell lines screened using various assays; a clinical database for storing information on bio-samples and donors; and a fragment index for biological properties for DNA fragments;
a user interface capable of receiving a query regarding gene expression of one or more DNA fragments and displaying the results of a correlation of the level of gene expression with the clinical database and the fragment index; and
a connector which permits loading of more than one source of gene expression, gene annotation, and sample information.

14. The computer system of claim 13, wherein the connector permits registering of more than one source of gene expression, gene annotation, and sample information and extraction of a list of experiments from the more than one source of gene expression, gene annotation, and sample information.

15. The computer system of claim 14, wherein the connector permits the refreshing of the list of experiments from the more than one source of gene expression, gene annotation, and sample information.

16. The computer system of claim 15, wherein the connector permits extraction and checking of a list of selected experiments from the more than one source of gene expression, gene annotation, and sample information.

17. The computer system of claim 16, wherein the computer system further comprises
a staging database and
wherein the connector permits migration of the more than one source of gene expression, gene annotation, and sample information from the staging database into the data warehouse.

18. The computer system of claim 17, wherein the connector permits loading of the more than one source of gene expression, gene annotation, and sample information into an analysis engine.

19. The computer system of claim 18, wherein the connector permits loading of the more than one source of gene expression, gene annotation, and sample information from XML files.

20. The computer system of claim 19, wherein the connector permits checking for consistency the more than one source of gene expression, gene annotation, and sample information.

21. The computer system of claim 20, wherein the computer system further comprises
a sample data editor and
wherein the connector permits updating of sample data with the sample data editor.

22. The computer system of claim 21, wherein the connector permits the providing of association between experiments and samples.

23. The computer system of claim 22, wherein the connector permits acquisition of such association from an XML sample file.

24. The computer system of claim 22, wherein the computer system further comprises
an expression data migration tool and
wherein the connector permits acquisition of such association from the an expression data migration tool.
